SEQUENCE LISTING (15 sequences)

SEQ ID NO: 1 - protein, 23 aa, Artificial Sequence
  Exemplary Mcl-1 Binding Domain sequence
RPEIWMTQGLRRLGDEINAYYAR

SEQ ID NO: 2 - protein, 23 aa, Artificial Sequence
  Exemplary Mcl-1 Binding Domain sequence
RPEIWLTQSLQRLGDEINAYYAR

SEQ ID NO: 3 - protein, 23 aa, Artificial Sequence
  Exemplary Mcl-1 Binding Domain sequence
RPEIWLTQHLQRLGDEINAYYAR

SEQ ID NO: 4 - protein, 23 aa, Artificial Sequence
  Exemplary Mcl-1 Binding Domain sequence
RPEIWMGQGLRRLGDEINAYYAR

SEQ ID NO: 5 - protein, 23 aa, Artificial Sequence
  Exemplary Mcl-1 Binding Domain sequence
RPEIWLGQSLQRLGDEINAYYAR

SEQ ID NO: 6 - protein, 23 aa, Artificial Sequence
  Exemplary Mcl-1 Binding Domain sequence
RPEIWLGQHLQRLGDEINAYYAR

SEQ ID NO: 7 - protein, 23 aa, Artificial Sequence
  Exemplary Mcl-1 Binding Domain sequence
RPEIWITQELRRIGDEFNAYYAR

SEQ ID NO: 8 - protein, 23 aa, Artificial Sequence
  Exemplary Mcl-1 Binding Domain sequence
RPEIWMTQELRRIGDEFNAYYAR

SEQ ID NO: 9 - protein, 23 aa, Artificial Sequence
  Exemplary Mcl-1 Binding Domain sequence
RPEIWITQGLRRIGDEFNAYYAR

SEQ ID NO: 10 - protein, 23 aa, Artificial Sequence
  Exemplary Mcl-1 Binding Domain sequence
RPEIWITQELRRLGDEFNAYYAR

SEQ ID NO: 11 - protein, 23 aa, Artificial Sequence
  Exemplary Mcl-1 Binding Domain sequence
RPEIWITQELRRIGDEINAYYAR

SEQ ID NO: 12 - protein, 11 aa, Artificial Sequence
  TAT translocation domain
YGRKKRRQRRR

SEQ ID NO: 13 - protein, 16 aa, Artificial Sequence
  ANT translocation domain sequence
RQIKIWFQNRRMKWKK

SEQ ID NO: 14 - protein, 37 aa, Artificial Sequence
  profiling peptide sequence
YGRKKRRQRRRGGGRPEIWMTQGLRRLGDEINAYYAR

SEQ ID NO: 15 - protein, 37 aa, Artificial Sequence
  profiling peptide sequence
RPEIWMTQGLRRLGDEINAYYARGGGYGRKKRRQRRR

(19) United States
(12) Patent Application Publication
(10) Pub. No.: US 2020/0276174 A1
(43) Pub. Date: Sep. 3, 2020

(54) CYCLIN-DEPENDENT KINASE INHIBITORS IN COMBINATION WITH ANTHRACYCLINES FOR TREATMENT OF CANCER

(71) Applicant: TOLERO PHARMACEUTICALS, INC., Lehi, UT (US)

(72) Inventors: David J. BEARSS, Alpine, UT (US); Steven L. WARNER, Sandy, UT (US); Clifford J. WHATCOTT, West Jordan, UT (US)

(21) Appl. No.: 16/708,366

(22) Filed: Dec. 9, 2019

Related U.S. Application Data
(63) Continuation of application No. 16/382,726, filed on Apr. 12, 2019, now abandoned.
(60) Provisional application No. 62/657,545, filed on Apr. 13, 2018.
(60) Provisional application No. 62/688,228, filed on Jun. 21, 2018.

(51) Int. Cl. A61K 31/453; A61K 31/351; A61K 9/00; A61P 35/00; A61K 31/7068

(52) U.S. Cl. CPC A61K 31/453; A61K 31/351; A61K 31/7068; A61P 35/00; A61K 9/0019

(57) ABSTRACT

Methods for treating cancer by administration of two or more therapeutic agents are provided. The two or more therapeutic agents include a cyclin-dependent kinase inhibitor (e.g., alvocidib) and an anthracycline (e.g., daunorubicin or idarubicin). Kits comprising the two or more therapeutic agents that can be used to perform such methods are also provided.

### RELATED APPLICATIONS

This application is a continuation of U.S. Nonprovisional application Ser. No. 16/382,726, filed Apr. 12, 2019, which claims the benefit of U.S. Provisional Application No. 62/657,545, filed Apr. 13, 2018, and U.S. Provisional Application No. 62/688,228, filed Jun. 21, 2018. The entire disclosures of the above applications are incorporated herein by reference.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910208_437C1_ST25.txt. The text file is 5.39 KB, was created on Dec. 9, 2019, and is being submitted electronically via EFS-Web.

### BACKGROUND

Cyclin-dependent kinases, or CDKs, are a family of proteins that form complexes involved in either cell cycle progression or transcription regulation. CDK9 activates transcription elongation by phosphorylating the C-terminal domain of RNA polymerase II. Alvocidib is a potent inhibitor of CDK9. By inhibiting CDK9, alvocidib downregulates the transcription of target genes, including the myeloid cell leukemia-1 (MCL-1) gene. In MCL-1-dependent acute myeloid leukemia (AML), downregulation of MCL-1 protein expression can trigger apoptosis of leukemic blasts.

There is a need to translate the mechanistic activity of alvocidib into novel treatment regimens for hematologic cancers.

### SUMMARY

In brief, embodiments of the present disclosure provide methods for treatment of cancer comprising administering two or more different therapeutic agents. For example, embodiments of the present disclosure include methods of treating cancer in a subject by administering an effective amount of a cyclin-dependent kinase (CDK) inhibitor and an effective amount of an anthracycline. In some embodiments, the disclosure provides a method for treating a cancer in a subject in need thereof, the method comprising administering to the subject an effective amount of a cyclin-dependent kinase (CDK) inhibitor, an effective amount of an anthracycline, and an effective amount of a nucleoside analog. In certain embodiments, the CDK inhibitor is a CDK9 inhibitor. In particular embodiments, the CDK9 inhibitor is alvocidib, or a prodrug thereof, the anthracycline is daunorubicin, and the nucleoside analogue is cytarabine.

In some embodiments, the disclosure provides a method for treating a hematologic cancer in a subject in need thereof. The method comprises administering to the subject a treatment comprising an effective amount of each of: alvocidib, or a prodrug thereof, or a pharmaceutically acceptable salt of the foregoing; daunorubicin or idarubicin, or a pharmaceutically acceptable salt of the foregoing; and cytarabine, or a pharmaceutically acceptable salt thereof.

In a more specific embodiment, the disclosure provides a method for treating previously untreated AML in a subject in need thereof. The method comprises administering to the subject a treatment comprising:

from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;

from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the treatment; and

from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment. The treatment results in the subject being measurable residual disease (MRD)-negative.

In another specific embodiment, the disclosure provides a method for treating intermediate-risk AML or high-risk AML in a subject in need thereof. The method comprises administering to the subject a treatment comprising:

from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;

from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the treatment; and

from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment.

Kits comprising an effective amount of a CDK inhibitor; an effective amount of an anthracycline; and written instructions for administration of the CDK inhibitor and the anthracycline are also provided.

In some embodiments, the present disclosure provides a kit comprising alvocidib, or a prodrug thereof, or a pharmaceutically acceptable salt of the foregoing; daunorubicin or idarubicin, or a pharmaceutically acceptable salt of the foregoing; cytarabine, or a pharmaceutically acceptable salt thereof; and written instructions for administering the alvocidib, or a prodrug thereof, or a pharmaceutically acceptable salt of the foregoing, daunorubicin or idarubicin, or a pharmaceutically acceptable salt of the foregoing, and cytarabine, or a pharmaceutically acceptable salt thereof, to a subject in need of treatment for a hematologic cancer.

In some embodiments, the present disclosure provides a combination therapy comprising alvocidib, or a prodrug thereof, or a pharmaceutically acceptable salt of the foregoing; daunorubicin or idarubicin, or a pharmaceutically acceptable salt of the foregoing; and cytarabine, or a pharmaceutically acceptable salt thereof.

These and other aspects of the disclosure will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 shows differing activities of alvocidib, cytarabine, and daunorubicin in various AML cell lines.

FIG. 2 shows data from an in vitro model of MCL-1 expression during administration of an alvocidib+cytarabine+daunorubicin regimen.

FIG. 3 shows apoptosis/caspase activity in cells treated with various combinations of drugs, including cytarabine and daunorubicin.

FIG. 4 shows results of in vivo testing of alvocidib, cytarabine, and daunorubicin in the MV4-11 AML xenograft model.

FIG. 5 shows a Phase Ib clinical trial design in patients with newly diagnosed AML.

### DETAILED DESCRIPTION

The present disclosure relates to methods of treating cancer by administering a combination of a cyclin-dependent kinase (CDK) inhibitor, such as alvocidib, or a prodrug thereof, and an anthracycline, such as daunorubicin. In embodiments, the cancer treatment further comprises administering a nucleoside analog, such as cytarabine.

In the following description, certain specific details are set forth in order to provide a thorough understanding of various embodiments of the disclosure. However, one skilled in the art will understand that the disclosure may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

“Subject” includes humans, domestic animals, such as laboratory animals (e.g., dogs, monkeys, rats, mice, etc.), household pets (e.g., cats, dogs, rabbits, etc.), and livestock (e.g., pigs, cattle, sheep, goats, horses, etc.), and non-domestic animals (e.g., bears, elephants, porcupines, etc.). In embodiments, a subject is a mammal. In embodiments, a subject is a human.

A “cancer,” including a “tumor,” refers to an uncontrolled growth of cells and/or abnormal increased cell survival and/or inhibition of apoptosis which interferes with the normal functioning of the bodily organs and systems. “Cancer” (e.g., a tumor) includes solid and non-solid cancers. A subject that has a cancer or a tumor has an objectively measurable number of cancer cells present in the subject's body. “Cancers” include benign and malignant cancers (e.g., benign and malignant tumors, respectively), as well as dormant tumors or micrometastases.

“Metastasis” refers to the spread of cancer from its primary site to other places in the body. “Metastases” are cancers which migrate from their original location and seed vital organs, which can eventually lead to the death of the subject through the functional deterioration of the affected organs. Metastasis is a sequential process, where cancer cells can break away from a primary tumor, penetrate into lymphatic and blood vessels, circulate through the bloodstream, and grow in a distant focus (metastasize) in normal tissues elsewhere in the body. At the new site, the cells establish a blood supply and can grow to form a life-threatening mass. Metastasis can be local or distant. Both stimulatory and inhibitory molecular pathways within the tumor cell regulate this behavior, and interactions between the tumor cell and host cells in the new site are also significant.

A “pharmaceutical composition” refers to a formulation of one or more therapeutic agents and a medium generally accepted in the art for the delivery of the biologically active agent to subjects, e.g., humans. Such a medium may include any pharmaceutically acceptable carrier, diluent, or excipient. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

A “chemotherapeutic agent” or “anti-cancer agent” is a chemical which destroys cancer cells, or stops or slows the growth of cancer cells.

An “anthracycline” refers to a compound comprising the following core structure, which is optionally substituted at all available positions:

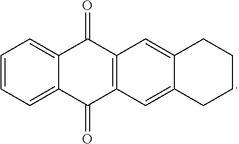 [SMILES: O=C1c2ccccc2C(=O)c2cc3c(cc21)CCCC3]

Exemplary anthracyclines include, but are not limited to, daunorubicin and idarubicin, or a pharmaceutically acceptable salt of the foregoing. In some embodiments, the anthracycline is daunorubicin, or a pharmaceutically acceptable salt thereof (e.g., daunorubicin hydrochloride). In some embodiments, the anthracycline is idarubicin, or a pharmaceutically acceptable salt thereof (e.g., idarubicin hydrochloride).

“Treating” or “treatment,” as used herein, refers to the administration of a medication or medical care to a subject, such as a human, having a disease or condition of interest, e.g., a cancer, and includes: (i) preventing the disease or condition from occurring in a subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, (e.g., pain, weight loss, cough, fatigue, weakness, etc.). As used herein, the terms “disease” and “condition” may be used interchangeably or may be different in that the particular malady or condition may not have a known causative agent (so that etiology has not yet been confirmed) and it is therefore not yet recognized as a disease but only as an undesirable condition or syndrome, wherein a more or less specific set of symptoms have been identified by clinicians.

“Effective amount” refers to the amount of a therapeutic agent which, when administered to a subject, such as a human, is sufficient to effect treatment. The amount of a therapeutic agent that constitutes an “effective amount” will vary depending on the therapeutic agent, the condition being treated and its severity, the manner of administration, the duration of treatment, or the age of the subject to be treated, but can be determined routinely by one of ordinary skill in the art based on his own knowledge and this disclosure. In embodiments, an “effective amount” effects treatment (e.g., treats, promotes, improves, increases, reduces, suppresses, and the like) as measured by a statistically significant change in one or more indications, symptoms, signs, diagnostic tests, vital signs, and the like. In other embodiments, an “effective amount” manages or prevents a condition as measured by a lack of a statistically significant change in one or more indications, symptoms, signs, diagnostic tests, vital signs, and the like.

As used herein, “statistically significant” refers to a p value of 0.050 or less when calculated using the Students t-test, and indicates that it is unlikely that a particular event or result being measured has arisen by chance.

The term “co-administration,” “administered in combination with,” and their grammatical equivalents, as used herein, encompass administration of two or more agents to a subject, such as an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts. Even if not specifically described in each instance, unless otherwise indicated (e.g., by the context), use of a therapeutic agent described herein optionally comprises use of a pharmaceutically acceptable salt of the therapeutic agent instead of, or in addition to, the parent compound.

“Pharmaceutically acceptable acid addition salt” refers to those salts which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid (e.g., L-(+)-tartaric acid), thiocyanic acid, p-toluenesulfonic acid, trifluoroacetic acid, undecylenic acid, and the like.

“Pharmaceutically acceptable base addition salt” refers to those salts which are prepared from addition of an inorganic base or an organic base to a free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“Prodrug” is meant to indicate a compound that may be converted under physiological conditions or by solvolysis to a biologically active compound. Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

The term “in vivo” refers to an event that takes place in a subject's body.

The term “in vitro” refers to an event that is performed or that takes place outside of a living organism (e.g., in a test tube, culture dish, etc.).

The use of the words “optional” or “optionally” means that the subsequently described event or circumstances may or may not occur, and that the description includes instances where the event or circumstance occurs and instances in which it does not.

### I. Methods

In certain embodiments, methods of the disclosure are useful for treating cancer in a subject. Therapeutic agents of the disclosure, when administered together or sequentially, treat a cancer in a subject (e.g., (i) prevent a cancer from occurring in the subject; (ii) arrest the cancer's development; (iii) cause regression of the cancer; or (iv) relieve the symptoms resulting from the cancer).

In embodiments, the methods of treatment disclosed herein comprise administering an effective amount of a CDK inhibitor and an effective amount of an anthracycline to the subject, thereby treating the subject's cancer.

In embodiments, the CDK inhibitor is a CDK4, CDK6, CDK7, CDK8, CDK9, CDK10, and/or CDK11 inhibitor. In some embodiments, the CDK inhibitor is a CDK7, CDK9 inhibitor, or both. In some embodiments, the treatment agent is dinaciclib (ACS Med. Chem. Lett. 2010 May 17; 1(5):204-8; Mol. Cancer Ther. 2010 August; 9(8):2344-53; Merck, Sharp and Dohme), AT7519 (J. Med. Chem. 2008 Aug. 28; 51(16):4986-99; Astex Pharmaceutical), palbociclib (J. Med. Chem. 2005 Apr. 7; 48(7):2388-406; Pfizer) or alvocidib, or a prodrug thereof (Int. J. Oncol. 1996 Dec. 9(6):1143-68). In embodiments, the CDK inhibitor is a CDK9 inhibitor. In some embodiments, the CDK9 inhibitor is a siRNA, alvocidib, or a prodrug thereof, dinaciclib, or a combination thereof. In some embodiments, the CDK9 inhibitor is a siRNA or alvocidib, or a prodrug thereof. In some embodiments the CDK9 inhibitor is alvocidib, or a prodrug thereof. In some embodiments, the CDK9 inhibitor is alvocidib, or a prodrug thereof, or a pharmaceutically acceptable salt of the foregoing. In some embodiments, the CDK9 inhibitor is alvocidib, or a pharmaceutically acceptable salt thereof (e.g., alvocidib hydrochloride).

In some embodiments, the anthracycline is daunorubicin, idarubicin, or a combination thereof. In certain embodiments, the anthracycline is daunorubicin. In certain embodiments, the anthracycline is idarubicin.

In some embodiments, the CDK inhibitor and the anthracycline are administered sequentially. In certain of these embodiments, the anthracycline is administered after the CDK inhibitor is administered. The treatment methods may optionally include a treatment break between administering the CDK inhibitor and administering the anthracycline.

In some embodiments, the methods further comprise administering to the subject an effective amount of a nucleoside analog. For example, in some embodiments, the nucleoside analog is a pyrimidine analog, such as cytarabine.

In some of the foregoing embodiments, the anthracycline and the nucleoside analog are administered sequentially. For example, in some aspects, the CDK inhibitor, the anthracycline, and the nucleoside analog are administered sequentially. In some specific aspects, the nucleoside analog is administered after the anthracycline is administered. In some of the foregoing embodiments, the anthracycline and the nucleoside analog are co-administered.

In some embodiments, a treatment regimen comprises 11 days of treatment. For example, some embodiments comprise administering the CDK inhibitor to the subject daily on a first, second and third day. Optionally, the subject receives no treatment (i.e., no CDK inhibitor, anthracycline, or nucleoside analog) on a fourth day. On a fifth day of treatment, embodiments include administering the nucleoside analog daily for 7 days (days 5-11) and administering the anthracycline daily for 3 days (days 5-7). In various embodiments, effective amounts of these therapeutic agents (e.g., CDK inhibitor, anthracycline, and/or nucleoside analog) can decrease the number of tumor cells, decrease the number of metastases, decrease tumor volume, induce apoptosis of cancer cells, induce cancer cell death, induce radio-sensitivity in cancer cells, inhibit angiogenesis near cancer cells, inhibit cancer cell proliferation, inhibit tumor growth, prevent metastasis, reduce the number of metastases, increase life expectancy, prolong a subject's life, reduce cancer-associated pain, and/or reduce relapse or re-occurrence of the cancer following treatment.

In embodiments, alvocidib, or a pharmaceutically acceptable salt thereof or a prodrug of the foregoing is administered to a subject on the first, second and third days of a treatment; daunorubicin or idarubicin, or a pharmaceutically acceptable salt of the foregoing, is administered to the subject on the fifth, sixth and seventh days of the treatment; and cytarabine, or a pharmaceutically acceptable salt thereof, is administered to the subject on the fifth, sixth, seventh, eighth, ninth, tenth and eleventh days of the treatment.

Embodiments including administering the nucleoside analog daily for seven days (e.g., on days 5-11 of a treatment) and administering the anthracycline daily for three days (e.g., on days 5-7 of the treatment) are referred to herein as “7+3” regimens.

In some embodiments of the methods disclosed herein, more than one cycle of the treatment is necessary. Accordingly, the methods disclosed herein can include one or more treatment cycles, e.g., 1, 2, 3, 4, 5, 6, 7, 8, 9, 10, 11, 12, etc. treatment cycles. In some embodiments, the methods disclosed herein include one treatment cycle, e.g., a treatment cycle wherein a CDK inhibitor is administered to a subject in combination with an anthracycline and a nucleoside analog in a 7+3 regimen. In some embodiments, the methods disclosed herein include two treatment cycles, e.g., a treatment cycle wherein a CDK inhibitor is administered to a subject in combination with an anthracycline and a nucleoside analog in a 7+3 regimen, and a treatment cycle wherein a CDK inhibitor is administered to a subject in combination with an anthracycline and a nucleoside analog in a 5+2 regimen.

The length of a treatment cycle is determined by the treatment being administered, but can be 1, 2, 3, 4, 5, 6, 7, 8, 9, 10, 11, 12, 13, 14, 15, 16, 17, 18, 19, 20, 21, 22, 23, 24, 25, 26, 27 or 28 days, or 4, 5, 6, 7, 8, 9, 10, 11 or 12 weeks. In embodiments wherein each treatment is 11 days in duration (e.g., as when a CDK inhibitor is administered to a subject in combination with an anthracycline and a nucleoside analog in a 7+3 regimen), the treatment cycle can be 14 days, meaning a new treatment cycle (e.g., a CDK inhibitor administered to a subject in combination with an anthracycline and a nucleoside analog in a 5+2 regimen) is initiated on the day corresponding to day 15 of the first treatment cycle. In embodiments wherein each treatment is 11 days in duration (e.g., as when a CDK inhibitor is administered to a subject in combination with an anthracycline and a nucleoside analog in a 7+3 regimen), the treatment cycle can be 28 days, meaning a new treatment cycle is initiated on the day corresponding to day 29 of the first treatment cycle. The appropriate length of a treatment cycle can be determined by a clinician skilled in the art.

Accordingly, in certain embodiments the disclosure provides a method for treating a cancer in a subject, the method comprising administering to the subject a treatment comprising an effective amount of:

alvocidib, or a prodrug thereof;

daunorubicin or idarubicin; and

cytarabine

thereby treating the cancer in the subject.

In certain embodiments the disclosure provides a method for treating a cancer in a subject, the method comprising administering to the subject a treatment comprising an effective amount of each of:

alvocidib, or a prodrug thereof, or a pharmaceutically acceptable salt of the foregoing;

daunorubicin or idarubicin, or a pharmaceutically acceptable salt of the foregoing; and

cytarabine, or a pharmaceutically acceptable salt thereof,

thereby treating the cancer in the subject.

In some embodiments, the method comprises administering to the subject a treatment comprising an effective amount of each of: alvocidib, or a prodrug thereof, or a pharmaceutically acceptable salt of the foregoing; daunorubicin, or a pharmaceutically acceptable salt thereof; and cytarabine, or a pharmaceutically acceptable salt thereof. In some embodiments, the method comprises administering to the subject a treatment comprising an effective amount of each of: alvocidib, or a pharmaceutically acceptable salt thereof daunorubicin or idarubicin, or a pharmaceutically acceptable salt of the foregoing; and cytarabine, or a pharmaceutically acceptable salt thereof. In some embodiments, the method comprises administering to the subject a treatment comprising an effective amount of each of: alvocidib, or a pharmaceutically acceptable salt thereof; daunorubicin, or a pharmaceutically acceptable salt thereof and cytarabine, or a pharmaceutically acceptable salt thereof. In some embodiments, the method comprises administering to the subject a treatment comprising an effective amount of each of: a prodrug of alvocidib, or a pharmaceutically acceptable salt thereof daunorubicin or idarubicin, or a pharmaceutically acceptable salt of the foregoing; and cytarabine, or a pharmaceutically acceptable salt thereof. In some embodiments, the method comprises administering to the subject a treatment comprising an effective amount of each of: a prodrug of alvocidib, or a pharmaceutically acceptable salt thereof; daunorubicin, or a pharmaceutically acceptable salt thereof; and cytarabine, or a pharmaceutically acceptable salt thereof.

In some embodiments the alvocidib, or prodrug thereof, daunorubicin or idarubicin, and cytarabine are administered sequentially. For example, some embodiments comprise administering the alvocidib, or prodrug thereof, to the subject before the daunorubicin or idarubicin, and the cytarabine.

Exemplary embodiments comprise 11 days of treatment. For example, some embodiments comprise administering the alvocidib, or prodrug thereof, to the subject daily on a first, second and third day. Optionally, the subject receives no treatment (i.e., no alvocidib, or prodrug thereof, daunorubicin or idarubicin, or cytarabine) on a fourth day. On a fifth day of treatment, embodiments include administering the cytarabine daily for 7 days (days 5-11) and administering the daunorubicin or idarubicin daily for 3 days (days 5-7). In various embodiments, effective amounts of these therapeutic agents (e.g., alvocidib, or prodrug thereof, daunorubicin or idarubicin, and/or cytarabine) can decrease the number of tumor cells, decrease the number of metastases, decrease tumor volume, induce apoptosis of cancer cells, induce cancer cell death, induce radio-sensitivity in cancer cells, inhibit angiogenesis near cancer cells, inhibit cancer cell proliferation, inhibit tumor growth, prevent metastasis, reduce the number of metastases, increase life expectancy, prolong a subject's life, reduce cancer-associated pain, and/or reduce relapse or re-occurrence of the cancer following treatment.

In particular embodiments, administration of the therapeutic agents of the disclosure (e.g., a CDK inhibitor and anthracycline) to a plurality of subjects results in an increase in one or more of progression free survival, complete remission rate, event free survival, overall survival, and bridge to bone marrow transplant.

In some embodiments, the treatment results in complete remission in the subject. A subject is considered to be in “complete remission” or “CR” if less than 5% leukemic blasts are present in the subject's bone marrow. Typically, a subject in CR has an absence of blasts and blasts with Auer rods, an absence of extramedullary disease, absolute neutrophil count ≥1.0×10⁹/L (1,000/μL) and platelet count of ≥100×10⁹/L (100,000/μL). As used herein, complete remission includes complete remission with partial hematological recovery (CRᵢ), meaning the subject meets the CR requirement for blast count, but exhibits residual neutropenia [<1.0×10⁹/L (1,000/μL)] or thrombocytopenia [<100×10⁹/L (100,000/μL)].

In some embodiments, administration of the therapeutic agents of the disclosure (e.g., a CDK inhibitor and anthracycline) to a subject results in negative measurable (minimal) residual disease (MRD) status.

Measurable residual disease, minimal residual disease and MRD refer to the post-therapy persistence of leukemic cells at levels below morphologic detection. Although not wishing to be bound by any particular theory, MRD is thought to be a strong prognostic indicator of increased risk of relapse or shorter survival in patients with hematologic cancers, such as AML. MRD testing is typically conducted using one of three techniques: immunophenotypic detection by multiparameter flow cytometry (MFC), real-time quantitative PCR (RT-qPCR) and next-generation sequencing technology. MFC uses panels of fluorochrome-labeled monoclonal antibodies to identify aberrantly expressed antigens of leukemic cells. RT-qPCR can be used to amplify leukemia-associated genetic abnormalities. Next-generation sequencing technology can be used to evaluate a few genes or an entire genome. Together, RT-qPCR and next-generation sequencing technology represent molecular approaches to MRD testing. Each of the foregoing methods of detecting MRD status in a subject is described in Ravandi, F., et al., Blood Advances 12 Jun. 2018, vol. 2, no. 11, and Schuurhuis, G. J., et al., Blood 2018 Mar. 22, 131(12): 1275-1291, the relevant contents of which are incorporated herein by reference in their entireties.

To guide the development of a standardized approach to MRD testing, the European LeukemiaNet (ELN) has issued consensus recommendations for the measurement of MRD in AML. According to the ELN, a percentage of cancer (e.g., AML) cells to leukocytes of 0.1% or greater in a subject's bone marrow, measured by MFC according to the ELN's recommendations for MRD testing by MFC, indicates the subject is MRD positive (MRD+) by MFC according to the ELN's recommendations for MRD testing by MFC. A percentage of cancer cells to leukocytes of less than 0.1% in a subject's bone marrow, measured by MFC according to the ELN's recommendations for MRD testing by MFC, indicates the subject is MRD negative (MRD−) by MFC according to the ELN's recommendations for MRD testing by MFC.

The ELN has also issued guidelines for molecular MRD testing. The ELN defines complete molecular remission as complete morphologic remission plus two successive negative MRD samples obtained within an interval of ≥4 weeks at a sensitivity level of at least 1 in 1,000, wherein the samples are collected and measured according to the ELN guidelines for molecular MRD testing. The ELN defines molecular persistence at low copy numbers, which is associated with a low risk of relapse, as MRD with low copy numbers (<100-200 copies/10⁴ ABL copies corresponding to <1-2% of target to reference gene or allele burden) in patients with morphologic CR, and a copy number or relative increase <1 log between any two positive samples collected at the end of treatment, wherein the samples are collected and measured according to the ELN guidelines for molecular MRD testing. The ELN defines molecular progression in patients with molecular persistence as an increase of MRD copy numbers ≥1 log 10 between any two positive samples collected and measured according to the ELN guidelines for molecular MRD testing. The ELN defines molecular relapse as an increase of the MRD level of ≥1 log 10 between two positive samples in a patient who previously tested negative, wherein the samples are collected and measured according to the ELN guidelines for molecular MRD testing. Both molecular persistence and molecular relapse are indicators of an MRD-positive subject by RT-qPCR conducted according to the ELN guidelines for MRD testing by RT-qPCR. Thus, patients in complete molecular remission and patients labelled as having molecular persistence at low copy numbers are MRD-negative by RT-qPCR conducted according to the ELN guidelines for MRD testing by RT-qPCR. The ELN does not currently recommend using next-generation sequencing to assess MRD status. Thus, RT-qPCR is the recommended molecular approach to MRD testing, as discussed in Ravandi, F., et al. and Schuurhuis, G. J., et al. Specific recommendations for collecting and measuring samples (e.g., bone marrow samples) for MRD testing are described in Ravandi, F., et al., Blood Advances 12 Jun. 2018, vol. 2, no. 11 and Schuurhuis, G. J., et al., Blood 2018 Mar. 22, 131(12): 1275-1291, the relevant contents of which are incorporated herein by reference in their entireties.

When a subject is described herein as being “measurable residual disease negative,” “minimal residual disease negative,” “MRD-negative” or “MRD⁻” without a further modifier, such as by MFC or by RT-qPCR, the subject is MRD negative according to at least one of the ELN's criteria described herein (e.g., MFC, molecular biology). In some embodiments, the subject is MRD-negative by MFC conducted according to ELN guidelines for MRD testing. In some embodiments, the subject is MRD-negative by RT-qPCR conducted according to ELN guidelines for MRD testing. In some embodiments, the subject is MRD-negative by both MFC and RT-qPCR conducted according to ELN guidelines for MRD testing. In some embodiments, the subject is MRD-negative by MFC conducted according to ELN guidelines for MRD testing, and is MRD-positive by RT-qPCR conducted according to ELN guidelines for MRD testing. In some embodiments, the subject is MRD-positive by MFC conducted according to ELN guidelines for MRD testing, and is MRD-negative by RT-qPCR conducted according to ELN guidelines for MRD testing. When a subject is MRD-negative according to one of the ELN's criterion described herein (e.g., the criterion for MFC), but MRD-positive according to another of the ELN's criterion described herein (e.g., the criterion for RT-qPCR), that subject can still be described as MRD-negative according to the use of that term herein because the subject is MRD negative according to at least one of the ELN's criteria described herein.

“Measurable residual disease positive,” “minimal residual disease positive,” “MRD-positive” or “MRD⁺,” refer to a subject that is MRD positive by the ELN's criteria for MFC and RT-qPCR described herein. For example, a subject that is MRD positive can be MRD-positive by MFC conducted according to ELN guidelines for MRD testing, and MRD-positive by RT-qPCR conducted according to ELN guidelines for MRD testing.

In some embodiments, negative MRD status means no detectable diseased cells are present in the subject's bone marrow.

Some embodiments of the methods described herein include administering the therapeutic agents described herein to the subject based on MRD status. Thus, some embodiments further comprise detecting the MRD status of the subject (e.g., prior to administering a CDK inhibitor and an anthracycline and, optionally, a nucleoside analog to the subject; after administering a CDK inhibitor and an anthracycline and, optionally, a nucleoside analog to the subject). In some embodiments, the MRD status of the subject is detected prior to administering the treatment to the subject. Detection of the subject's MRD status prior to treatment can be used to determine whether treatment with a CDK inhibitor and an anthracycline and, optionally, a nucleoside analog, is indicated, or whether a subsequent treatment is efficacious (e.g., as a baseline measurement). For example, detection of a positive MRD status in a subject may indicate that treatment according to a method disclosed herein is indicated, despite the subject being in complete remission. Alternatively, detection of a negative MRD status may indicate that the subject is not a suitable candidate for treatment according to a method disclosed herein.

In some embodiments, the MRD status of the subject is detected during the treatment and/or after administering the treatment to the subject. Detection of the subject's MRD status during or after treatment can be used, for example, to determine whether to terminate treatment or to track the progress of a subject on the treatment. For example, detection of a positive MRD status in a subject may indicate that continued treatment (e.g., another cycle of treatment) according to a method disclosed herein is indicated, despite the subject having completed a cycle of treatment. Alternatively, detection of a negative MRD status may indicate that treatment may be terminated.

In some embodiments, the MRD status of the subject is detected prior to and after administering the treatment to the subject.

Because MRD is believed to be a useful clinical endpoint, detection of MRD negative status in a subject is a useful clinical finding. Accordingly, in some embodiments of the methods described herein, the method further comprises terminating the treatment (e.g., with the CDK inhibitor and/or the anthracycline and/or the cytarabine) when detection of the subject's MRD status reveals that the subject is MRD-negative.

In embodiments, methods of the disclosure further comprise administering to the subject an effective amount of one or more additional treatment agents, such as one or more chemotherapeutic agents. In various embodiments, the one or more chemotherapeutic agents are one or more of alkylating agents, such as thiotepa or CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycin, dactinomycin, detorubicin, 6-diazo-5-oxo-L-norleucine, adriamycin doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C” or Cytarabine); cyclophosphamide; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chlorambucil; GEMZAR gemcitabine; 6-thioguanine; mercaptopurine; methotrexate; platinum analogs such as cisplatin, oxaliplatin and carboplatin; vinblastine; platinum; etoposide (VP-16); mitoxantrone; vincristine; NAVELBINE; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-1, Raf, H-Ras, EGFR (e.g., erlotinib (Tarceva)) and VEGF-A that reduce cell proliferation, Dacogen, Velcade, and pharmaceutically acceptable salts, acids or derivatives of any of the agents listed herein.

In embodiments, the one or more chemotherapeutic agents are selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that can be administered in conjunction with embodiments of the present methods include fluorouracil (5-FU) alone or in further combination with leukovorin; other pyrimidine analogs such as UFT, capecitabine, gemcitabine and cytarabine, the alkyl sulfonates, e.g., busulfan, improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil, cyclophosphamide, estramustine, ifosfamide, novembrichin, prednimustine and uracil mustard; and triazines, e.g., dacarbazine. In embodiments, the alkylating agent is thiotepa or CYTOXAN cyclosphosphamide.

Examples of antimetabolite chemotherapeutic agents that can be administered in conjunction with embodiments of the present methods include folic acid analogs, e.g., methotrexate and pteropterin; and the purine analogs such as mercaptopurine and thioguanine.

Examples of natural product-based chemotherapeutic agents that may be administered in conjunction with certain embodiments of the present method include the vinca alkaloids, e.g., vinblastine, vincristine and vindesine; the epipodophyllotoxins, e.g., etoposide and teniposide; the antibiotic chemotherapeutic agents, e.g., doxorubicin, epirubicin, mitomycin, dactinomycin, temozolomide, plicamycin, bleomycin; and the enzymatic chemotherapeutic agents such as L-asparaginase.

Examples of useful COX-II inhibitors include Vioxx, CELEBREX (celecoxib), valdecoxib, paracoxib, rofecoxib, and Cox 189.

Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/M98/01113 (filed Jul. 21, 1998), European Patent Application No. 99302232.1 (filed Mar. 25, 1999), Great Britain patent application number 9912961.1 (filed Jun. 3, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference for their teachings regarding the same. Preferred MMP-2 and MMP-9 inhibitors are those that have little or no activity inhibiting MMP-1. More preferred are those that selectively inhibit MMP-2 and/or MMP-9 relative to the other matrix-metalloproteinases MMP-3, MMP-4, MMP-5, MMP-6, MMP-7, MMP-8, MMP-10, MMP-11, MMP-12, and MMP-13).

Some specific examples of MMP inhibitors are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Embodiments of the method of treatment can also be combined with administration of signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example, WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), all of which are incorporated herein by reference in their entireties for their teachings regarding the same, and such substances can be used in the methods described herein.

EGFR-inhibiting agents include the monoclonal antibodies C225 and anti-EGFR 22Mab (ImClone Systems, Inc., New York, N.Y.), the compounds ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used as additional therapeutic agents in embodiments of the present disclosure. VEGF inhibitors, for example SU-5416 and SU-6668 (Sugen Inc., South San Francisco, Calif.), can also be combined with the therapeutic agents of the present disclosure. VEGF inhibitors are described in, for example, WO 01/60814 A3 (published Aug. 23, 2001), WO 99/24440 (published May 20, 1999), PCT International Application PCT/IB99/00797 (filed May 3, 1999), WO 95/21613 (published Aug. 17, 1995), WO 99/61422 (published Dec. 2, 1999), U.S. Pat. No. 5,834,504 (issued Nov. 10, 1998), WO 01/60814, WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference for their teachings regarding the same. Other examples of some specific VEGF inhibitors useful in the present disclosure are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present disclosure as described herein. pErbB2 receptor inhibitors, such as GW-282974 (Glaxo Wellcome plc), and the monoclonal antibodies AR-209 (Aronex Pharmaceuticals Inc., The Woodlands, Tex.) and 2B-1 (Chiron), can furthermore be combined with an inventive combination, for example, those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference for their teachings regarding the same. ErbB2 receptor inhibitors useful in the methods of this disclosure are also described in U.S. Pat. No. 6,284,764 (issued Sep. 4, 2001), incorporated in its entirety herein by reference for its teaching regarding the same. The erbB2 receptor inhibitor compounds and substance described in the aforementioned PCT applications, U.S. patents, and U.S. provisional applications, as well as other compounds and substances that inhibit the erbB2 receptor, can be used with the methods of the present disclosure.

In various embodiments, methods of the disclosure further comprise administering to the subject an effective amount of a pro-apoptotic agent and/or an agent that operates via apoptosis and/or an agent that operates via apoptosis driven by direct protein modulation. Examples of such agents include ABT-263 (Navitoclax), and obatoclax, WEP, bortezomib, and carfilzomib.

In any of the above embodiments, an effective amount of one or more of the following may also be administered to the subject: (i) a bromodomain inhibitor (e.g., a Brd2 inhibitor, a Brd3 inhibitor, a Brd4 inhibitor and/or a BrdT inhibitor); (ii) a histone methyltransferase inhibitor (e.g., a DOT1-like histone methyltransferase (Dot1L) inhibitor); (iii) a histone deacetylase (HDAC) inhibitor (e.g., a Class I HDAC (e.g., HDAC1, HDAC2, HDAC3 and HDAC8) inhibitor, a Class IIa HDAC (e.g., HDAC4, HDAC5, HDAC7, and HDAC9) inhibitor; a Class Hb HDAC (e.g., HDAC6 and HDAC10) inhibitor; and a Class IV HDAC (e.g., HDAC11) inhibitor); and (iv) a histone demethylase inhibitor (e.g. an inhibitor of a lysine-specific demethylase, such as lysine-specific demethylase 1A (Lsd1)).

In embodiments, an effective amount of one or more treatment agents that inhibit bromodomain proteins such as Brd2, Brd3, Brd4 and/or BrdT, is also administered to a subject. In some embodiments, the bromodomain inhibitor is a Brd4 inhibitor. In some embodiments, the bromodomain inhibitor is JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct. 10; 56(19):7498-500; GlaxoSmithKline), IBET151 (Bioorg. Med. Chem. Lett. 2012 Apr. 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium), or CPI-0610 (Constellation Pharmaceuticals).

In yet further embodiments, an effective amount of one or more treatment agents that inhibit histone methyltransferase proteins such as the DOT1-like histone methyltransferase (Dot1L) is also administered to a subject. In some embodiments, the histone methyltransferase inhibitor is EPZ004777, EPZ-5676 (Blood. 2013 Aug. 8; 122(6):1017-25) or SGC0946 (Nat. Commun. 2012; 3:1288). In specific embodiments, the histone methyltransferase inhibitor is EPZ-5676.

In embodiments, an effective amount of one or more treatment agents that inhibit histone deacetylase (HDAC) proteins is also administered to a subject. HDAC proteins may be grouped into classes based on homology to yeast HDAC proteins with Class I made up of HDAC1, HDAC2, HDAC3 and HDAC8; Class IIa made up of HDAC4, HDAC5, HDAC7 and HDAC9; Class IIb made up of HDAC6 and HDAC10; and Class IV made up of HDAC11. In some embodiments, the HDAC inhibitor is trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct. 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr. 13; 96(8):4592-7), mocetinostat (Bioorg. Med. Chem. Lett. 2008 Feb. 1; 18(3):1067-71) or valproic acid (EMBO J. 2001 Dec. 17; 20(24):6969-78). For example, in some embodiments, the HDAC inhibitor is panobinostat. In some embodiments, the HDAC inhibitor is panobinostat or SAHA.

In some embodiments, an effective amount of one or more treatment agents that inhibit histone demethylases, for example lysine-specific demethylases such as the lysine-specific demethylase 1A (Lsd1), is administered to a subject. In some embodiments, the histone demethylase inhibitor is HCl-2509 (BMC Cancer. 2014 Oct. 9; 14:752), tranylcypromine, or ORY-1001 (J. Clin. Oncol 31, 2013 (suppl; abstr e13543)).

In embodiments, methods of the disclosure further comprise administering to the subject an effective amount of a Brd4 inhibitor, a DNA methyltransferase inhibitor (e.g., azacitidine or decitabine), or both. In particular embodiments, methods of the disclosure further comprise administering to the subject an effective amount of a DNA methyltransferase inhibitor (e.g., azacitidine or decitabine).

In embodiments, an effective amount of an intercalation agent is also administered to the subject. In some such embodiments, the intercalation agent is mitoxantrone. In embodiments, an effective amount of a Bcl-2 inhibitor is also administered to the subject. In some such embodiments, the Bcl-2 inhibitor is venetoclax.

In embodiments, an effective amount of a fms-like tyrosine kinase 3 (FLT3) inhibitor is also administered to the subject. In some embodiments, the FLT3 inhibitor is midostaurin. In embodiments, an effective amount of an Isocitrate dehydrogenase (IDH)1 or IDH2 inhibitor is also administered to the subject. In some embodiments, the IDH1 or IDH2 inhibitor is gemtuzumab ozogamicin, enasidenib, or a combination thereof.

Administration of the therapeutic agents (e.g., a CDK inhibitor and anthracycline), in pure form or in an appropriate pharmaceutical composition, can be carried out via any of the accepted modes of administration of agents for serving similar utilities. Pharmaceutical compositions include at least one pharmaceutically acceptable carrier, diluent, or excipient and one or more therapeutic agents. The therapeutic agents are in free-acid or free-base form, or in a pharmaceutically acceptable salt form. In some embodiments, the therapeutic agents are present as prodrugs. In embodiments, the CDK inhibitor is a prodrug of alvocidib. Even if not specifically described in each instance, all embodiments which include alvocidib, optionally comprise use of a prodrug of alvocidib instead of, or in addition to, the parent compound. Such prodrugs are described in International Application No. PCT/US2016/033099, published as International Publication No. WO 2016/187316, which is incorporated herein by reference in its entirety for its teachings regarding the same. In embodiments, the CDK inhibitor is a phosphate prodrug of alvocidib. In certain embodiments, the phosphate prodrug of alvocidib has the following structure:

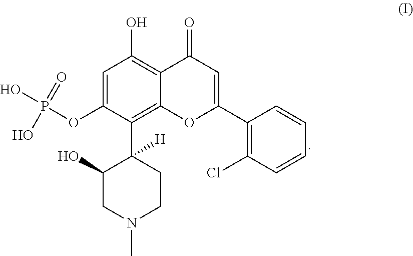 [SMILES: CN1CC[C@H](c2c(OP(=O)(O)O)cc(O)c3c(=O)cc(-c4ccccc4Cl)oc23)[C@H](O)C1]

The compound of Structural Formula I is orally bioavailable. Thus, the compound of Structural Formula I, or a pharmaceutically acceptable salt thereof, can be administered orally, and compositions comprising a compound of Structural Formula I, or a pharmaceutically acceptable salt thereof, can be formulated for oral administration.

In some embodiments, the compound of Structural Formula I, or a pharmaceutically acceptable salt thereof, is administered (e.g., orally) 1, 2, 3, 4, 5 or 6 times per day. In some embodiments, the compound of Structural Formula I, or a pharmaceutically acceptable salt thereof, is administered (e.g., orally) once daily. In some embodiments, the compound of Structural Formula I, or a pharmaceutically acceptable salt thereof, is administered (e.g., orally) twice daily. In some embodiments, the compound of Structural Formula I, or a pharmaceutically acceptable salt thereof, is administered (e.g., orally) three times daily.

In some embodiments, the compound of Structural Formula I is administered (e.g., orally) in an amount ranging from 1 mg/day to about 100 mg/day, from about 1 mg/day to about 50 mg/day, from about 5 mg/day to about 30 mg/day, from about 10 mg/day to about 20 mg/day, from about 15 mg/day to about 45 mg/day, or from about 20 mg/day to about 40 mg/day.

In some embodiments, the compound of Structural Formula I, or a pharmaceutically acceptable salt thereof, is administered (e.g., orally) daily for 1, 2, 3, 4, 5, 6, 7, 8, 9, 10, 11, 12, 13, 14, 15, 16, 17, 18, 19, 20, 21, 22, 23, 24, 25, 26, 27 or 28 days, or 4, 5, 6, 7, 8, 9, 10, 11 or 12 weeks. In a particular embodiment, the compound of Structural Formula I, or a pharmaceutically acceptable salt thereof, is administered (e.g., orally) daily for 14 days.

In some embodiments, the methods described herein comprise administering to a subject in need thereof a treatment comprising a prodrug, particularly an oral prodrug, of alvocidib, such as a compound of Structural Formula I, or a pharmaceutically acceptable salt thereof, and an anthracycline and a nucleoside analog in a 7+3 regimen. In some embodiments, the method comprises administering to the subject the anthracycline (e.g., daunorubicin or idarubicin, or a pharmaceutically acceptable salt of the foregoing) on the first, second and third days of the treatment; administering to the subject the nucleoside analog (e.g., cytarabine, or a pharmaceutically acceptable salt thereof) on the first, second, third, fourth, fifth, sixth, and seventh days of the treatment; and administering a prodrug of alvocidib (e.g., a compound of Structural Formula I), or a pharmaceutically acceptable salt thereof, to the subject on the eighth, ninth, tenth, eleventh, twelfth, thirteenth, fourteenth, fifteenth, sixteenth, seventeenth, eighteenth, nineteenth, twentieth and twenty-first days of the treatment. Typically, such a treatment will be carried out on a 28-day cycle, but other lengths of treatment cycle are contemplated. For example, a treatment cycle can be 1, 2, 3, 4, 5, 6, 7, 8, 9, 10, 11, 12, 13, 14, 15, 16, 17, 18, 19, 20, 21, 22, 23, 24, 25, 26, 27 or 28 days, or 4, 5, 6, 7, 8, 9, 10, 11 or 12 weeks in length. Such a treatment can also include one or more treatment cycles, e.g., 1, 2, 3, 4, 5, 6, 7, 8, 9, 10, 11, 12, etc. treatment cycles. In some embodiments, the method includes one, at least one, two, at least two, at least four, or at least 12 treatment cycles.

In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

The pharmaceutical compositions of the disclosure can be prepared by combining therapeutic agents (e.g., a CDK inhibitor and/or an anthracycline) with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the disclosure are formulated so as to allow the active ingredients contained therein to be bioavailable upon administration of the composition to a patient.

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or a like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When a pharmaceutical composition is in the form of a capsule, for example, a gelatin capsule, it may contain, in addition to materials of the above type, a liquid carrier such as polyethylene glycol or oil.

A pharmaceutical composition for use in the present methods may be in the form of a liquid, for example, an elixir, syrup, solution, emulsion or suspension. The liquid may be for oral administration or for delivery by injection, for example. When intended for oral administration, pharmaceutical compositions contain, for example in addition to the therapeutic compound(s), one or more of a sweetening agent, preservative, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions used in certain embodiments of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. Physiological saline is a preferred adjuvant. An injectable pharmaceutical composition is preferably sterile. In embodiments, the pharmaceutical composition is formulated for injection. In some embodiments, the pharmaceutical composition is formulated for bolus injection. In embodiments, the pharmaceutical composition is formulation for infusion.

A liquid pharmaceutical composition used in embodiments of the disclosure intended for either parenteral or oral administration should contain an amount of one or more of the therapeutic agents such that a suitable dosage will be obtained.

In certain embodiments, a therapeutic agent described herein is administered in a local rather than systemic manner, for example, via injection of the agent directly into an organ, often in a depot preparation or sustained release formulation. In specific embodiments, long acting formulations are administered by implantation (for example, subcutaneously or intramuscularly) or by intramuscular injection. Furthermore, in other embodiments, the therapeutic agent is delivered in a targeted drug delivery system, for example, in a liposome coated with organ-specific antibody. In such embodiments, the liposomes are targeted to and taken up selectively by the organ. In yet other embodiments, the therapeutic agent described herein is provided in the form of a rapid release formulation, in the form of an extended release formulation, or in the form of an intermediate release formulation.

A pharmaceutical composition to be used for certain embodiments of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

A pharmaceutical composition for use in certain embodiments of the disclosure (e.g., in solid or liquid form) may include an agent that binds to the therapeutic compound(s) and thereby assists in delivery. Suitable agents that may act in this capacity include a monoclonal or polyclonal antibody, a protein or a liposome.

Delivery systems for hydrophobic pharmaceutical compounds may be employed. Liposomes and emulsions are examples of delivery vehicles or carriers useful herein. In certain embodiments, organic solvents such as N-methylpyrrolidone are also employed. In additional embodiments, the therapeutic agents described herein are delivered using a sustained-release system, such as semipermeable matrices of solid hydrophobic polymers containing the therapeutic agent. Various sustained-release materials are useful herein. In some embodiments, sustained-release capsules release the therapeutic agents for a few weeks up to over 100 days. Depending on the chemical nature and the biological stability of the therapeutic agent, additional strategies for protein stabilization are employed.

In embodiments, two or more of the therapeutic agents (e.g., a CDK inhibitor and an anthracycline, or an anthracycline and a nucleoside analog) are formulated together in a liposomal formulation. In particular embodiments, such a liposomal formulation is Vyxeos.

A pharmaceutical composition used in certain embodiments of the disclosure may be prepared by methodology well known in the pharmaceutical art. For example, a pharmaceutical composition intended to be administered by injection can be prepared by combining one or more of the therapeutic agents (e.g., a CDK inhibitor and an anthracycline) with sterile, distilled water so as to form a solution. In some embodiments, pharmaceutical composition(s) for administration according to methods of the disclosure take the form of a liquid where the therapeutic agents are present in solution, in suspension, or both. In some embodiments, when a therapeutic agent is administered as a solution or suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of the therapeutic agents. The term “solubilizing agent” generally includes agents that result in formation of a micellar solution or a true solution of the agent. Certain acceptable nonionic surfactants, for example polysorbate 80, are useful as solubilizing agents, as are ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

A surfactant may be added to facilitate the formation of a homogeneous solution or suspension. Surfactants are compounds that non-covalently interact with the therapeutic compound(s) so as to facilitate dissolution or homogeneous suspension aqueous delivery system. In embodiments, a pharmaceutical composition includes one or more surfactants to enhance physical stability. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

A pharmaceutical composition for use in some embodiments of the disclosure may be intended for rectal administration, in the form, for example, of a suppository, which will melt in the rectum and release one or more of the therapeutic agents (e.g., a CDK inhibitor and an anthracycline). The composition for rectal administration may contain an oleaginous base as a suitable nonirritating excipient. Such bases include lanolin, cocoa butter and polyethylene glycol.

A pharmaceutical composition for use in embodiments of the disclosure may include various materials, which modify the physical form of a solid or liquid dosage unit. For example, the composition may include materials that form a coating shell around one or more of the therapeutic agents (e.g., a CDK inhibitor and an anthracycline). The materials that form the coating shell are typically inert, and may be selected from, for example, sugar, shellac, and other enteric coating agents. Alternatively, the active ingredients may be encased in a gelatin capsule.

A pharmaceutical composition used in certain embodiments may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of the therapeutic agents (e.g., a CDK inhibitor and an anthracycline) may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

In any of the foregoing embodiments, one or more of the therapeutic agents (e.g., a CDK inhibitor and an anthracycline), or their pharmaceutically acceptable salts, are administered in an effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Toxicity and therapeutic efficacy of methods described herein can be determined by standard pharmaceutical procedures in cell cultures or experimental animals, e.g., by determining the IC₅₀ and the LD₅₀ for an administered compound. For administration, effective amounts (also referred to as doses) can be initially estimated based on results from in vitro assays and/or animal model studies. For example, a dose can be formulated in animal models to achieve a circulating concentration range that includes an IC₅₀ as determined in cell culture against a particular target. The dosage may vary depending upon the dosage form employed and the route of administration utilized. The exact formulation, route of administration and dosage can be chosen by the individual physician in view of the patient's condition. (See, e.g., Goodman & Gilman's The Pharmacological Basis Of Therapeutics, Ch. 3, 9^{th} ed., Ed. by Hardman, J., and Limbard, L., McGraw-Hill, New York City, 1996, p. 46.)

Compositions that will be administered to a subject take the form of one or more dosage units, where for example, a tablet may be a single dosage unit, and a container of one or more therapeutic agents of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The pharmaceutical composition to be administered using certain embodiments of the methods of the disclosure will, in any event, contain an effective amount of one or more of the therapeutic agents (e.g., a CDK inhibitor and an anthracycline), or pharmaceutically acceptable salts thereof, for treatment of a cancer in accordance with the teachings of embodiments of this disclosure.

The therapeutic agents (e.g., CDK inhibitor and an anthracycline) described herein are effective over a wide dosage range. For example, in the treatment of adult humans, dosages from about 0.01 mg to about 1000 mg, from about 0.5 mg to about 100 mg, from about 1 mg to about 50 mg per day, and from about 5 mg to about 40 mg per day are examples of dosages that are used in some embodiments. An exemplary dosage is about 10 mg to about 30 mg per day.

In embodiments, a therapeutic agent is administered in a dose ranging from about 10 mg/m² to about 120 mg/m² per day. In embodiments, a therapeutic agent is administered in a dose ranging from about 10 mg/m² to about 100 mg/m² per day. In some embodiments, a therapeutic agent is administered in a dose ranging from about 45 mg/m² to about 90 mg/m² per day. In some embodiments, a therapeutic agent is administered in a dose ranging from about 30 mg/m² to about 60 mg/m² per day. In some embodiments, a therapeutic agent is administered in a dose ranging from about 15 mg/m² to about 35 mg/m² per day. In some embodiments, a therapeutic agent is administered in a dose ranging from about 20 mg/m² to about 30 mg/m² per day.

In certain embodiments, the dose of the CDK inhibitor (e.g., alvocidib, or a pharmaceutically acceptable salt thereof) ranges from about 10 mg/m² to about 120 mg/m² per day, from about 15 mg/m² to about 115 mg/m² per day, from about 10 mg/m² to about 100 mg/m² per day, from about 45 mg/m² to about 95 mg/m² per day, from about 50 mg/m² to about 90 mg/m² per day, or from about 25 mg/m² to about 60 mg/m² per day. In certain embodiments, the dose of the CDK inhibitor (e.g., alvocidib, or a pharmaceutically acceptable salt thereof) is about 50 mg/m² per day. In certain embodiments, the dose of the CDK inhibitor (e.g., alvocidib, or a pharmaceutically acceptable salt thereof) is about 90 mg/m² per day.

In certain embodiments, the dose of the anthracycline (e.g., daunorubicin, or a pharmaceutically acceptable salt thereof) ranges from about 30 mg/m² to about 110 mg/m² per day, from about 45 mg/m² to about 110 mg/m² per day, from about 45 mg/m² to about 90 mg/m² or from about 30 mg/m² to about 60 mg/m² per day. In certain embodiments, the dose of the anthracycline (e.g., daunorubicin, or a pharmaceutically acceptable salt thereof) is about 45 mg/m² per day. In certain embodiments, the dose of the anthracycline (e.g., daunorubicin, or a pharmaceutically acceptable salt thereof) is about 60 mg/m² per day. In certain embodiments, the dose of the anthracycline (e.g., daunorubicin, or a pharmaceutically acceptable salt thereof) is about 90 mg/m² per day.

In certain embodiments, the dose of the nucleoside analog (e.g., cytarabine, or a pharmaceutically acceptable salt thereof) ranges from about 90 mg/m² to about 210 mg/m² per day or from about 90 mg/m² to about 110 mg/m² per day. In certain embodiments, the dose of the nucleoside analog (e.g., cytarabine, or a pharmaceutically acceptable salt thereof) is about 100 mg/m² per day. In certain embodiments, the dose of the nucleoside analog (e.g., cytarabine, or a pharmaceutically acceptable salt thereof) is about 200 mg/m² per day.

In certain embodiments, the dose of the CDK inhibitor ranges from about 20 mg/m² to about 60 mg/m² per day. In certain embodiments, the dose of the anthracycline ranges from about 45 mg/m² to about 90 mg/m² per day. In certain embodiments, the dose of the nucleoside analog ranges from about 90 mg/m² to about 110 mg/m² per day.

In certain embodiments, the dose of the CDK inhibitor (e.g., alvocidib, or a pharmaceutically acceptable salt thereof) ranges from about 15 mg/m² to about 115 mg/m² (e.g., from about 45 mg/m² to about 95 mg/m², from about 50 mg/m² to about 90 mg/m²) per day; the dose of the anthracycline (e.g., daunorubicin, or a pharmaceutically acceptable salt thereof) ranges from about 45 mg/m² to about 110 mg/m² (e.g., from about 45 mg/m² to about 95 mg/m², about 60 mg/m²) per day; and the dose of the nucleoside analog (e.g., cytarabine, or a pharmaceutically acceptable salt thereof) ranges from about 90 mg/m² to about 110 mg/m² (e.g., about 100 mg/m²) per day. In certain embodiments, the CDK inhibitor is administered by intravenous bolus in a dose ranging from about 5 mg/m² to about 50 mg/m² (e.g., from about 20 mg/m² to about 30 mg/m²) per day, and by intravenous infusion in a dose ranging from about 10 mg/m² to about 65 mg/m² (e.g., 30 mg/m² to about 60 mg/m²) per day.

The exact dosage will depend upon the therapeutic agent, the route of administration, the form in which the compound is administered, the subject to be treated, physical and physiological factors including target, body weight, severity of condition, type of cancer, previous or concurrent therapeutic interventions, idiopathy of the subject, and the preference and experience of the attending physician.

In some embodiments, an effective amount of a therapeutic agent is administered in a single dose. Typically, such administration will be by injection, e.g., intravenous injection, in order to introduce the agent quickly. However, other routes are used as appropriate. A single dose of a compound of the disclosure may also be used for treatment of an acute condition.

In embodiments, a therapeutic agent is administered intravenously, e.g., via a bolus injection. In some embodiments, the bolus injection is from about 5 minutes to about 60 minutes, from about 10 minutes to about 60 minutes, from about 5 minutes to about 30 minutes, from about 15 minutes to about 45 minutes, from about 20 minutes to about 40 minutes or from about 10 minutes to about 20 minutes in duration. In some embodiments, the bolus injection is about 30 minutes in duration. In some embodiments, the bolus injection is about 15 minutes in duration.

In some embodiments, a CDK inhibitor (e.g., alvocidib, or a pharmaceutically acceptable salt thereof) is administered intravenously, e.g., via a bolus injection. In some embodiments, the bolus injection is from about 5 minutes to about 60 minutes, from about 10 minutes to about 60 minutes, from about 15 minutes to about 45 minutes or from about 20 minutes to about 40 minutes in duration. In some embodiments, the bolus injection is about 30 minutes in duration.

In some embodiments, an anthracycline (e.g., daunorubicin, or a pharmaceutically acceptable salt thereof) is administered intravenously, e.g., via a bolus injection. In some embodiments, the bolus injection is from about 5 minutes to about 60 minutes, from about 5 minutes to about 30 minutes or from about 10 minutes to about 20 minutes in duration. In some embodiments, the bolus injection is about 15 minutes in duration.

In embodiments, a therapeutic agent is administered intravenously, e.g., via intravenous infusion. In some embodiments, the intravenous infusion is from about 1 hour to about 28 hours, from about 20 hours to about 28 hours, from about 22 hours to about 26 hours, from about 3 hours to about 5 hours or from about 3.75 hours to about 4.25 hours in duration. In some embodiments, the intravenous infusion is about 4 hours in duration. In some embodiments, the intravenous infusion is about 24 hours in duration.

In embodiments, a CDK inhibitor (e.g., alvocidib, or a pharmaceutically acceptable salt thereof) is administered intravenously, e.g., via intravenous infusion. In some embodiments, the intravenous infusion is from about 3 hours to about 5 hours or from about 3.75 hours to about 4.25 hours in duration. In some embodiments, the intravenous infusion is about 4 hours in duration.

In embodiments, a nucleoside analog (e.g., cytarabine, or a pharmaceutically acceptable salt thereof) is administered intravenously, e.g., via intravenous infusion. In some embodiments, the intravenous infusion is from about 20 hours to about 28 hours or from about 22 hours to about 26 hours in duration. In some embodiments, the intravenous infusion is about 24 hours in duration.

In some embodiments, a CDK inhibitor (e.g., alvocidib, or a pharmaceutically acceptable salt thereof) is administered via a bolus injection of from about 10 minutes to about 60 minutes in duration; and an intravenous infusion of from about three hours to about five hours in duration. Typically, the intravenous infusion follows the bolus injection. For example, the bolus injection can be followed about 30 minutes later by the intravenous infusion. However, the bolus injection can also follow the intravenous infusion in certain embodiments.

In embodiments, a therapeutic agent is administered via a bolus injection in a dose ranging from about 15 mg/m² to about 35 mg/m² per day. In some embodiments, a therapeutic agent is administered via a bolus injection in a dose ranging from about 20 mg/m² to about 30 mg/m² per day. In some embodiments, a therapeutic agent is administered via intravenous infusion in a dose ranging from about 30 mg/m² to about 60 mg/m² per day. In embodiments, a therapeutic agent is administered via bolus injection in a dose ranging from about 45 mg/m² to about 90 mg/m² per day. In embodiments, a therapeutic agent is administered via intravenous infusion in a dose ranging from about 90 mg/m² to about 110 mg/m² per day.

In embodiments, a therapeutic agent is administered via bolus injection in a dose ranging from about 5 mg/m² to about 110 mg/m², from about 5 mg/m² to about 50 mg/m², from about 25 mg/m² to about 60 mg/m², from about 25 mg/m² to about 35 mg/m², from about 5 mg/m² to about 35 mg/m², from about 10 mg/m² to about 30 mg/m², from about 30 mg/m² to about 110 mg/m² per day, from about 45 mg/m² to about 110 mg/m² per day, from about 45 mg/m² to about 90 mg/m² or from about 30 mg/m² to about 60 mg/m² per day. In some embodiments, a therapeutic agent is administered via bolus injection in a dose of about 10 mg/m² per day. In some embodiments, a therapeutic agent is administered via bolus injection in a dose of about 20 mg/m² per day. In some embodiments, a therapeutic agent is administered via bolus injection in a dose of about 30 mg/m² per day. In some embodiments, a therapeutic agent is administered via bolus injection in a dose of about 45 mg/m² per day. In some embodiments, a therapeutic agent is administered via bolus injection in a dose of about 50 mg/m² per day. In some embodiments, a therapeutic agent is administered via bolus injection in a dose of about 60 mg/m² per day. In some embodiments, a therapeutic agent is administered via bolus injection in a dose of about 90 mg/m² per day.

In embodiments, a CDK inhibitor (e.g., alvocidib, or a pharmaceutically acceptable salt thereof) is administered via bolus injection in a dose ranging from about 5 mg/m² to about 110 mg/m², from about 5 mg/m² to about 50 mg/m², from about 25 mg/m² to about 60 mg/m², from about 25 mg/m² to about 35 mg/m², from about 5 mg/m² to about 35 mg/m² or from about 10 mg/m² to about 30 mg/m² per day. In some embodiments, a CDK inhibitor (e.g., alvocidib, or a pharmaceutically acceptable salt thereof) is administered via bolus injection in a dose of about 10 mg/m² per day. In some embodiments, a CDK inhibitor (e.g., alvocidib, or a pharmaceutically acceptable salt thereof) is administered via bolus injection in a dose of about 20 mg/m² per day. In some embodiments, a CDK inhibitor (e.g., alvocidib, or a pharmaceutically acceptable salt thereof) is administered via bolus injection in a dose of about 30 mg/m² per day. In some embodiments, a CDK inhibitor (e.g., alvocidib, or a pharmaceutically acceptable salt thereof) is administered via bolus injection in a dose of about 50 mg/m² per day.

In embodiments, an anthracycline (e.g., daunorubicin, or a pharmaceutically acceptable salt thereof) is administered via bolus injection in a dose ranging from about 5 mg/m² to about 110 mg/m², from about 30 mg/m² to about 110 mg/m² per day, from about 45 mg/m² to about 110 mg/m² per day, from about 45 mg/m² to about 90 mg/m² or from about 30 mg/m² to about 60 mg/m² per day. In some embodiments, an anthracycline (e.g., daunorubicin, or a pharmaceutically acceptable salt thereof) is administered via bolus injection in a dose of about 45 mg/m² per day. In some embodiments, an anthracycline (e.g., daunorubicin, or a pharmaceutically acceptable salt thereof) is administered via bolus injection in a dose of about 60 mg/m² per day. In some embodiments, an anthracycline (e.g., daunorubicin, or a pharmaceutically acceptable salt thereof) is administered via bolus injection in a dose of about 90 mg/m² per day.

In certain embodiments, a therapeutic agent is administered via intravenous infusion in a dose of from about 10 mg/m² to about 210 mg/m², from about 10 mg/m² to about 65 mg/m², from about 15 mg/m² to about 115 mg/m² per day, from about 90 mg/m² to about 210 mg/m² per day or from about 90 mg/m² to about 110 mg/m² per day. In certain embodiments, a therapeutic agent is administered via intravenous infusion in a dose of about 15 mg/m² per day. In certain embodiments, a therapeutic agent is administered via intravenous infusion in a dose of about 30 mg/m² per day. In certain embodiments, a therapeutic agent is administered via intravenous infusion in a dose of about 40 mg/m² per day. In certain embodiments, a therapeutic agent is administered via intravenous infusion in a dose of about 50 mg/m² per day. In certain embodiments, a therapeutic agent is administered via intravenous infusion in a dose of about 60 mg/m² per day. In certain embodiments, a therapeutic agent is administered via intravenous infusion in a dose of about 100 mg/m² per day. In certain embodiments, a therapeutic agent is administered via intravenous infusion in a dose of about 200 mg/m² per day.

In certain embodiments, a CDK inhibitor (e.g., alvocidib, or a pharmaceutically acceptable salt thereof) is administered via intravenous infusion in a dose of from about 10 mg/m² to about 65 mg/m². In certain embodiments, a CDK inhibitor (e.g., alvocidib, or a pharmaceutically acceptable salt thereof) is administered via intravenous infusion in a dose of about 15 mg/m² per day. In certain embodiments, a CDK inhibitor (e.g., alvocidib, or a pharmaceutically acceptable salt thereof) is administered via intravenous infusion in a dose of about 30 mg/m² per day. In certain embodiments, a CDK inhibitor (e.g., alvocidib, or a pharmaceutically acceptable salt thereof) is administered via intravenous infusion in a dose of about 40 mg/m² per day. In certain embodiments, a CDK inhibitor (e.g., alvocidib, or a pharmaceutically acceptable salt thereof) is administered via intravenous infusion in a dose of about 50 mg/m² per day. In certain embodiments, a CDK inhibitor (e.g., alvocidib, or a pharmaceutically acceptable salt thereof) is administered via intravenous infusion in a dose of about 60 mg/m² per day.

In certain embodiments, a nucleoside analog (e.g., cytarabine, or a pharmaceutically acceptable salt thereof) is administered via intravenous infusion in a dose of from about 90 mg/m² to about 210 mg/m² per day or from about 90 mg/m² to about 110 mg/m² per day. In certain embodiments, a nucleoside analog (e.g., cytarabine, or a pharmaceutically acceptable salt thereof) is administered via intravenous infusion in a dose of about 100 mg/m² per day. In certain embodiments, a nucleoside analog (e.g., cytarabine, or a pharmaceutically acceptable salt thereof) is administered via intravenous infusion in a dose of about 200 mg/m² per day.

In some embodiments, a CDK inhibitor is administered via a bolus injection in a dose ranging from about 20 mg/m² to about 30 mg/m² per day. In some embodiments, a CDK inhibitor is administered via intravenous infusion in a dose ranging from about 30 mg/m² to about 60 mg/m² per day. In embodiments, an anthracycline is administered via bolus injection in a dose ranging from about 45 mg/m² to about 90 mg/m² per day. In embodiments, a nucleoside analog is administered via intravenous infusion in a dose ranging from about 90 mg/m² to about 110 mg/m² per day.

In certain embodiments, alvocidib, or a prodrug thereof, is administered via a bolus injection in a dose ranging from about 20 mg/m² to about 30 mg/m² per day. In some embodiments, alvocidib, or a prodrug thereof, is administered via intravenous infusion in a dose ranging from about 30 mg/m² to about 60 mg/m² per day. In embodiments, daunorubicin is administered via bolus injection in a dose ranging from about 45 mg/m² to about 90 mg/m² per day. In embodiments, cytarabine is administered via intravenous infusion in a dose ranging from about 90 mg/m² to about 110 mg/m² per day.

In some embodiments, a CDK inhibitor (e.g., alvocidib, or a pharmaceutically acceptable salt thereof) is administered via bolus injection of from about 10 minutes to about 60 minutes (e.g., from about 15 minutes to about 45 minutes) in duration in a dose ranging from about 25 mg/m² to about 60 mg/m² (e.g., about 50 mg/m²) per day. In some embodiments, a CDK inhibitor (e.g., alvocidib, or a pharmaceutically acceptable salt thereof) is administered via bolus injection of from about 10 minutes to about 60 minutes (e.g., from about 15 minutes to about 45 minutes) in duration in a dose ranging from about 5 mg/m² to about 50 mg/m² (from about 25 mg/m² to about 35 mg/m²) per day.

In some embodiments, a CDK inhibitor (e.g., alvocidib, or a pharmaceutically acceptable salt thereof) is administered via intravenous infusion of from about 3 hours to about 5 hours (e.g., about four hours) in duration in a dose ranging from about 5 mg/m² to about 75 mg/m² (e.g., from about 10 mg/m² to about 65 mg/m²) per day.

In some embodiments, a CDK inhibitor (e.g., alvocidib, or a pharmaceutically acceptable salt thereof) is administered via bolus injection of from about 10 minutes to about 60 minutes (e.g., from about 15 minutes to about 45 minutes) in duration in a dose ranging from about 5 mg/m² to about 50 mg/m² (from about 25 mg/m² to about 35 mg/m²) per day, and intravenous infusion of from about 3 hours to about 5 hours (e.g., about four hours) in duration in a dose ranging from about 5 mg/m² to about 75 mg/m² (e.g., from about 10 mg/m² to about 65 mg/m²) per day.

In some embodiments, a CDK inhibitor (e.g., alvocidib, or a pharmaceutically acceptable salt thereof) is administered by bolus injection of from about 10 minutes to about 60 minutes in duration in a dosage of from about 5 mg/m² to about 50 mg/m² per day, and intravenous infusion of about 4 hours in duration in a dosage of from about 10 mg/m² to about 65 mg/m² per day; an anthracycline (e.g., daunorubicin, or a pharmaceutically acceptable salt thereof) is administered by bolus injection of from about 5 minutes to about 30 minutes in duration in a dosage of from about 45 mg/m² to about 110 mg/m² per day; and a nucleoside analog (e.g., cytarabine, or a pharmaceutically acceptable salt thereof, is administered by intravenous infusion of from about 20 hours to about 28 hours in duration in a dosage of from about 90 mg/m² to about 110 mg/m² per day.

When alvocidib, or a pharmaceutically acceptable salt is administered by bolus injection and intravenous infusion, the bolus injection typically precedes the intravenous infusion. In some embodiments, an intravenous infusion of alvocidib, or a pharmaceutically acceptable salt thereof, is initiated within about one hour (e.g., within about 45 minutes, within about 30 minutes) of completion of the bolus injection of alvocidib, or a pharmaceutically acceptable salt thereof. In some embodiments, an intravenous infusion of alvocidib, or a pharmaceutically acceptable salt thereof, is initiated about 30 minutes after completion of a bolus injection of alvocidib, or a pharmaceutically acceptable salt thereof.

In both the 7+3 and 5+2 regimens, administration of daunorubicin, or a pharmaceutically acceptable salt thereof, typically precedes administration of cytarabine, or a pharmaceutically acceptable salt thereof. In some embodiments, an intravenous infusion of cytarabine, or a pharmaceutically acceptable salt thereof, is initiated within about one hour (e.g., within about 30 minutes, within about 15 minutes, within about 10 minutes, within about 5 minutes) of completion of a bolus injection of daunorubicin, or a pharmaceutically acceptable salt thereof. In some embodiments, an intravenous infusion of cytarabine, or a pharmaceutically acceptable salt thereof, is initiated immediately after completion of a bolus injection of daunorubicin, or a pharmaceutically acceptable salt thereof.

In some embodiments, an effective amount of a therapeutic agent is administered in multiple doses. In some embodiments, dosing is about once, twice, three times, four times, five times, six times, or more than six times per day. In other embodiments, dosing is about once a month, once every two weeks, once a week, or once every other day. In another embodiment, a first therapeutic agent and a second therapeutic agent are administered together about once per day to about 6 times per day. In another embodiment, administration of a first therapeutic agent and a second therapeutic agent continues for less than about 7 days. In yet another embodiment, the administration continues for more than about 6, 10, 14, 28 days, two months, six months, or one year. In some cases, continuous dosing is achieved and maintained as long as necessary.

Administration of the therapeutic agents may continue as long as necessary. In some embodiments, a therapeutic agent is administered for more than 1, 2, 3, 4, 5, 6, 7, 14, or 28 days. In some embodiments, a therapeutic agent is administered for less than 28, 14, 7, 6, 5, 4, 3, 2, or 1 day. In some embodiments, a therapeutic agent is administered chronically on an ongoing basis, e.g., for the treatment of chronic effects.

In some embodiments, one or more therapeutic agents are administered as a treatment regimen, followed by administration of an additional one or more therapeutic agents. In some such embodiments, the additional one or more therapeutic agents may be administered chronically (e.g., as a maintenance therapy). In other such embodiments, the additional one or more therapeutic agents may be administered as a second treatment regimen.

In specific embodiments, a CDK inhibitor is administered to a subject in combination with an anthracycline and a nucleoside analog in a 7+3 regimen. If such treatment does not result in complete remission, the anthracycline and the nucleoside analog may be administered in a further 5+2 regimen. A “5+2 regimen” is a regimen in which the nucleoside analog is administered daily for five days (e.g., days 12-17) and the anthracycline is administered daily for two days (e.g., days 12-13).

If treatment with a CDK inhibitor (e.g., alvocidib, or a pharmaceutically acceptable salt thereof, or prodrug of the foregoing) in combination with an anthracycline (e.g., daunorubicin or idarubicin, or a pharmaceutically acceptable salt of the foregoing) and a nucleoside analog (e.g., cytarabine, or a pharmaceutically acceptable salt thereof) in a 7+3 regimen does not result in complete remission, the CDK inhibitor can be administered to the subject in combination with the anthracycline and the nucleoside analog in a further 5+2 reinduction regimen. Accordingly, some embodiments of the methods described herein comprise a first treatment and a second treatment. The CDK inhibitor (e.g., alvocidib, or a prodrug thereof, or a pharmaceutically acceptable salt of the foregoing) is administered to the subject on the first, second and third days of the first treatment; the anthracycline (e.g., daunorubicin or idarubicin, or a pharmaceutically acceptable salt of the foregoing) is administered to the subject on the fifth, sixth and seventh days of the first treatment; and the nucleoside analog (e.g., cytarabine, or a pharmaceutically acceptable salt thereof) is administered to the subject on the fifth, sixth, seventh, eighth, ninth, tenth, and eleventh days of the first treatment. Optionally, there is a treatment break on the fourth day of the first treatment. The CDK inhibitor (e.g., alvocidib, or a prodrug thereof, or a pharmaceutically acceptable salt of the foregoing) is administered to the subject on the first, second and third days of the second treatment; the anthracycline (e.g., daunorubicin or idarubicin, or a pharmaceutically acceptable salt of the foregoing) is administered to the subject on the fifth and sixth days of the second treatment; and the nucleoside analog (e.g., cytarabine, or a pharmaceutically acceptable salt thereof) is administered to the subject on the fifth, sixth, seventh, eighth and ninth days of the second treatment. Optionally, there is a treatment break on the fourth day of the second treatment.

When a method described herein comprises first and second treatments, the second treatment can begin immediately, such that day one of the second treatment corresponds to day 12 of the first treatment, or the first and second treatments can be separated in time (e.g., by 1, 2, 3, 4, 5, 6, 7, etc. days, such that day one of the second treatment corresponds to day 13, 14, 15, 16, 17, 18, 19, etc., respectively, of the first treatment. In some embodiments, the first and second treatments are separated in time by 3 days, such that day one of the second treatment corresponds to day 15 of the first treatment.

In some embodiments, the CDK inhibitor (e.g., alvocidib, or a pharmaceutically acceptable salt thereof) is administered in a dosage of from about 10 mg/m² to about 100 mg/m² (e.g., from about 50 mg/m² to about 90 mg/m²; about 50 mg/m²; about 90 mg/m²) per day during the first treatment and/or second treatment.

In some embodiments, the anthracycline (e.g., daunorubicin or idarubicin, or a pharmaceutically acceptable salt of the foregoing) is administered in a dosage of from about 45 mg/m² to about 110 mg/m² (e.g., about 60 mg/m²) per day during the first treatment, and/or administered in a dosage of from about 30 mg/m² to about 60 mg/m² (e.g., about 45 mg/m²) per day during the second treatment.

In some embodiments, the nucleoside analog (e.g., cytarabine, or a pharmaceutically acceptable salt thereof) is administered in a dosage of from about 90 mg/m² to about 110 mg/m² (e.g., about 100 mg/m²) per day during the first treatment and/or the second treatment.

In some embodiments, the CDK inhibitor (e.g., alvocidib, or a pharmaceutically acceptable salt thereof) is administered intravenously, e.g., via a bolus injection of from about 10 minutes to about 60 minutes in duration; via intravenous infusion of from about 3 hours to about 5 hours in duration; or via a bolus injection of from about 10 minutes to about 60 minutes in duration, and (e.g., followed by) intravenous infusion of from about 3 hours to about 5 hours in duration, during the first treatment and/or second treatment.

In some embodiments, the anthracycline (e.g., daunorubicin or idarubicin, or a pharmaceutically acceptable salt of the foregoing) is administered intravenously, e.g., via a bolus injection of from about 5 minutes to about 30 minutes in duration during the first treatment and/or second treatment.

In some embodiments, the nucleoside analog (e.g., cytarabine, or a pharmaceutically acceptable salt thereof) is administered intravenously, e.g., via intravenous infusion of from about 20 hours to about 28 hours in duration during the first treatment and/or second treatment.

In some embodiments, the CDK inhibitor (e.g., alvocidib, or a pharmaceutically acceptable salt thereof) is administered via bolus injection of from about 10 minutes to about 60 minutes (e.g., from about 15 minutes to about 45 minutes) in duration in a dosage of from about 25 mg/m² to about 60 mg/m² (e.g., about 50 mg/m²) per day during the first treatment and/or second treatment. In some embodiments, the CDK inhibitor (e.g., alvocidib, or a pharmaceutically acceptable salt thereof) is administered via bolus injection of from about 10 minutes to about 60 minutes (e.g., from about 15 minutes to about 45 minutes) in duration in a dosage of from about 5 mg/m² to about 50 mg/m² (e.g., from about 25 mg/m² to about 35 mg/m²) per day during the first treatment and/or second treatment.

In some embodiments, the CDK inhibitor (e.g., alvocidib, or a pharmaceutically acceptable salt thereof) is administered via intravenous infusion of from about 3 hours to about 5 hours (e.g., about 4 hours) in duration in a dosage of from about 5 mg/m² to about 75 mg/m² (e.g., from about 10 mg/m² to about 65 mg/m²) per day during the first treatment and/or second treatment.

In some embodiments, the CDK inhibitor (e.g., alvocidib, or a pharmaceutically acceptable salt thereof) is administered via bolus injection of from about 10 minutes to about 60 minutes (e.g., from about 15 minutes to about 45 minutes) in duration in a dosage of from about 5 mg/m² to about 50 mg/m² (e.g., from about 25 mg/m² to about 35 mg/m²) per day during the first treatment and/or second treatment; and intravenous infusion of from about 3 hours to about 5 hours (e.g., about 4 hours) in duration in a dosage of from about 5 mg/m² to about 75 mg/m² (e.g., from about 10 mg/m² to about 65 mg/m²) per day during the first treatment and/or second treatment.

Alternative dosages and routes of administration, and alternative combinations of dosages and routes of administration involving the CDK inhibitor, the anthracycline and the nucleoside analog in methods comprising first and second treatments are as provided herein with respect to the methods in general.

In some embodiments, the therapeutic agents are administered in dosages. Due to intersubject variability in compound pharmacokinetics, individualization of dosing regimen is provided in certain embodiments. Dosing for a therapeutic agent may be found by routine experimentation in light of the instant disclosure and/or can be derived by one of ordinary skill in the art.

Dosage amount and interval may be adjusted individually to provide plasma levels of the active species which are sufficient to maintain desired pharmacological effects. These plasma levels are referred to as minimal effective concentrations (MECs). Dosages necessary to achieve the MEC will depend on individual characteristics and route of administration. HPLC assays or bioassays can be used to determine plasma concentrations.

Dosage intervals can also be determined using MEC values. In some embodiments, methods of treatment comprise maintaining plasma levels above the MEC for 10-90% of the time. In some embodiments, plasma levels are maintained between 30-90%. In some embodiments, plasma levels are maintained between 50-90%. For example, in certain embodiments, effective amounts of a therapeutic agent may range from approximately 2.5 mg/m² to approximately 1500 mg/m² per day. Additional illustrative amounts range from 0.2-1000 mg/qid, 2-500 mg/qid, and 20-250 mg/qid.

In cases of local administration or selective uptake, the effective local concentration of the therapeutic agent may not be related to plasma concentration, and other procedures known in the art may be employed to determine the correct dosage amount and interval.

In some embodiments, the concentration of one or more therapeutic agents provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more therapeutic agents provided in the pharmaceutical compositions is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in the pharmaceutical compositions is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in the pharmaceutical compositions is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

Each therapeutic agent (e.g., a CDK inhibitor and an anthracycline) used in embodiments of the disclosure, or pharmaceutically acceptable derivatives thereof, may also be administered simultaneously with, prior to, or after administration of one or more other therapeutic agents. For example, a first therapeutic agent can be administered and after a sufficient period of time a second therapeutic agent is administered. In such embodiments, the period of time between the administration of the first therapeutic agent and the second therapeutic agent may be referred to as a “treatment break.” In some embodiments, such a treatment break ranges from about 12 hours to about 48 hours. In some embodiments, such a treatment break ranges from about 18 to about 40 hours. In some embodiments, such a treatment break ranges from about 18 to about 36 hours. In some embodiments, such a treatment break ranges from about 24 to about 48 hours. One of ordinary skill in the art can derive an appropriate dosing schedule based on common techniques and knowledge.

In embodiments, at least two of the therapeutic agents (e.g., a CDK inhibitor and an anthracycline) are administered sequentially. In some such embodiments, the CDK inhibitor and the anthracycline are administered sequentially. In some embodiments, the anthracycline is administered after the CDK inhibitor is administered. In some embodiments, there is a treatment break between administering the CDK inhibitor and administering the anthracycline. In certain embodiments, the CDK inhibitor is administered to the subject prior to administration of the anthracycline. In more specific embodiments, the CDK inhibitor is administered within about 24 to about 48 hours before administration of the anthracycline. In more specific embodiments, the CDK inhibitor is administered more than about 24 to about 48 hours before administration of the anthracycline. In other embodiments, the anthracycline is administered the subject prior to administration of the CDK inhibitor.

In embodiments, methods of the present disclosure further comprise administering an effective amount of a nucleoside analog to the subject. In some embodiments, the nucleoside analog is a pyrimidine analog. Any suitable pyrimidine analog may be administered, such as UFT, capecitabine, gemcitabine and cytarabine, the alkyl sulfonates, e.g., busulfan, improsulfan and piposulfan. In particular embodiments, the pyrimidine analog is cytarabine. In certain embodiments, the CDK inhibitor is alvocidib, or a prodrug thereof, and the pyrimidine analog is cytarabine.

In embodiments, the anthracycline and the nucleoside analog are administered sequentially. In some embodiments, the nucleoside analog is administered after the anthracycline is administered. In embodiments, the CDK inhibitor, the anthracycline, and the nucleoside analog are administered sequentially. In some embodiments, the nucleoside analog is administered after the anthracycline, which is administered after the CDK inhibitor. In some embodiments, the administration of the nucleoside analog and the anthracycline overlaps. In some embodiments, the anthracycline is co-administered with the nucleoside analog. In some such embodiments, the nucleoside analog is also administered after the course of anthracycline is administered.

In embodiments, the CDK inhibitor, the anthracycline, and/or any other therapeutic agent administered to the subject are formulated as one or more pharmaceutical compositions. In some embodiments, the CDK inhibitor and the anthracycline are formulated into separate pharmaceutical compositions. In some embodiments, each therapeutic agent (i.e., the CDK inhibitor, the anthracycline, and/or any other therapeutic agent) is formulated separately.

A wide variety of cancers, including solid tumors and leukemias (e.g., acute myeloid leukemia) are amenable to the methods disclosed herein. In embodiments, the cancer is a solid cancer. In some embodiments, the cancer comprises a solid tumor. In embodiments, the cancer comprises a colorectal, breast, prostate, lung, pancreatic, renal, or ovarian tumor. In embodiments, the cancer is a non-solid cancer. In various embodiments, the cancer is a pre-metastatic cancer. In various embodiments, the cancer is a metastatic cancer.

Types of cancer that may be treated in various embodiments include: adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

In various embodiments, the cancer is acute lymphoblastic leukemia (ALL), acute myeloid leukemia (AML), adrenocortical carcinoma, AIDS-related cancers, anal cancer, appendix cancer, astrocytoma (e.g., childhood cerebellar or cerebral), basal-cell carcinoma, bile duct cancer, bladder cancer, bone tumor (e.g., osteosarcoma, malignant fibrous histiocytoma), brainstem glioma, brain cancer, brain tumors (e.g., cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumors, central nervous system lymphomas, cerebellar astrocytoma, cervical cancer, chronic lymphocytic leukemia (CLL), chronic myelogenous leukemia (CML), chronic myeloproliferative disorders, colon cancer, cutaneous t-cell lymphoma, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g., extracranial, extragonadal, ovarian), gestational trophoblastic tumor, gliomas (e.g., brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, leukemias (e.g., acute lymphocytic leukemia, acute myelogenous leukemia, chronic lymphocytic leukemia, chronic myeloid leukemia, hairy cell), lip and oral cavity cancer, liposarcoma, liver cancer, lung cancer (e.g., non-small cell, small cell), lymphoma (e.g., AIDS-related, Burkitt, cutaneous T-cell Hodgkin, non-Hodgkin, primary central nervous system), medulloblastoma, melanoma, Merkel cell carcinoma, mesothelioma, metastatic squamous neck cancer, mouth cancer, multiple endocrine neoplasia syndrome, multiple myeloma, mycosis fungoides, myelodysplastic syndromes, myelodysplastic/myeloproliferative diseases, myelogenous leukemia, myeloid leukemia, myeloid leukemia, myeloproliferative disorders, chronic, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, retinoblastoma, rhabdomyosarcoma, salivary gland cancer, sarcoma (e.g., Ewing family, Kaposi, soft tissue, uterine), Sézary syndrome, skin cancer (e.g., nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, or Wilms tumor.

In any of the treatment methods described herein, the cancer is a hematologic cancer. In some embodiments, the hematologic cancer is multiple myeloma, myelodysplastic syndrome (MDS), acute myeloid leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma. In specific embodiments, the cancer is multiple myeloma, AML, acute lymphocytic leukemia, chronic lymphogenous leukemia, mantle cell lymphoma, diffuse large B-cell lymphoma, and non-Hodgkin's lymphoma. In embodiments of the foregoing, the hematologic cancer is CLL.

In embodiments, the hematologic cancer is AML. In some embodiments, the hematologic cancer is AML with dysplastic features. In some embodiments, the AML is frontline, newly diagnosed AML. In some embodiments, the AML is not acute promyelocytic leukemia. In some embodiments, the AML is not core binding factor (CBF)-AML (i.e., the subject is not positive for CBF and the subject has t[8;21] or inv[16] or t[16;16] cytogenetics). In some embodiments, the subject has intermediate-risk or high-risk status according to the National Comprehensive Cancer Network (NCCN) guidelines. In some embodiments, the subject has high-risk status according to the NCCN guidelines. In some embodiments, a subject has intermediate-risk or high-risk status if positive for one or more of the following: Chromosome-5/-7 (monosomies of chromosome 5 and/or 7); Inv(3); t(3;3); t(6;9); t(9;22); 17p abnormalities (p53 gene); 11q23 abnormalities (MLL gene); 20q deletions; 3 or more abnormalities in the same patient; FLT3 mutations (single amino acid substitution or ITD); IDH1 or IDH2 mutations; TET2 mutations; RUNX mutations; ASXL1 mutations; PHF6 mutations; and DNMT3A mutations.

“Frontline, newly diagnosed,” used to describe a cancer herein, such as AML, means that the cancer has not previously been treated with a traditional frontline therapy, such as radiation, surgery or chemotherapy. Accordingly, in some embodiments of the methods described herein, the cancer (e.g., hematologic cancer, such as AML) is previously untreated.

The NCCN stratifies AML patients by genetic abnormality. Subjects having low-risk AML or whose AML is deemed to fall within the favorable risk category are those positive for core binding factor: inv(16) or t(16;16) or t(8;21) or t(15;17), except the presence of KIT mutations in subjects with t(8;21) and, to a lesser extent, inv(16) confers a high risk of relapse, and warrants classification of the subject's AML as intermediate-risk; and/or those with normal cytogenetics: NPM1 mutation in absence of FLT3-ITD or isolated biallelic (double) CEBPA mutation. In some embodiments of the methods described herein, the AML is low-risk AML. In some embodiments, the AML is favorable risk AML according to the NCCN guidelines.

Subjects having intermediate-risk AML or whose AML is deemed to fall within the intermediate risk category are those with normal cytogenetics, trisomy 8 alone, and those positive for t(9;11) and/or core binding factor with KIT mutation. In some embodiments of the methods described herein, the AML is intermediate-risk AML. In some embodiments, the AML is intermediate risk AML according to the NCCN guidelines.

Subjects having high-risk AML or whose AML is deemed to fall within the poor/adverse risk category are those positive for >3 clonal chromosomal abnormalities, monoclonal karyotype, -5, 5q-, -7, 7q-, 11q23-non t(9;11), inv(3), t(3;3), t(6;9), t(9;22), normal cytogenetics with FLT3-ITD mutation and/or TP53 mutation. In some embodiments of the methods described herein, the AML is high-risk AML. In some embodiments, the AML is poor/adverse-risk AML according to the NCCN guidelines.

There are other factors that can affect treatment outcomes in subjects being treated for cancer (e.g., a hematologic cancer, such as AML). For example, age, fitness for chemotherapy and MCL-1 dependence have all been linked to treatment outcomes in subject being treated for cancer.

Accordingly, in some embodiments of the methods disclosed herein, the subject is young (i.e., aged less than 60 years). In some embodiments, the subject is elderly (i.e., aged 60 years or more).

In some embodiments, the subject is fit. In some embodiments, the subject is unfit. As used herein, “unfit” refers to having one or more physiological impairments that render a subject ineligible for a certain treatment (e.g., standard-of-care chemotherapy, intensive chemotherapy, non-intensive chemotherapy). Some have taken a consensus-based approach to determining fitness of a subject. See, for example, Ferrara, F., et al., Leukemia (2013) 27, 997-999, the relevant teachings of which are incorporated herein by reference in their entireties. In some embodiments, fitness may be determined by the consensus approach put forth in Ferrara, F., et al. In such embodiments, unfitness to intensive chemotherapy means fulfillment of at least one of nine criteria identified in Ferrara, F., et al., and unfitness to non-intensive chemotherapy means fulfillment of at least one of six criteria identified in Ferrara, F., et al. The Eastern Cooperative Oncology Group (ECOG) has put forth the ECOG Performance Status, which is a tool used to quantify the functional status of cancer patients on a scale of 0-5. In some embodiments, fitness may be determined by the ECOG Performance Status. In some embodiments, an ECOG score of greater than or equal to 2 (e.g., greater than 2) renders a subject “unfit.”

A “fit” subject is a subject that is not unfit.

In some embodiments, the subject is young and/or fit. In some embodiments, the subject is elderly and/or unfit.

In some embodiments, the cancer (e.g., hematologic cancer, in particular, AML) is MCL-1 dependent AML. As used herein, “MCL-1-dependent AML” refers to the subset of AML wherein myeloid cell leukemia 1 (MCL-1) is the primary driver of suppressing apoptosis. Typically, MCL-1 dependency promotes AML blast survival, and is associated with treatment resistance and relapse. MCL-1 dependence can be assessed, for example, by contacting a subject's cancer cell with a profiling peptide, as described herein.

Although not wishing to be bound by any particular theory, it is thought that MCL-1 dependence is found in both MRD cells and leukemia stem cells (LSCs), those cells thought to be responsible for relapse in subjects and to play a role in refractory disease. Research shows that knockout of MCL-1 in mice results in loss of early bone marrow progenitor cell populations, suggesting that MCL-1 is the primary survival signal in hematopoietic stem cells. Opferman, J. T., et al., “Obligate Role of Anti-Apoptotic MCL-1 in the Survival of Hematopoietic Stem Cells,” Science, vol. 307, 18 Feb. 2005, the relevant contents of which are incorporated herein in their entireties. MCL-1 has also been identified as the main survival mechanism in LSCs from FLT3 positive AML. Yoshimoto, G., et al., “FLT3-ITD up-regulates MCL-1 to promote survival of stem cells in acute myeloid leukemia via FLT3-ITD-specific STATS activation,” Blood, vol. 114, no. 24, 3 Dec. 2009, the relevant contents of which are incorporated herein in their entireties. It is likely that all LSCs, including non-FLT3-positive LSCs, use a similar MCL-1-dependent survival mechanism as that observed in both hematopoietic stem cells generally and FLT3-positive LSCs.

Leukemia stem cells and MRD cells are not completely synonymous with one another. However, the MRD cells that ultimately lead to relapsed disease are leukemia stem cells. See Al-Malawi, A., “Leukemic Stem Cells Shows the Way for Novel Target of Acute Myeloid Leukemia Therapy,” J. Stem Cell Res. Ther., vol. 3, issue 4; Yanagisawa, B., et al., “Translating leukemia stem cells into the clinical setting: Harmonizing the heterogeneity,” Experimental Hematology 2016; 44: 1130-1137; and Gerber, J. M., et al., “A clinically relevant population of leukemic CD34⁺CD38” cells in acute myeloid leukemia,” Blood, 12 Apr. 2012, vol. 119, no. 15, the relevant contents of which are incorporated herein in their entireties. Without wishing to be bound by any particular theory, it is thus thought that MCL-1 regulation may be a rational therapeutic strategy for cancer (e.g., a hematologic cancer, such as AML).

Cyclin-dependent kinases, or CDKs, are a family of proteins that form complexes involved in either cell cycle progression or transcription regulation. CDK9 is a transcription-regulating CDK that promotes the expression of MCL-1 by phosphorylating the carboxyl-terminal domain of the largest subunit of RNA polymerase II, allowing transcriptional elongation of MCL-1 mRNA. Inhibition of CDK9, as by a CDK9 inhibitor such as alvocidib, is thus thought to provide the MCL-1 regulation that, in combination with one or more additional therapeutic agents (e.g., an anthracycline, such as daunorubicin or idarubicin and, optionally, a nucleoside analog, such as cytarabine) could be used to eliminate or substantially eliminate MCL-1-dependent cells, such as MRD cells and LSCs, thereby converting a subject from MRD-positive status to MRD-negative status, for example, to treat a cancer (e.g., a hematologic cancer, such as AML) and/or reduce risk of relapse in a subject having a cancer (e.g., a hematologic cancer, such as AML).

In some embodiments, the hematologic cancer is MDS. In some embodiments, the MDS is high-risk MDS. In some embodiments, the hematologic cancer is MDS, and the subject also has secondary AML (e.g., AML derived from MDS or AML arising from prior chemotherapeutic treatments).

In some embodiments, the methods disclosed herein comprise preventive treatment. For example, administering a treatment to a subject that is likely to be afflicted by cancer in accordance with the methods described herein. In some embodiments, a subject is likely to be afflicted by cancer if the subject is characterized by one or more of a high risk for a cancer, a genetic predisposition to a cancer (e.g., genetic risk factors), a previous episode of a cancer (e.g., new cancers and/or recurrence), a family history of a cancer, exposure to a cancer-inducing agent (e.g., an environmental agent), and pharmacogenomic information (the effect of genotype on the pharmacokinetic, pharmacodynamic or efficacy profile of a therapeutic).

In some embodiments, a subject is likely to be afflicted by cancer if the subject is characterized by a high risk for a cancer. In some embodiments, a subject is likely to be afflicted by cancer if the subject is characterized by a genetic predisposition to a cancer. In some embodiments, a genetic predisposition to a cancer is a genetic clinical factor, as is known in the art. Such clinical factors may include, by way of example, HNPCC, MLH1, MSH2, MSH6, PMS1, PMS2 for at least colon, uterine, small bowel, stomach, urinary tract cancers. In some embodiments, a subject is likely to be afflicted by cancer if the subject is characterized by a previous episode of a cancer. In some embodiments, the subject has been afflicted with 1, 2, 3, 4, 5, or 6, previous episodes of cancer. In some embodiments, a subject is likely to be afflicted by cancer if the subject is characterized by a family history of a cancer. In some embodiments, a parent and/or grandparent and/or sibling and/or aunt/uncle and/or great aunt/great uncle, and/or cousin has been or is afflicted with a cancer. In some embodiments, a subject is likely to be afflicted by cancer if the subject is characterized by exposure to a cancer-inducing agent (e.g., an environmental agent). For example, exposing skin to strong sunlight is a clinical factor for skin cancer. By way of example, smoking is a clinical factor for cancers of the lung, mouth, larynx, bladder, kidney, and several other organs.

In embodiments, methods of the present disclosure include administering the therapeutic agents described herein to the subject based on mitochondrial integrity and/or MCL-1 dependency data obtained by contacting a subject's cancer cell with a profiling peptide.

Generally, profiling peptides comprise an Mcl-1 binding domain having a sequence shown in Table 1, which may be optionally modified.

TABLE 1
Exemplary Mcl-1 Binding Domains.

| SEQ ID NO: | Sequence |
|---|---|
| 1 | RPEIWMTQGLRRLGDEINAYYAR |
| 2 | RPEIWLTQSLQRLGDEINAYYAR |
| 3 | RPEIWLTQHLQRLGDEINAYYAR |
| 4 | RPEIWMGQGLRRLGDEINAYYAR |
| 5 | RPEIWLGQSLQRLGDEINAYYAR |
| 6 | RPEIWLGQHLQRLGDEINAYYAR |
| 7 | RPEIWITQELRRIGDEFNAYYAR |
| 8 | RPEIWMTQELRRIGDEFNAYYAR |
| 9 | RPEIWITQGLRRIGDEFNAYYAR |
| 10 | RPEIWITQELRRLGDEFNAYYAR |
| 11 | RPEIWITQELRRIGDEINAYYAR |

In some embodiments, a profiling peptide comprises an Mcl-1 binding domain having the sequence of any one of SEQ ID NOS:1-11 with 0-8 modifications. In some embodiments, a profiling peptide comprises an Mcl-1 binding domain having the sequence of any one of SEQ ID NOS:1-11 with 1-8 modifications.

“Modified” peptides include peptides having one or more amino acid substitutions as compared to a sequence disclosed herein. The substitution can be a conservative or a non-conservative substitution. Modified peptides also include peptides having additions of amino acids to, or deletions of amino acids from, the original peptide sequence. Therefore, modified peptides include fragments of the original peptide sequence. In some embodiments, each modification independently comprises a conservative amino acid substitution, an addition, or a deletion.

As used herein, a “modification” refers to a substitution, addition, or deletion of a single amino acid. Accordingly, when a number of modifications is referenced (e.g., an Mcl-1 binding domain having the sequence of SEQ ID NO:1 with two modifications), the number refers to the number of amino acids of the sequence that may be substituted, added, or deleted. In other words, each “substitution,” “addition,” or “deletion” replaces, adds, or removes a single amino acid, respectively, and does not refer to a single instance that replaces, adds, or removes more than one amino acid.

Modifications may be introduced by altering a polynucleotide encoding a profiling peptide, and may be performed by a variety of methods, including site-specific or site-directed mutagenesis. For example, mutations may be introduced at a particular location by synthesizing oligonucleotides containing a mutant sequence flanked by restriction sites enabling ligation to fragments of the unmodified sequence. Following ligation, the resulting sequence would encode a modified peptide having the desired amino acid addition, substitution, or deletion.

A “conservative substitution” includes a substitution found in one of the following conservative substitutions groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function or chemical structure or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Proline (Pro or P), and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

In embodiments, the modifications described herein may include the substitution of a naturally-occurring amino acid with a synthetic amino acid, amino acid analog, or amino acid mimetic, or the addition of a synthetic amino acid, amino acid analog, or amino acid mimetic. In such embodiments, modifications can include the substitution of one more L-amino acids with D-amino acids. The D-amino acid can be the same amino acid type as that found in the natural sequence or can be a different amino acid.

“Modification” also includes the substitution of a naturally-occurring amino acid with an amino acid that has been conjugated to, or otherwise associated with, a functional group. Such an amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. The presence of such amino acids may be preferred to, for example, increase polypeptide storage stability, and/or increase peptide solubility. Such modifications can be performed co-translationally or post-translationally during recombinant production, or by synthetic means.

In embodiments, the profiling peptides described herein comprise an Mcl-1 binding domain of any one of SEQ ID NOS:1-11 (e.g., SEQ ID NO:1) with 0 to 1 modifications. In some embodiments, the profiling peptides described herein comprise an Mcl-1 binding domain of any one of SEQ ID NOS:1-11 (e.g., SEQ ID NO:1) with 0 to 2 modifications. In some embodiments, the profiling peptides described herein comprise an Mcl-1 binding domain of any one of SEQ ID NOS:1-11 (e.g., SEQ ID NO:1) with 0 to 3 modifications. In some embodiments, the profiling peptides described herein comprise an Mcl-1 binding domain of any one of SEQ ID NOS:1-11 (e.g., SEQ ID NO:1) with 0 to 4 modifications. In some embodiments, the profiling peptides described herein comprise an Mcl-1 binding domain of any one of SEQ ID NOS:1-11 (e.g., SEQ ID NO:1) with 0 to 5 modifications. In some embodiments, the profiling peptides described herein comprise an Mcl-1 binding domain of any one of SEQ ID NOS:1-11 (e.g., SEQ ID NO:1) with 0 to 6 modifications. In some embodiments, the profiling peptides described herein comprise an Mcl-1 binding domain of any one of SEQ ID NOS:1-11 (e.g., SEQ ID NO:1) with 0 to 7 modifications. In some embodiments, the profiling peptides described herein comprise an Mcl-1 binding domain of any one of SEQ ID NOS:1-11 (e.g., SEQ ID NO:1) with 0 to 8 modifications. In some embodiments, the profiling peptides described herein comprise an Mcl-1 binding domain of any one of SEQ ID NOS:1-11 (e.g., SEQ ID NO:1) with 0 to 9 modifications, 0 to 10 modifications, 0 to 12 modifications, 0 to 15 modifications, or 0 to 20 modifications.

In some embodiments, the profiling peptides described herein comprise an Mcl-1 binding domain of any one of SEQ ID NOS:1-11 (e.g., SEQ ID NO:1) with 1 to 2 modifications. In some embodiments, the profiling peptides described herein comprise an Mcl-1 binding domain of any one of SEQ ID NOS:1-11 (e.g., SEQ ID NO:1) with 1 to 3 modifications. In some embodiments, the profiling peptides described herein comprise an Mcl-1 binding domain of any one of SEQ ID NOS:1-11 (e.g., SEQ ID NO:1) with 1 to 4 modifications. In some embodiments, the profiling peptides described herein comprise an Mcl-1 binding domain of any one of SEQ ID NOS:1-11 (e.g., SEQ ID NO:1) with 1 to 5 modifications. In some embodiments, the profiling peptides described herein comprise an Mcl-1 binding domain of any one of SEQ ID NOS:1-11 (e.g., SEQ ID NO:1) with 1 to 6 modifications. In some embodiments, the profiling peptides described herein comprise an Mcl-1 binding domain of any one of SEQ ID NOS:1-11 (e.g., SEQ ID NO:1) with 1 to 7 modifications. In some embodiments, the profiling peptides described herein comprise an Mcl-1 binding domain of any one of SEQ ID NOS:1-11 (e.g., SEQ ID NO:1) with 1 to 8 modifications. In some embodiments, the profiling peptides described herein comprise an Mcl-1 binding domain of any one of SEQ ID NOS:1-11 (e.g., SEQ ID NO:1) with 1 to 9 modifications. In some embodiments, the profiling peptides described herein comprise an Mcl-1 binding domain of any one of SEQ ID NOS:1-11 (e.g., SEQ ID NO:1) with 1 to 10 modifications, 1 to 12 modifications, 1 to 15 modifications, or 1 to 20 modifications.

In some embodiments, the profiling peptides described herein comprise an Mcl-1 binding domain of any one of SEQ ID NOS:1-11 (e.g., SEQ ID NO:1) with 2 to 3 modifications. In some embodiments, the profiling peptides described herein comprise an Mcl-1 binding domain of any one of SEQ ID NOS:1-11 (e.g., SEQ ID NO:1) with 2 to 4 modifications. In some embodiments, the profiling peptides described herein comprise an Mcl-1 binding domain of any one of SEQ ID NOS:1-11 (e.g., SEQ ID NO:1) with 2 to 5 modifications. In some embodiments, the profiling peptides described herein comprise an Mcl-1 binding domain of any one of SEQ ID NOS:1-11 (e.g., SEQ ID NO:1) with 2 to 6 modifications. In some embodiments, the profiling peptides described herein comprise an Mcl-1 binding domain of any one of SEQ ID NOS:1-11 (e.g., SEQ ID NO:1) with 2 to 7 modifications. In some embodiments, the profiling peptides described herein comprise an Mcl-1 binding domain of any one of SEQ ID NOS:1-11 (e.g., SEQ ID NO:1) with 2 to 8 modifications. In some embodiments, the profiling peptides described herein comprise an Mcl-1 binding domain of any one of SEQ ID NOS:1-11 (e.g., SEQ ID NO:1) with 2 to 9 modifications, 2 to 10 modifications, 2 to 12 modifications, 2 to 15 modifications, or 2 to 20 modifications.

In some embodiments, the profiling peptides described herein comprise an Mcl-1 binding domain of any one of SEQ ID NOS:1-11 (e.g., SEQ ID NO:1) with 3 to 4 modifications. In some embodiments, the profiling peptides described herein comprise an Mcl-1 binding domain of any one of SEQ ID NOS:1-11 (e.g., SEQ ID NO:1) with 3 to 5 modifications. In some embodiments, the profiling peptides described herein comprise an Mcl-1 binding domain of any one of SEQ ID NOS:1-11 (e.g., SEQ ID NO:1) with 3 to 6 modifications. In some embodiments, the profiling peptides described herein comprise an Mcl-1 binding domain of any one of SEQ ID NOS:1-11 (e.g., SEQ ID NO:1) with 3 to 7 modifications. In some embodiments, the profiling peptides described herein comprise an Mcl-1 binding domain of any one of SEQ ID NOS:1-11 (e.g., SEQ ID NO:1) with 3 to 8 modifications. In some embodiments, the profiling peptides described herein comprise an Mcl-1 binding domain of any one of SEQ ID NOS:1-11 (e.g., SEQ ID NO:1) with 3 to 9 modifications, 3 to 10 modifications, 3 to 12 modifications, 3 to 15 modifications, 3 to 20 modifications, 4 to 5 modifications, 4 to 6 modifications, 4 to 7 modifications, 4 to 8 modifications, 4 to 9 modifications, 4 to 10 modifications, 4 to 12 modifications, 4 to 15 modifications, 4 to 20 modifications, 5 to 6 modifications, 5 to 7 modifications, 5 to 8 modifications, 5 to 9 modifications, 5 to 10 modifications, 5 to 12 modifications, 5 to 15 modifications, 5 to 20 modifications, 6 to 7 modifications, 6 to 8 modifications, 6 to 9 modifications, 6 to 10 modifications, 7 to 8 modifications, 7 to 9 modifications, 7 to 10 modifications, 8 to 9 modifications, 8 to 10 modifications, or 9 to 10 modifications. In some embodiments, the profiling peptides described herein comprise a modified Mcl-1 binding domain of any one of SEQ ID NOS:1-11 (e.g., SEQ ID NO:1) with 1, 2, 3, 4, 5, 6, 7, 8, 9, 10, 11, 12, 13, 14, 15, 16, 17, 18, 19, or 20 modifications. The Mcl-1 binding domain sequence included in a profiling peptide of the present disclosure may include a modification at any position.

In embodiments where the Mcl-1 binding domain is a fragment of any one of SEQ ID NOS:1-11, the amino acid sequence can have a minimum length of 5 amino acids, 6 amino acids, 7 amino acids, 8 amino acids, 9 amino acids, 10 amino acids, 11 amino acids, 12 amino acids, 13 amino acids, or 14 amino acids. In some embodiments where the Mcl-1 binding domain is a fragment of any one of SEQ ID NOS:1-11, the amino acid sequence can have a minimum length of 15 amino acids. In some embodiments where the Mcl-1 binding domain is a fragment of any one of SEQ ID NOS:1-11, the amino acid sequence can have a minimum length of 16 amino acids. In some embodiments where the Mcl-1 binding domain is a fragment of any one of SEQ ID NOS:1-11, the amino acid sequence can have a minimum length of 17 amino acids. In some embodiments where the Mcl-1 binding domain is a fragment of any one of SEQ ID NOS:1-11, the amino acid sequence can have a minimum length of 18 amino acids. In some embodiments where the Mcl-1 binding domain is a fragment of any one of SEQ ID NOS:1-11, the amino acid sequence can have a minimum length of 19 amino acids. In some embodiments where the Mcl-1 binding domain is a fragment of any one of SEQ ID NOS:1-11, the amino acid sequence can have a minimum length of 20 amino acids. In some embodiments where the Mcl-1 binding domain is a fragment of any one of SEQ ID NOS:1-11, the amino acid sequence can have a minimum length of 21 amino acids.

In some embodiments, the Mcl-1 binding domain is a fragment of SEQ ID NO:1, and the amino acid sequence has a minimum length of 5 amino acids, 6 amino acids, 7 amino acids, 8 amino acids, 9 amino acids, 10 amino acids, 11 amino acids, 12 amino acids, 13 amino acids, or 14 amino acids. In some embodiments, the Mcl-1 binding domain is a fragment of SEQ ID NO:1, and the amino acid sequence has a minimum length of 15 amino acids. In some embodiments, the Mcl-1 binding domain is a fragment of SEQ ID NO:1, and the amino acid sequence has a minimum length of 16 amino acids. In some embodiments, the Mcl-1 binding domain is a fragment of SEQ ID NO:1, and the amino acid sequence has a minimum length of 17 amino acids. In some embodiments, the Mcl-1 binding domain is a fragment of SEQ ID NO:1, and the amino acid sequence has a minimum length of 18 amino acids. In some embodiments, the Mcl-1 binding domain is a fragment of SEQ ID NO:1, and the amino acid sequence has a minimum length of 19 amino acids. In some embodiments, the Mcl-1 binding domain is a fragment of SEQ ID NO:1, and the amino acid sequence has a minimum length of 20 amino acids. In some embodiments, the Mcl-1 binding domain is a fragment of SEQ ID NO:1, and the amino acid sequence has a minimum length of 21 amino acids.

In further embodiments, the Mcl-1 binding domain comprises at least 10 contiguous amino acids of any one of SEQ ID NOS:1-11 (e.g., SEQ ID NO:1). In some embodiments, the Mcl-1 binding domain comprises at least 11 contiguous amino acids of any one of SEQ ID NOS:1-11. In some embodiments, the Mcl-1 binding domain comprises at least 12 contiguous amino acids of any one of SEQ ID NOS:1-11. In some embodiments, the Mcl-1 binding domain comprises at least 13 contiguous amino acids of any one of SEQ ID NOS:1-11. In some embodiments, the Mcl-1 binding domain comprises at least 14 contiguous amino acids of any one of SEQ ID NOS:1-11. In some embodiments, the Mcl-1 binding domain comprises at least 15 contiguous amino acids of any one of SEQ ID NOS:1-11 (e.g., SEQ ID NO:1). In some embodiments, the Mcl-1 binding domain comprises at least 16 contiguous amino acids of any one of SEQ ID NOS:1-11. In some embodiments, the Mcl-1 binding domain comprises at least 17 contiguous amino acids of any one of SEQ ID NOS:1-11. In some embodiments, the Mcl-1 binding domain comprises at least 18 contiguous amino acids of any one of SEQ ID NOS:1-11. In some embodiments, the Mcl-1 binding domain comprises at least 19 contiguous amino acids of any one of SEQ ID NOS:1-11. In some embodiments, the Mcl-1 binding domain comprises at least 20 contiguous amino acids of any one of SEQ ID NOS:1-11 (e.g., SEQ ID NO:1). In some embodiments, the Mcl-1 binding domain comprises at least 21 contiguous amino acids of any one of SEQ ID NOS:1-11. In some embodiments, the Mcl-1 binding domain comprises at least at least 10 contiguous amino acids of SEQ ID NO:1, at least 11 contiguous amino acids of SEQ ID NO:1, at least 12 contiguous amino acids of SEQ ID NO:1, at least 13 contiguous amino acids of SEQ ID NO:1, at least 14 contiguous amino acids of SEQ ID NO:1, at least 15 contiguous amino acids of SEQ ID NO:1, at least 16 contiguous amino acids of SEQ ID NO:1, at least 17 contiguous amino acids of SEQ ID NO:1, at least 18 contiguous amino acids of SEQ ID NO:1, at least 19 contiguous amino acids of SEQ ID NO:1, at least 20 contiguous amino acids of SEQ ID NO:1, or at least 21 contiguous amino acids of SEQ ID NO:1.

In some embodiments, the Mcl-1 binding domain comprises no more than 10 contiguous amino acids of any one of SEQ ID NOS:1-11, no more than 11 contiguous amino acids of any one of SEQ ID NOS:1-11, no more than 12 contiguous amino acids of any one of SEQ ID NOS:1-11, no more than 13 contiguous amino acids of any one of SEQ ID NOS:1-11, no more than 14 contiguous amino acids of any one of SEQ ID NOS:1-11, no more than 15 contiguous amino acids of any one of SEQ ID NOS:1-11, no more than 16 contiguous amino acids of any one of SEQ ID NOS:1-11, no more than 17 contiguous amino acids of any one of SEQ ID NOS:1-11, no more than 18 contiguous amino acids of any one of SEQ ID NOS:1-11, no more than 19 contiguous amino acids of any one of SEQ ID NOS:1-11, no more than 20 contiguous amino acids of any one of SEQ ID NOS:1-11, or no more than 21 contiguous amino acids of any one of SEQ ID NOS:1-11. In some embodiments, the Mcl-1 binding domain comprises no more than 10 contiguous amino acids of SEQ ID NO:1, no more than 11 contiguous amino acids of SEQ ID NO:1, no more than 12 contiguous amino acids of SEQ ID NO:1, no more than 13 contiguous amino acids of SEQ ID NO:1, no more than 14 contiguous amino acids of SEQ ID NO:1, no more than 15 contiguous amino acids of SEQ ID NO:1, no more than 16 contiguous amino acids of SEQ ID NO:1, no more than 17 contiguous amino acids of SEQ ID NO:1, no more than 18 contiguous amino acids of SEQ ID NO:1, no more than 19 contiguous amino acids of SEQ ID NO:1, no more than 20 contiguous amino acids of SEQ ID NO:1, or no more than 21 contiguous amino acids of SEQ ID NO:1.

Embodiments of the Mcl-1 binding domains disclosed herein include amino acid sequences with at least 70% sequence identity to the sequence of any one of SEQ ID NOS:1-11. In some embodiments, the Mcl-1 binding domain has at least 75% sequence identity with the sequence of any one of SEQ ID NOS:1-11. In some embodiments, the Mcl-1 binding domain has at least 80% sequence identity with the sequence of any one of SEQ ID NOS:1-11. In some embodiments, the Mcl-1 binding domain has at least 85% sequence identity with the sequence of any one of SEQ ID NOS:1-11. In some embodiments, the Mcl-1 binding domain has at least 90% sequence identity with the sequence of any one of SEQ ID NOS:1-11. In some embodiments, the Mcl-1 binding domain has at least 95% sequence identity with the sequence of any one of SEQ ID NOS:1-11. In some embodiments, the Mcl-1 binding domain has at least 96% sequence identity with the sequence of any one of SEQ ID NOS:1-11. In some embodiments, the Mcl-1 binding domain has at least 97% sequence identity with the sequence of any one of SEQ ID NOS:1-11. In some embodiments, the Mcl-1 binding domain has at least 98% sequence identity with the sequence of any one of SEQ ID NOS:1-11. In some embodiments, the Mcl-1 binding domain has at least 99% sequence identity with the sequence of any one of SEQ ID NOS:1-11.

In some embodiments, the Mcl-1 binding domain has a sequence with at least 70% sequence identity to the sequence of SEQ ID NO:1. In some embodiments, the Mcl-1 binding domain has a sequence with at least 75% sequence identity to the sequence of SEQ ID NO:1. In some embodiments, the Mcl-1 binding domain has a sequence with at least 80% sequence identity to the sequence of SEQ ID NO:1. In some embodiments, the Mcl-1 binding domain has a sequence with at least 85% sequence identity to the sequence of SEQ ID NO:1. In some embodiments, the Mcl-1 binding domain has a sequence with at least 90% sequence identity to the sequence of SEQ ID NO:1. In some embodiments, the Mcl-1 binding domain has a sequence with at least 95% sequence identity to the sequence of SEQ ID NO:1. In some embodiments, the Mcl-1 binding domain has a sequence with at least 96% sequence identity to the sequence of SEQ ID NO:1. In some embodiments, the Mcl-1 binding domain has a sequence with at least 97% sequence identity to the sequence of SEQ ID NO:1. In some embodiments, the Mcl-1 binding domain has a sequence with at least 98% sequence identity to the sequence of SEQ ID NO:1. In some embodiments, the Mcl-1 binding domain has a sequence with at least 99% sequence identity to the sequence of SEQ ID NO:1.

“Percent sequence identity” refers to a relationship between two or more sequences, as determined by comparing the sequences. Preferred methods to determine sequence identity are designed to give the best match between the sequences tested. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. In embodiments, the length of a sequence aligned for comparison purposes is at least 70%, 80%, 90%, or 100% of the length of the reference sequence. In embodiments, the percent sequence identity referenced herein is calculated over the length of the reference sequence. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure it will be understood that where sequence analysis software is used for analysis, the results of the analysis are based on the “default values” of the program referenced. “Default values” mean any set of values or parameters which originally load with the software when first initialized.

In embodiments, a modified Mcl-1 binding domain retains the specificity and affinity for binding to Mcl-1 of the unmodified sequence (i.e., the modifications to the Mcl-1 binding domain do not alter the specificity or affinity for binding to Mcl-1 in a statistically significant, clinically significant, or biologically significant manner). In some embodiments, a modified Mcl-1 binding domain retains the specificity and affinity for binding to Mcl-1 of the unmodified sequence if the specificity and affinity of the modified Mcl-1 binding domain are at least 70%, 80%, 85%, 90%, 95%, 97%, or 99% of the specificity and affinity of the unmodified sequence. For example, a modified Mcl-1 binding domain may retain the specificity and affinity for binding to Mcl-1 of the unmodified sequence if the specificity and affinity of the modified Mcl-1 binding domain are at least at least 70%, 80%, 85%, 90%, 95%, 97%, or 99% of the specificity and affinity of any one of SEQ ID NOS:1-11.

In embodiments, the Mcl-1 binding domain binds to Mcl-1 with at least a 20-fold increased affinity over NOXA. In some embodiments, the Mcl-1 binding domain binds to Mcl-1 with at least a 22-fold increased affinity over NOXA. In some embodiments, the Mcl-1 binding domain binds to Mcl-1 with at least a 24-fold increased affinity over NOXA. In particular embodiments, the Mcl-1 binding domain binds to Mcl-1 with at least a 28-fold increased affinity over NOXA.

The effect of any amino acid modification to an Mcl-1 binding domain may be determined empirically by testing the resulting modified Mcl-1 binding domain for the ability to function in a biological assay, or to bind to a target molecule, such as a monoclonal or polyclonal antibody. For example, the ability of the modified Mcl-1 binding domain to fold into a conformation comparable to the unmodified sequence can be tested using assays known in the art, including reacting with monoclonal or polyclonal antibodies that are specific for the native or unfolded peptides, testing the retention of binding functions, and testing the sensitivity or resistance of the modified Mcl-1 binding domain to digestion with proteases.

Analysis and/or computer modeling of the primary and secondary amino acid structure of the Mcl-1 binding domain to analyze the tertiary structure of the peptide may aid in identifying specific amino acid residues that can be substituted, added, or deleted without significantly altering the structure and as a consequence, potentially significantly reducing the binding specificity and affinity of the Mcl-1 binding domain.

In embodiments, the Mcl-1 binding domain has a sequence of any one of SEQ ID NOS:1-11. In some embodiments, the Mcl-1 binding domain has a sequence of SEQ ID NO:1.

In embodiments, profiling peptides of the present disclosure further comprise a cellular uptake moiety, which is optionally joined to the Mcl-1 binding domain by a linker. A “cellular uptake moiety” refers to an amino acid sequence or chemical compound that, when conjugated to a peptide, allows the peptide and the cellular uptake moiety to cross the outer cell membrane, thereby transferring the peptide into the cell. Additionally, in some embodiments, the cellular uptake moiety may act as a targeting moiety, such that it directs the peptide to a desired cellular location (e.g., the mitochondria).

In embodiments, the cellular uptake moiety is a peptide sequence. In such embodiments, the cellular uptake moiety peptide is at least four amino acids in length, at least five amino acids in length, at least six amino acids in length, at least seven amino acids in length, at least eight amino acids in length, or at least nine amino acids in length. In some embodiments, the cellular uptake moiety comprises an amino acid sequence of 1 to 20 amino acids, 5 to 20 amino acids, 6 to 20 amino acids, 7 to 20 amino acids, 8 to 20 amino acids, 9 to 20 amino acids, 10 to 20 amino acids, 11 to 20 amino acids, 12 to 20 amino acids, 15 to 20 amino acids, 1 to 15 amino acids, 5 to 15 amino acids, 6 to 15 amino acids, 7 to 15 amino acids, 8 to 15 amino acids, 9 to 15 amino acids, 10 to 15 amino acids, 11 to 15 amino acids, 12 to 15 amino acids, 1 to 12 amino acids, 5 to 12 amino acids, 6 to 12 amino acids, 7 to 12 amino acids, 8 to 12 amino acids, 9 to 12 amino acids, 10 to 12 amino acids, 1 to 10 amino acids, 5 to 10 amino acids, 6 to 10 amino acids, or 7 to 10 amino acids.

In embodiments, the cellular uptake moiety peptide is a transduction domain isolated from a known peptide sequence. Peptides with transduction domains are well known in the art and include, for example, human immunodeficiency virus (HIV) Trans-Activator of Transcription (TAT; described in Vives et al. J Biol Chem. 1997 Jun. 20;272(25):16010-7), Herpes simplex virus tegument protein VP22, Atennapedia plasma membrane (ANT) translocation domain, a poly-Arg sequence, and the like. In embodiments, the cellular uptake moiety peptide is a continuous amino acid sequence from a known transduction domain. In other embodiments, the cellular uptake moiety peptide is two or more amino acid sequences from one or more known transduction domains that are not naturally present in a contiguous amino acid sequence, for example, a cellular uptake domain comprising two amino acid sequences would be separated by a third amino acid sequence in nature.

In embodiments, the cellular uptake moiety is an arginine rich amino acid sequence, such as a poly-Arg sequence. An amino acid sequence is “arginine rich” if greater than 50% of the amino acids of the cellular uptake moiety are arginine. In some embodiments, the arginine rich amino acid sequence includes 3 to 9 Arg residues, 3 to 10 Arg residues, 3 to 11 Arg residues, 3 to 12 Arg residues, 4 to 9 Arg residues, 4 to 10 Arg residues, 4 to 11 Arg residues, 4 to 12 Arg residues, 5 to 9 Arg residues, 5 to 10 Arg residues, 5 to 11 Arg residues, 5 to 12 Arg residues, 6 to 9 Arg residues, 6 to 10 Arg residues, 6 to 11 Arg residues, 6 to 12 Arg residues, 7 to 9 Arg residues, 7 to 10 Arg residues, 7 to 11 Arg residues, 7 to 12 Arg residues, 8 to 9 Arg residues, 8 to 10 Arg residues, 8 to 11 Arg residues, 8 to 12 Arg residues, 9 to 10 Arg residues, 9 to 11 Arg residues, or 9 to 12 Arg residues. In some embodiments, the poly-Arg sequence includes 3 Arg residues, 4 Arg residues, 5 Arg residues, 6 Arg residues, 7 Arg residues, 8 Arg residues, 9 Arg residues, 10 Arg residues, 11 Arg residues, or 12 Arg residues. In some embodiments, the poly-Arg sequence includes 2 to 10 contiguous Arg residues. In some embodiments, the poly-Arg sequence includes 3 to 9 contiguous Arg residues, 3 to 10 contiguous Arg residues, 3 to 11 contiguous Arg residues, 3 to 12 contiguous Arg residues, 4 to 9 contiguous Arg residues, 4 to 10 contiguous Arg residues, 4 to 11 contiguous Arg residues, 4 to 12 contiguous Arg residues, 5 to 9 contiguous Arg residues, 5 to 10 contiguous Arg residues, 5 to 11 contiguous Arg residues, 5 to 12 contiguous Arg residues, 6 to 9 contiguous Arg residues, 6 to 10 contiguous Arg residues, 6 to 11 contiguous Arg residues, 6 to 12 contiguous Arg residues, 7 to 9 contiguous Arg residues, 7 to 10 contiguous Arg residues, 7 to 11 contiguous Arg residues, 7 to 12 contiguous Arg residues, 8 to 9 contiguous Arg residues, 8 to 10 contiguous Arg residues, 8 to 11 contiguous Arg residues, 8 to 12 contiguous Arg residues, 9 to 10 contiguous Arg residues, 9 to 11 contiguous Arg residues, or 9 to 12 contiguous Arg residues.

In embodiments, the profiling peptide of the present disclosure comprises an arginine rich amino sequence and an Mcl-1 binding domain having a sequence of any one of SEQ ID NOS:1-11 with 0-8 modifications. In some embodiments, the profiling peptide of the present disclosure comprises an arginine rich amino sequence and an Mcl-1 binding domain having a sequence of any one of SEQ ID NOS:1-11 with 1-8 modifications. In embodiments, the profiling peptide of the present disclosure comprises an arginine rich amino acid sequence and an Mcl-1 binding domain having the sequence of SEQ ID NO:1 with 0-8 modifications. In some embodiments, the profiling peptide of the present disclosure comprises a poly-Arg sequence having 2 to 10 contiguous Arg residues and an Mcl-1 binding domain having a sequence of any one of SEQ ID NOS:1-11 with 0-8 modifications. In some embodiments, the profiling peptide of the present disclosure comprises a poly-Arg sequence having 2 to 10 contiguous Arg residues and an Mcl-1 binding domain having a sequence of any one of SEQ ID NOS:1-11 with 1-8 modifications.

In embodiments, the profiling peptide of the present disclosure comprises an arginine rich amino sequence and an Mcl-1 binding domain having a sequence of any one of SEQ ID NOS:1-11. In some embodiments, the profiling peptide of the present disclosure comprises a poly-Arg sequence having 3 to 10 contiguous Arg residues and an Mcl-1 binding domain having a sequence of any one of SEQ ID NOS:1-11 with 0-8 modifications. In some embodiments, the profiling peptide of the present disclosure comprises a poly-Arg sequence having 3 to 10 contiguous Arg residues and an Mcl-1 binding domain having a sequence of any one of SEQ ID NOS:1-11 with 1-8 modifications. In embodiments, the profiling peptide of the present disclosure comprises a poly-Arg sequence having 3 to 10 Arg residues and an Mcl-1 binding domain having the sequence of SEQ ID NO:1 with 0-8 modifications. In some embodiments, the profiling peptide of the present disclosure comprises a poly-Arg sequence having 3 to 10 contiguous Arg residues and an Mcl-1 binding domain having a sequence of any one of SEQ ID NOS:1-11.

In embodiments, the cellular uptake moiety peptide is an optionally modified transduction domain from a known peptide. The modifications may be made using known techniques.

In embodiments, the cellular uptake moiety peptide is an optionally modified TAT translocation domain. The optionally modified TAT translocation domain can have 0 to 1 modifications, 0 to 2 modifications, 0 to 3 modifications, 0 to 4 modifications, 0 to 5 modifications, 0 to 6 modifications, 0 to 7 modifications, 0 to 8 modifications, 0 to 9 modifications, 1 to 2 modifications, 1 to 3 modifications, 1 to 4 modifications, 1 to 5 modifications, 1 to 6 modifications, 1 to 7 modifications, 1 to 8 modifications, 1 to 9 modifications, 2 to 3 modifications, 2 to 4 modifications, 2 to 5 modifications, 2 to 6 modifications, 2 to 7 modifications, 2 to 8 modifications, 2 to 9 modifications, 3 to 4 modifications, 3 to 5 modifications, 3 to 6 modifications, 3 to 7 modifications, 3 to 8 modifications, 3 to 9 modifications, 4 to 5 modifications, 4 to 6 modifications, 4 to 7 modifications, 4 to 8 modifications, or 4 to 9 modifications. In embodiments where the cellular uptake moiety peptide is a fragment of the TAT translocation domain, the cellular uptake moiety peptide sequence can have a minimum length of 5 amino acids, 6 amino acids, 7 amino acids, 8 amino acids, 9 amino acids, or 10 amino acids. In some embodiments where the cellular uptake moiety peptide is a fragment of the TAT translocation domain, the cellular uptake moiety peptide sequence can have a minimum of 5 contiguous amino acids of a TAT translocation domain. In some embodiments, the cellular uptake moiety peptide sequence can have a minimum of 5 contiguous amino acids, 6 contiguous amino acids, 7 contiguous amino acids, 8 contiguous amino acids, 9 contiguous amino acids, or 10 contiguous amino acids of a TAT translocation domain. Modified TAT translocation domains disclosed herein include amino acid sequences with at least 70% sequence identity, at least 75% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to the sequence of YGRKKRRQRRR (SEQ ID NO:12). In some embodiments, the cellular uptake moiety has at least 90% identity with the sequence of SEQ ID NO:12. In some embodiments, the cellular uptake moiety is a modified TAT translocation domain. In other embodiments, the cellular uptake moiety is a TAT translocation domain comprising the sequence of SEQ ID NO:12.

In embodiments, the profiling peptide of the present disclosure comprises a TAT translocation domain and an Mcl-1 binding domain having a sequence of SEQ ID NOS:1-11 with 0-8 modifications. In some embodiments, the profiling peptide of the present disclosure comprises a TAT translocation domain and an Mcl-1 binding domain having a sequence of SEQ ID NOS:1-11 with 1-8 modifications. In embodiments, the profiling peptide of the present disclosure comprises a TAT translocation domain and an Mcl-1 binding domain having the sequence of SEQ ID NO:1 with 0-8 modifications. In some embodiments, the profiling peptide of the present disclosure comprises a TAT translocation domain and an Mcl-1 binding domain having a sequence of any one of SEQ ID NOS:1-11. In some embodiments, the profiling peptide of the present disclosure comprises a TAT translocation domain having the sequence of SEQ ID NO:12 and an Mcl-1 binding domain having a sequence of any one of SEQ ID NOS:1-11 with 0-8 modifications. In some embodiments, the profiling peptide of the present disclosure comprises a TAT translocation domain having the sequence of SEQ ID NO:12 and an Mcl-1 binding domain having a sequence of any one of SEQ ID NOS:1-11 with 1-8 modifications. In embodiments, the profiling peptide of the present disclosure comprises a TAT translocation domain having the sequence of SEQ ID NO:12 and an Mcl-1 binding domain having the sequence of SEQ ID NO:1 with 0-8 modifications. In some embodiments, the profiling peptide of the present disclosure comprises a TAT translocation domain having the sequence of SEQ ID NO:12 and an Mcl-1 binding domain having a sequence of any one of SEQ ID NOS:1-11.

In embodiments, the cellular uptake moiety peptide is an optionally modified ANT translocation domain. The optionally modified ANT translocation domain can have 0 to 1 modifications, 0 to 2 modifications, 0 to 3 modifications, 0 to 4 modifications, 0 to 5 modifications, 0 to 6 modifications, 0 to 7 modifications, 0 to 8 modifications, 0 to 9 modifications, 1 to 2 modifications, 1 to 3 modifications, 1 to 4 modifications, 1 to 5 modifications, 1 to 6 modifications, 1 to 7 modifications, 1 to 8 modifications, 1 to 9 modifications, 2 to 3 modifications, 2 to 4 modifications, 2 to 5 modifications, 2 to 6 modifications, 2 to 7 modifications, 2 to 8 modifications, 2 to 9 modifications, 3 to 4 modifications, 3 to 5 modifications, 3 to 6 modifications, 3 to 7 modifications, 3 to 8 modifications, 3 to 9 modifications, 4 to 5 modifications, 4 to 6 modifications, 4 to 7 modifications, 4 to 8 modifications, or 4 to 9 modifications. In embodiments where the cellular uptake moiety peptide is a fragment of the ANT translocation domain, the cellular uptake moiety peptide sequence can have a minimum length of 5 amino acids, 6 amino acids, 7 amino acids, 8 amino acids, 9 amino acids, or 10 amino acids. In some embodiments where the cellular uptake moiety peptide is a fragment of the ANT translocation domain, the cellular uptake moiety peptide sequence can have a minimum of 5 contiguous amino acids of an ANT translocation domain. In some embodiments, the cellular uptake moiety peptide sequence can have a minimum of 6 contiguous amino acids, 7 contiguous amino acids, 8 contiguous amino acids, 9 contiguous amino acids, or 10 contiguous amino acids of an ANT translocation domain. Modified ANT translocation domains disclosed herein include amino acid sequences with at least 70% sequence identity, at least 75% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to the sequence of RQIKIWFQNRRMKWKK (SEQ ID NO:13). In some embodiments, the cellular uptake moiety has at least 90% identity with the sequence of SEQ ID NO:13. In some embodiments, the cellular uptake moiety peptide is a modified ANT translocation domain. In other embodiments, the cellular uptake moiety peptide comprises an ANT translocation domain having the sequence of SEQ ID NO:13.

In embodiments, the profiling peptide of the present disclosure comprises an ANT translocation domain and an Mcl-1 binding domain having a sequence of any one of SEQ ID NOS:1-11 with 0-8 modifications. In some embodiments, the profiling peptide of the present disclosure comprises an ANT translocation domain and an Mcl-1 binding domain having a sequence of any one of SEQ ID NOS:1-11 with 1-8 modifications. In embodiments, the profiling peptide of the present disclosure comprises an ANT translocation domain and an Mcl-1 binding domain having the sequence of SEQ ID NO:1 with 0-8 modifications. In embodiments, the profiling peptide of the present disclosure comprises an ANT translocation domain and an Mcl-1 binding domain having a sequence of any one of SEQ ID NOS:1-11. In some embodiments, the profiling peptide of the present disclosure comprises an ANT translocation domain having the sequence of SEQ ID NO:13 and an Mcl-1 binding domain having a sequence of any one of SEQ ID NOS:1-11 with 0-8 modifications. In some embodiments, the profiling peptide of the present disclosure comprises an ANT translocation domain having the sequence of SEQ ID NO:13 and an Mcl-1 binding domain having a sequence of any one of SEQ ID NOS:1-11 with 1-8 modifications. In embodiments, the profiling peptide of the present disclosure comprises an ANT translocation domain having the sequence of SEQ ID NO:13 and an Mcl-1 binding domain having the sequence of SEQ ID NO:1 with 0-8 modifications. In some embodiments, the profiling peptide of the present disclosure comprises an ANT translocation domain having the sequence of SEQ ID NO:13 and an Mcl-1 binding domain having a sequence of any one of SEQ ID NOS:1-11.

In embodiments, a modified cellular uptake moiety peptide retains the ability of the unmodified sequence to cross the cell membrane when conjugated to a peptide (i.e., the modifications to the cellular uptake moiety peptide do not alter the ability to cross the cell membrane when conjugated to a peptide in a statistically significant, clinically significant, or biologically significant manner). In some embodiments, a modified cellular uptake moiety peptide retains the ability of the unmodified sequence to cross the cell membrane when conjugated to a peptide if the internalization efficiency of the modified cellular uptake moiety peptide is at least 70%, 80%, 85%, 90%, 95%, 97%, or 99% of the internalization efficiency of the unmodified sequence.

Alternatively, the cellular uptake moiety can be a chemical compound. Chemical compounds that facilitate cellular internalization are understood by one of skill in the art, and include, for example, cholesterol moieties, octanoic acid, lithocholic acid, oleyl alcohol, lithocholic acid oleylamide, and decanoic acid.

The Mcl-1 binding domain and the cellular uptake moiety can be linked by chemical coupling in any suitable manner known in the art. The cellular uptake moiety may be linked to the Mcl-1 binding domain at any suitable location. In some embodiments, the cellular uptake moiety is conjugated to the N-terminus of the Mcl-1 binding domain. In other embodiments, the cellular uptake moiety is conjugated to the C-terminus of the Mcl-1 binding domain.

In embodiments, the cellular uptake moiety is conjugated to the Mcl-1 binding domain via a linker. In further embodiments, the cellular uptake moiety is conjugated via a linker to the N-terminus of the Mcl-1 binding domain. In still further embodiments, the cellular uptake moiety is conjugated via a linker to the C-terminus of the Mcl-1 binding domain.

Suitable linkers include peptide sequences of any length and other chemical linkers as would be understood by one of ordinary skill. Short peptide sequences are employed in certain embodiments, for example peptide sequences including uncharged amino acids, non-polar amino acids and/or small amino acids. In some embodiments, a linker is an amino acid sequence of 1-5 amino acids. For example, some exemplary linkers include Gly, Pro, Ala, Val, Leu, Met, Ile, and/or Phe amino acids. Other examples of suitable peptide sequences include two Pro residues, three Gly residues, and the like. In some embodiments, the cellular uptake moiety is linked to the Mcl-1 binding domain in such a way that the cellular uptake moiety is cleaved upon or after entry into the cell. In certain embodiments, the linker comprises three Gly residues, for example GGG.

Embodiments of the profiling peptides of the present disclosure may be 20 to 40 amino acids in length, 20 to 45 amino acids in length, 20 to 50 amino acids in length, 25 to 40 amino acids in length, 25 to 45 amino acids in length, 25 to 50 amino acids in length, 30 to 36 amino acids in length, 30 to 37 amino acids in length, 30 to 38 amino acids in length, 30 to 39 amino acids in length, 30 to 40 amino acids in length, 30 to 45 amino acids in length, 30 to 50 amino acids in length, 31 to 36 amino acids in length, 31 to 37 amino acids in length, 31 to 38 amino acids in length, 31 to 39 amino acids in length, 31 to 40 amino acids in length, 32 to 36 amino acids in length, 32 to 37 amino acids in length, 32 to 38 amino acids in length, 32 to 39 amino acids in length, 32 to 40 amino acids in length, 33 to 36 amino acids in length, 33 to 37 amino acids in length, 33 to 38 amino acids in length, 33 to 39 amino acids in length, 33 to 40 amino acids in length, 34 to 36 amino acids in length, 34 to 37 amino acids in length, 34 to 38 amino acids in length, 34 to 39 amino acids in length, 34 to 40 amino acids in length, 35 to 36 amino acids in length, 35 to 37 amino acids in length, 35 to 38 amino acids in length, 35 to 39 amino acids in length, 35 to 40 amino acids in length, 35 to 45 amino acids in length, 35 to 50 amino acids in length, 36 to 37 amino acids in length, 36 to 38 amino acids in length, 36 to 39 amino acids in length, 36 to 40 amino acids in length, 37 to 38 amino acids in length, 37 to 39 amino acids in length, 37 to 40 amino acids in length, 38 to 39 amino acids in length, 38 to 40 amino acids in length, or 39 to 40 amino acids in length.

In embodiments, a profiling peptide comprises a cellular uptake moiety, and an Mcl-1 binding domain having a sequence of any one of SEQ ID NOS:1-11 with 0-8 modifications. In embodiments, a profiling peptide comprises a cellular uptake moiety, and an Mcl-1 binding domain having a sequence of any one of SEQ ID NOS:1-11 with 1-8 modifications. In embodiments, a profiling peptide comprises a cellular uptake moiety, and an Mcl-1 binding domain having the sequence of SEQ ID NO:1 with 0-8 modifications. In embodiments, a profiling peptide comprises a cellular uptake moiety, and an Mcl-1 binding domain having a sequence of any one of SEQ ID NOS:1-11. In some embodiments, a profiling peptide comprises a cellular uptake moiety, and an Mcl-1 binding domain having the sequence of SEQ ID NO:1.

In embodiments, a profiling peptide comprises a cellular uptake moiety having the sequence of SEQ ID NO:12 conjugated to an Mcl-1 binding domain having a sequence of any one of SEQ ID NOS:1-11 with 0-8 modifications by a linker. In embodiments, a profiling peptide comprises a cellular uptake moiety having the sequence of SEQ ID NO:12 conjugated to an Mcl-1 binding domain having a sequence of any one of SEQ ID NOS:1-11 with 1-8 modifications by a linker. In embodiments, a profiling peptide comprises a cellular uptake moiety having the sequence of SEQ ID NO:12 conjugated to an Mcl-1 binding domain having the sequence of SEQ ID NO:1 with 0-8 modifications by a linker. In embodiments, a profiling peptide comprises a cellular uptake moiety having the sequence of SEQ ID NO:12 conjugated to an Mcl-1 binding domain having a sequence of any one of SEQ ID NOS:1-11 by a linker.

In embodiments, the profiling peptide has the sequence of

(SEQ ID NO: 14)
YGRKKRRQRRRGGGRPEIWMTQGLRRLGDEINAYYAR
or
(SEQ ID NO: 15)
RPEIWMTQGLRRLGDEINAYYARGGGYGRKKRRQRRR.

In certain embodiments, the profiling peptide comprises the sequence of SEQ ID NO:14. In other embodiments, the profiling peptide comprises the sequence of SEQ ID NO:15. In specific embodiments, the profiling peptide consists of the sequence of SEQ ID NO:14. In other embodiments, the profiling peptide consists of the sequence of SEQ ID NO:15.

Modified profiling peptides may be synthesized and purified by standard chemical methods. Peptides may be chemically synthesized by manual techniques or by automated procedures. Equipment for automated synthesis of peptides is commercially available from suppliers such as Perkin-Elmer, Inc. (Waltham, Mass.) and may be operated according to the manufacturer's instructions. Additionally, synthesized profiling peptides may be obtained from any number of different custom peptide synthesizing manufacturers. If required, synthesized peptides may be purified using preparative reverse phase chromatography, partition chromatography, gel filtration, gel electrophoresis, ion-exchange chromatography, or other methods used in the art.

Alternatively, modified profiling peptides may be readily prepared by genetic engineering and recombinant molecular biology methods and techniques. For example, polynucleotides encoding modified profiling peptides, or fragments thereof, may be constructed by recombinant methods or chemically synthesized (using such devices as an automatic synthesizer). Methods for purifying polynucleotides after either chemical synthesis or recombinant synthesis are known to persons skilled in the art. The constructed or synthesized polynucleotides may be incorporated into expression vectors (e.g., a plasmid, a viral particle, or a phage) for production of the profiling peptide in a host cell into which the expression vector has been introduced. Polynucleotides that encode a profiling peptide described herein may be recombinantly expressed in a variety of different host cells. Host cells may then be genetically engineered (transduced, transformed, or transfected) with the expression vectors. Selection and maintenance of culture conditions for particular host cells, such as temperature, pH and the like, will be readily apparent to the ordinarily skilled artisan. The produced peptides may then be harvested and purified using methods known in the art.

Such peptides may be used to produce a sensitivity profile for a cancer cell or a plurality of cancer cells. In certain embodiments, the cancer cell or plurality of cancer cells is from a human tumor-derived cell line. In certain embodiments, the cancer cell or plurality of cancer cells is cancer stem cells. In some embodiments, the cancer cell or plurality of cancer cells is isolated from a tumor. In certain embodiments, the cancer cell or plurality of cancer cells is derived from the biopsy of a non-solid tumor. In embodiments, the cancer cell or plurality of cancer cells is obtained from peripheral blood from the subject. In other embodiments, the cancer cell or plurality of cancer cells is obtained from bone marrow of the subject.

In embodiments, the cancer cells are from a solid cancer. In some embodiments, the cancer cell or plurality of cancer cells is derived from a solid tumor. In embodiments, the cancer cell or plurality of cancer cells is derived from the biopsy of a solid tumor, such as, for example, a biopsy of a colorectal, breast, prostate, lung, pancreatic, renal, or ovarian primary tumor. In some embodiments, the cancer cell or plurality of cancer cells is a circulating tumor cell. In embodiments, the cancer cells are from a non-solid cancer. In various embodiments, the cancer cells are from a pre-metastatic cancer. In various embodiments, the cancer cells are from a metastatic cancer.

In some embodiments, the cancer cell or plurality of cancer cells is derived from a subject with a hematologic cancer. In some embodiments, the cancer cell or plurality of cancer cells is derived from multiple myeloma, MDS, AML, ALL, acute lymphocytic leukemia, chronic lymphogenous leukemia, CLL, mantle cell lymphoma, diffuse large B-cell lymphoma, follicular lymphoma, or non-Hodgkin's lymphoma. In specific embodiments, the cancer cell or plurality of cancer cells is derived from the biopsy of a subject with multiple myeloma, AML, acute lymphocytic leukemia, chronic lymphogenous leukemia, mantle cell lymphoma, diffuse large B-cell lymphoma, and non-Hodgkin's lymphoma. In certain embodiments, the cancer is AML.

In a specific embodiment, the cancer cell or plurality of cancer cells is a multiple myeloma cell that is enriched by selection from a biopsy sample with an anti-CD138 antibody bound to a solid matrix or bead. In a specific embodiment, the cancer cell or plurality of cancer cells is an AML cell that is enriched by binding to a CD45-directed antibody. In a specific embodiment, the cancer cell or plurality of cancer cells is from a chronic lymphogenous leukemia or diffuse large B-cell lymphoma that is enriched by non-B cell depletion.

In various embodiments, the plurality of cancer cells is from a sample that has been frozen. In such embodiments, the sample may be warmed using a quick thaw process. The sample may then be added to culture medium and incubated.

In other embodiments, the plurality of cancer cells is from a sample that has not been frozen, i.e., that has been freshly collected. In such embodiments, the sample is added to culture medium and incubated after being isolated.

In some embodiments, such methods include isolating a cancer cell or a plurality of cancer cells from a subject. In embodiments, a plurality of cancer cells isolated from a subject is separated into two or more portions. In embodiments, a plurality of cancer cells isolated from a subject is separated into three or more portions. In some embodiments, multiple replicates are tested for each portion.

In embodiments, the cancer cell, the plurality of cancer cells, or a portion thereof, is contacted with one or more profiling peptides disclosed herein and a percent polarization is determined. In embodiments, the cancer cell, the plurality of cancer cells, or a portion thereof, is contacted with one or more profiling peptides disclosed herein and a change in mitochondrial integrity of the cell(s) is detected. In various embodiments, more than one profiling peptide may be used at once. In such embodiments, a panel of profiling peptides (e.g., 2, 3, 4, 5, 10, etc. profiling peptides) may be screened on a single subject specimen.

In some embodiments, the cancer cell, the plurality of cancer cells, or a portion thereof, is contacted with a composition comprising a profiling peptide. In such embodiments, the composition may comprise a profiling peptide in a concentration ranging from about 1.5 μM to about 2.5 μM. In embodiments, the composition comprises a profiling peptide in a concentration ranging from about 1.75 μM to about 2.25 μM. In embodiments, the composition comprises a profiling peptide in a concentration of about 2.0 μM.

In embodiments, the plurality of cancer cells, or a portion thereof, is contacted with one or more profiling peptides for about 15 minutes to about 45 minutes. In some embodiments, the plurality of cancer cells, or a portion thereof, is contacted with one or more profiling peptides for about 20 minutes to about 40 minutes. In some embodiments, the plurality of cancer cells, or a portion thereof, is contacted with one or more profiling peptides for about 30 minutes.

A percent polarization can be related to a change in mitochondrial integrity in the cell or plurality of cells. A change in mitochondrial integrity can be detected in any suitable manner, such as, for example, a change in mitochondrial membrane potential, chromatin condensation, loss of viability, Cytochrome C translocation from the mitochondrial intermembrane space to the cytosol, swelling of the mitochondria, mitochondrial fission, morphological changes (e.g., cell shrinkage, membrane blebbing, etc.), phosphatidyl serine externalization (e.g., as measured by annexin V staining) or the increase in reactive oxygen intermediates. As is understood by one of skill in the art, various methods of detection for each of the indications of a change in mitochondrial integrity may be employed. In embodiments, the change in mitochondrial integrity will be a decrease in mitochondrial integrity. In some embodiments, the decrease in mitochondrial integrity is measured by a decrease in mitochondrial membrane potential. The decrease in mitochondrial potential may be determined using any suitable method known in the art. In some embodiments, the decrease in mitochondrial integrity is measured by Cytochrome C leakage. In some embodiments, the decrease will be a statistically significant, clinically significant, or biologically significant decrease. In some embodiments, the decrease is a 2%, 5%, 10%, 15%, 20%, 25%, 30%, 35%, 40%, 45%, 50%, 60%, 70%, 80%, or 90% difference in a measurement of mitochondrial integrity, as described herein, as compared to a control.

A change in mitochondrial membrane potential may be measured using any suitable detecting agent. The detecting agent can be any suitable agent, such as a fluorescent dye, a non-fluorescent dye that can be converted to a fluorescent dye, an antibody, and the like. Fluorescent dyes include, for example, 5,5′,6,6′-Tetrachloro-1,1′,3,3′-tetraethyl-imidacarbocyanine iodide (JC-1), propidium iodide (PI), 1,1′,3,3,3′,3′-hexamethylindodicarbo-cyanine iodide (DilC1), and 3,3′-Dihexyloxacarbocyanine Iodide (DiOC6). In various embodiments, the fluorescent dye is a potentiometric dye. Suitable potentiometric dyes include, for example, DilC1, JC-1, and rhodamine 123. In embodiments, the potentiometric dye included is JC-1 or rhodamine 123. In other embodiments, the dye is dihydrorhodamine 123, a non-fluorescent dye that can be converted via oxidation to rhodamine 123, a fluorescent dye.

In embodiments, the plurality of cancer cells, or a portion thereof, is contacted with a dye in a concentration ranging from about 0.5 nM to about 1.5 nM. In embodiments, the plurality of cancer cells, or a portion thereof, is contacted with a dye in a concentration ranging from about 0.75 nM to about 1.25 nM. In embodiments, the plurality of cancer cells, or a portion thereof, is contacted with a dye in a concentration of about 1.0 μM. In embodiments, the plurality of cancer cells, or a portion thereof, is contacted with a dye for about 60 minutes to about 120 minutes. In embodiments, the plurality of cancer cells, or a portion thereof, is contacted with a dye for about 75 minutes to about 105 minutes. In embodiments, the plurality of cancer cells, or a portion thereof, is contacted with a dye for about 80 minutes to about 100 minutes. In embodiments, the plurality of cancer cells, or a portion thereof, is contacted with a dye for about 90 minutes.

In another example, Cytochrome C translocation can be measured using immunofluorescence staining. In a further example, an increase in reactive oxygen intermediates can be measured by flow cytometric analysis after staining with carboxy-dichlorofluorescin diacetate.

In various embodiments, the plurality of cancer cells is divided into two or more portions for the purposes of profiling. In embodiments, one portion is treated with a negative control and one portion is contacted with one or more profiling peptides or a composition comprising one or more profiling peptides disclosed herein. Any suitable negative control may be used. Examples of negative controls include water and water soluble organic solvents, such as DMSO, ethanol, and methanol. In some embodiments, the negative control is water.

Accordingly, methods of the present disclosure comprise contacting a first portion of a plurality of cancer cells with a profiling peptide comprising a cellular uptake moiety and an Mcl-1 binding domain having the sequence of SEQ ID NO:1 with 0-8 modifications; contacting a second portion of the plurality of cancer cells with a negative control; and determining a percent polarization of the first portion and the second portion of the plurality of cancer cells.

In some embodiments, one portion of the plurality of cancer cells is contacted with a positive control. In such embodiments, methods of the disclosure further comprise contacting a third portion of the plurality of cancer cells with a positive control. Any suitable positive control may be used. Examples of positive controls include Carbonyl cyanide-4-(trifluoromethoxy)phenylhydrazone (FCCP), Carbonyl cyanide m-chlorophenyl hydrazone (CCCP), N5,N6-bis(2-fluorophenyl)-[1,2,5]oxadiazolo[3,4-b]pyrazine-5,6-diamine (BAM-15), and the like. In particular embodiments, the positive control used is CCCP.

In embodiments, the plurality of cancer cells, or a portion thereof, is contacted with a positive control in a concentration ranging from about 25 μM to about 250 μM. In embodiments, the plurality of cancer cells, or a portion thereof, is contacted with a positive control in a concentration ranging from about 25 μM to about 200 μM. In embodiments, the plurality of cancer cells, or a portion thereof, is contacted with a positive control in a concentration ranging from about 50 μM to about 150 μM. In embodiments, the plurality of cancer cells, or a portion thereof, is contacted with a positive control in a concentration of about 100 μM.

In embodiments, at least a portion of the plurality of cancer cells is contacted with a blocking buffer that blocks receptors on the cells that bind to the Fragment crystallizable (FC) region of antibodies (i.e., FC receptors). In embodiments, the plurality of cancer cells is contacted with the blocking buffer before being separated into portions. In embodiments, the blocking buffer is incubated with the plurality of cancer cells at room temperature.

In some embodiments, a plurality of cancer cells is contacted with one or more labels. In embodiments, the plurality of cancer cells is contacted with a label before being separated into portions. In some embodiments that use flow cytometry, the labels are fluorophores attached to antibodies or a chemical entity with affinity for a cell membrane feature or other cellular structure. In other embodiments that use flow cytometry, the labels are quantum dots attached to antibodies or a chemical entity with affinity for a cell membrane feature or other cellular structure. In any of these embodiments, the antibodies or chemical entities may recognize any suitable cell surface marker, such as CD3, CD13, CD20, CD33, CD34, or CD45. In various embodiments, a combination of labels is used.

In embodiments, the label comprises at least one monoclonal antibody. In some embodiments, the at least one monoclonal antibody comprises an anti-CD45 antibody, an anti-CD3 antibody, an anti-CD20 antibody, an anti-CD13 antibody, an anti-CD33 antibody, an anti-CD34 antibody, an anti-CD117 antibody, anti-HLA-DR antibody, or a combination thereof. In specific embodiments, the at least one monoclonal antibody comprises an anti-CD45 antibody, an anti-CD3 antibody, an anti-CD20 antibody, an anti-CD13 antibody, an anti-CD33 antibody, an anti-CD34 antibody, or a combination thereof.

In some embodiments, an additive with a high affinity for calcium channels is added to the plurality of cancer cells, or a portion thereof. In some such embodiments, the additive is a diterpenoid. In particular embodiments, the additive is ryanodine. In embodiments, the additive is added in a concentration that is sufficient to significantly reduce or prevent nonspecific dye uptake. In some embodiments, the additive is added in a concentration of at least about 20 μM. In some embodiments, the additive is added in a concentration of at least about 30 μM. In embodiments, the diterpenoid is added in a concentration ranging from about 10 μM to about 50 μM. In embodiments, the diterpenoid is added in a concentration ranging from about 20 μM to about 40 μM. In embodiments, the diterpenoid is added in a concentration of about 30 μM.

In some embodiments, an ATP synthase inhibitor is added to the plurality of cancer cells, or a portion thereof. In some such embodiments, the ATP synthase inhibitor is an oligomycin. In particular embodiments, the oligomycin is oligomycin A. In some embodiments, the ATP synthase inhibitor is added in a concentration of at least 0.25 μm. In some embodiments, the ATP synthase inhibitor is added in a concentration of at least about 0.5 μm. In some embodiments, the ATP synthase inhibitor is added in a concentration of no more than about 1.0 μm. In embodiments, the ATP synthase inhibitor is added in a concentration ranging from about 0.25 μM to about 0.75 μM. In embodiments, the ATP synthase inhibitor is added in a concentration ranging from about 0.4 μM to about 0.6 μM. In embodiments, the ATP synthase inhibitor is added in a concentration of about 0.5 μM.

In embodiments, the plurality of cancer cells is then contacted with a detecting agent, as described above. In some embodiments, the detecting agent is a dye. In some embodiments, the detecting agent is a fluorescent dye. In some embodiments, the detecting agent is a potentiometric dye. In certain embodiments, the dye is JC-1, DiOC₆, or rhodamine 123. In certain embodiments, the dye is JC-1 or rhodamine 123. In particular embodiments, the dye is DiOC₆.

In some embodiments, the plurality of cancer cells, or a portion thereof, is washed prior to being contacted with the detecting agent.

In such embodiments, the plurality of cancer cells may then be analyzed using flow cytometry. In embodiments, determining the percent polarization of the first portion and the second portion of the plurality of cancer cells comprises analyzing the first portion and the second portion of the plurality of cancer cells by flow cytometry. In some embodiments, the third portion of the plurality of cancer cells is also analyzed by flow cytometry.

Any suitable gating may be used in flow cytometry analysis. In embodiments, analyzing the first portion, second portion, and third portion of the plurality of cancer cells by flow cytometry comprises gating on the positive control. In some embodiments, such gating is on the CD45 dim, CD13, CD33, and CD34 high population of blast cells. In other embodiments, such gating is the CD34 dim, CD3 and CD20 high population. Accordingly, embodiments of the present disclosure include a method of producing a sensitivity profile for a plurality of cancer cells from a subject, the method comprising: isolating the plurality of cancer cells from a sample, contacting the plurality of cancer cells with a label, treating a first portion of the plurality of cancer cells with a negative control, treating a second portion of the plurality of cancer cells with a positive control, treating a third portion of the plurality of cancer cells with a profiling peptide comprising a cellular uptake moiety and an Mcl-1 binding domain having the sequence of SEQ ID NO:1 with 0-8 modifications, or a composition comprising a profiling peptide comprising a cellular uptake moiety and an Mcl-1 binding domain having the sequence of SEQ ID NO:1 with 0-8 modifications and a carrier, contacting the first portion, the second portion, and the third portion of the plurality of cancer cells with a dye and analyzing the first portion, the second portion, and the third portion of the plurality of cancer cells by flow cytometry.

Some methods described herein further comprise determining an Mcl-1 dependency percentage for the first portion of the plurality of cancer cells based at least on the percent polarization of the first portion and the second portion of the plurality of cancer cells.

In embodiments, polarized and depolarized cells are counted. The percent polarization can then be calculated by dividing the number of polarized cells by the total number of cells and multiplying by 100. In such embodiments, blasts are gated as described above. The blast gate in then plotted on a histogram for detection agent signal. A gate is then created specifically on the polarized cells, and the percent of cells which respond to treatment with a profiling peptide can be found with the following equation:

$$Percent Priming = ( \frac{NC - PP}{NC} ) \times 100 \qquad ( 1 )$$

NC is the average percent polarization of the second portion of the plurality of cancer cells and PP is the percent polarization of the first portion of the plurality of cancer cells.

In embodiments, the Mcl-1 dependency percentage (MDP) is defined by the following equation:

MDP=C×Avg [Percent Priming] (2)
where C is a calibration factor and Avg[Percent Priming] is the average of the percent priming for one or more replicates.

In embodiments, the calibration factor ranges from about 0.1 to about 3.0. In some embodiments, the calibration factor ranges from about 0.5 to about 2.5. In some embodiments, the calibration factor ranges from about 1.0 to about 2.0. In some embodiments, the calibration factor ranges from about 1.4 to about 1.8. In some embodiments, the calibration factor is about 1.5.

Unless otherwise noted, the Mcl-1 dependency percentages calculated herein correspond to a profiling peptide concentration of 1 μM with CCCP as the positive control and water or DMSO as the negative control. In some embodiments, the time occurs over a window from between about 0 to about 300 min to about 0 to about 30 min.

In an illustrative method of the disclosure, a plurality of cancer cells is prepared, and sample quality is confirmed. In the case of a frozen sample, samples are quickly thawed, for example, by placing them in a 37° C. water bath for about 60-70 seconds. After thawing the sample, the cells are transferred to a flask containing warm culture medium. After incubation, the quality (e.g., viability, cell count, etc.) is confirmed. In the case of a fresh sample, mononuclear cells are isolated from bone marrow aspirates following standard laboratory protocol (e.g., Ficoll-Paque separation). The cells are counted and the viability of the isolated cells is determined. The cells are then transferred to a flask containing warm culture medium. The cells are then pelleted and resuspended in a buffer that blocks FC receptors. A mix of monoclonal antibodies is then added and incubated. After incubating with the label, the cells are again pelleted. The cells are again resuspended in a mix of an assay buffer that has a pH ranging from about 7.4 to about 7.6, a diterpenoid (e.g., ryanodine), and an ATP synthase inhibitor (e.g. Oligomycin A). The suspended cells are then aliquoted into three portions. The first portion is mixed with a negative control (e.g., nuclease free water), the second portion is mixed with a positive control (e.g., CCCP), and the third portion is mixed with the profiling peptide (e.g., having the sequence of SEQ ID NO:14). The three portions are then incubated. After the incubation, the cells are pelleted and resuspended in the assay buffer. A dye (e.g., DiOC₆) is then added to each portion of cells.

In the illustrative method, the cells are analyzed via flow cytometry. The control cell lines are analyzed using the live gate. The sample is analyzed by gating single, live cells by plotting forward vs. side scatter. Outliers containing high forward and side scatter values may be assumed to be doublets and dying cells, respectively, and may be excluded from the final analysis. Events that are very low in both forward and side scatter may also be excluded as cellular debris. Generate a dot plot of channel FL5 (PC7 labeled anti-CD45 antibody) against side scatter and identify the CD45 dim population. Using only the events within the “CD45 dim” gate, generate a dot plot of channel FL4 (PC5 labeled anti-CD13, anti-CD33 and anti-CD34 antibodies) against channel FL3 (ECD/PE-Texas Red labeled anti-CD3 and anti-CD20 antibodies). In order to gate exclusively on AML blasts, gate cells that are high in channel FL4 and low in channel FL3 (live cell population). Using only events within the “Blasts” population, generate a histogram of FL1. Determine the cells high in channel FL1. Apply the gates created to all aliquots of all treatments of the same sample.

In other embodiments, the MDP is defined by the following equation:

$$MDP = [ 1 - ( \frac{Pep - PC}{NC - PC} ) ] * 100 \qquad ( 3 )$$

Where PC is the AUC of the positive control, NC is the AUC of the negative control, and Pep is the AUC of the profiling peptide. Unless otherwise noted, the MCL-1 dependency percentages calculated herein correspond to a profiling peptide concentration of 1 μM with CCCP as the positive control and water or DMSO as the negative control. The AUC is either area under the curve or signal intensity. In embodiments, the AUC is the median fluorescent intensity (MFI). In some embodiments, the area under the curve is established by homogenous time-resolved fluorescence (HTRF). In some embodiments, the time occurs over a window from between about 0 to about 300 min to about 0 to about 30 min. In some embodiments, the area under the curve is established by fluorescence activated cell sorting (FACS) or microplate assay as known in the art or described herein. In some embodiments, the signal intensity is a single time point measurement that occurs between about 5 min and about 300 min.

In an illustrative method of the disclosure, a plurality of cancer cells are isolated from a subject sample, and sample quality is confirmed. The cells are then pelleted, blocked in BSA, and labeled. After staining, cells are pelleted and separated into three portions and treated with either water or dimethyl sulfoxide (DMSO) (negative control), CCCP (positive control) or a profiling peptide of the disclosure (subject dependency). DiOC6, a cationic mitochondrial dye is added. Later, the cells are analyzed via flow cytometry.

In some embodiments, a plurality of cancer cells are isolated from primary bone marrow aspirates and sample quality is determined. Cells are then pelleted, blocked in BSA and labeled for markers specific to B and T cells, as well as monocyte differentiation markers and blast-specific markers. After staining, cells are pelleted and separated into three portions and treated with either water (negative control), CCCP (positive control) or SEQ ID NO:14 (subject dependency). DiOC6, a cationic mitochondrial dye is added. The cells are analyzed via flow cytometry. Blast cells are isolated by gating on the CD45 dim, CD13, CD33, and CD34 high population of each sample.

In particular embodiments, a plurality of cancer cells is isolated from primary bone marrow aspirates using density-gradient centrifugation. Sample quality is determined using trypan blue exclusion. Cells are then pelleted, blocked in BSA and labeled for markers specific to B and T cells, as well as monocyte differentiation markers and blast-specific markers. After staining, cells are pelleted and separated into fluorescence-activated cell sorting (FACS) tubes and treated with either water (negative control), CCCP (positive control) or SEQ ID NO:14 (subject dependency). DiOC6, a cationic mitochondrial dye is added. The cells are then analyzed via flow cytometry. Blast cells are isolated by gating on the CD45 dim, CD13, CD33 and CD34 high population of each sample.

In any of the above embodiments, the cancer cell, the plurality of cancer cells, or a portion thereof, is not permeabilized, for example with a cell permeabilization agent such as digitonin.

### II. Kits and Other Dosage Forms

The present disclosure further provides for kits comprising the therapeutic agents (e.g., a CDK inhibitor and an anthracycline) and written instructions for administration of the same. In some embodiments, the kit comprises an effective amount of a CDK inhibitor, an effective amount of an anthracycline, and written instructions for administration of the CDK inhibitor and the anthracycline. In other embodiments, the kit comprises an effective amount of alvocidib, or a prodrug thereof, an effective amount of daunorubicin, and an effective amount of cytarabine, and written instructions for administration of the alvocidib, or a prodrug thereof, the daunorubicin and the cytarabine according to methods of the disclosure for treatment of a hematological cancer, such as AML.

In one embodiment, the kit comprises alvocidib, or a prodrug thereof, or a pharmaceutically acceptable salt of the foregoing; daunorubicin or idarubicin, or a pharmaceutically acceptable salt of the foregoing; cytarabine, or a pharmaceutically acceptable salt thereof; and written instructions for administering the alvocidib, or a prodrug thereof, or a pharmaceutically acceptable salt of the foregoing, daunorubicin or idarubicin, or a pharmaceutically acceptable salt of the foregoing, and cytarabine, or a pharmaceutically acceptable salt thereof, to a subject in need of treatment for a hematologic cancer, such as a hematologic cancer described herein (e.g., AML). In a more specific embodiment, the kit comprises alvocidib, or a pharmaceutically acceptable salt thereof; daunorubicin, or a pharmaceutically acceptable salt thereof; and cytarabine, or a pharmaceutically acceptable salt thereof. In another specific embodiment, the kit comprises alvocidib, or a pharmaceutically acceptable salt thereof; daunorubicin or idarubicin, or a pharmaceutically acceptable salt of the foregoing; and cytarabine, or a pharmaceutically acceptable salt thereof. In yet another specific embodiment, the kit comprises alvocidib, or a pharmaceutically acceptable salt thereof; idarubicin, or a pharmaceutically acceptable salt thereof; and cytarabine, or a pharmaceutically acceptable salt thereof. In yet another specific embodiment, the kit comprises a prodrug of alvocidib (e.g., a compound of Structural Formula I), or a pharmaceutically acceptable salt thereof; daunorubicin or idarubicin, or a pharmaceutically acceptable salt of the foregoing; and cytarabine, or a pharmaceutically acceptable salt thereof. In yet another specific embodiment, the kit comprises a prodrug of alvocidib (e.g., a compound of Structural Formula I), or a pharmaceutically acceptable salt thereof; daunorubicin, or a pharmaceutically acceptable salt thereof; and cytarabine, or a pharmaceutically acceptable salt thereof.

In various embodiments, the written instructions may include instructions regarding dosage, method of administration, order and timing of administration, and the like. The written instructions can be in the form of printed instructions provided within the kit, or the written instructions can be printed on a portion of the container housing the kit. Written instructions may be in the form of a sheet, pamphlet, brochure, CD-ROM, or computer-readable device, or can provide directions to locate instructions at a remote location, such as a website. The written instructions may be in English and/or in a national or regional language.

Kits can further comprise one or more syringes, ampules, vials, tubes, tubing, facemask, a needleless fluid transfer device, an injection cap, sponges, sterile adhesive strips, Chloraprep, gloves, and the like. Variations in contents of any of the kits described herein can be made. In various embodiments, content of the kit is provided in a compact container.

In some embodiments, pharmaceutical compositions of the disclosure are presented in a pack or dispenser device, such as an FDA approved kit, which contain one or more unit dosage forms containing the active ingredient(s). The pack may, for example, comprise metal or plastic foil, such as a blister pack.

In embodiments, the kit (e.g. a pack or dispenser) may be accompanied by a notice associated with the container in a form prescribed by a governmental agency regulating the manufacture, use or sale of pharmaceuticals, which notice is reflective of approval by the agency of the form of the compositions or of human or veterinary administration, in addition to instructions for administration. Such notice, for example, may be of the labeling approved by the U.S. Food and Drug Administration for prescription drugs or of an approved product insert.

Also provided herein are separate dosage forms of the therapeutic agents, wherein the therapeutic agents are associated with one another. The term “associated with one another,” as used herein, means that the separate dosage forms are packaged together or otherwise attached to one another such that it is readily apparent that the separate dosage forms are intended to be sold and administered together (e.g., as part of a treatment regimen, consecutively or simultaneously).

In some embodiments, the dosage form comprises a CDK inhibitor (e.g., alvocidib, or a prodrug thereof, or a pharmaceutically acceptable salt of the foregoing); an anthracycline (e.g., daunorubicin or idarubicin, or a pharmaceutically acceptable salt of the foregoing); and a nucleoside analog (e.g., cytarabine, or a pharmaceutically acceptable salt thereof). In a more specific embodiment, the dosage form comprises alvocidib, or a pharmaceutically acceptable salt thereof; daunorubicin, or a pharmaceutically acceptable salt thereof; and cytarabine, or a pharmaceutically acceptable salt thereof. In another specific embodiment, the dosage form comprises alvocidib, or a pharmaceutically acceptable salt thereof; daunorubicin or idarubicin, or a pharmaceutically acceptable salt of the foregoing; and cytarabine, or a pharmaceutically acceptable salt thereof. In yet another specific embodiment, the dosage form comprises alvocidib, or a pharmaceutically acceptable salt thereof; idarubicin, or a pharmaceutically acceptable salt thereof; and cytarabine, or a pharmaceutically acceptable salt thereof. In yet another specific embodiment, the dosage form comprises a prodrug of alvocidib (e.g., a compound of Structural Formula I), or a pharmaceutically acceptable salt thereof; daunorubicin or idarubicin, or a pharmaceutically acceptable salt of the foregoing; and cytarabine, or a pharmaceutically acceptable salt thereof. In yet another specific embodiment, the dosage form comprises a prodrug of alvocidib (e.g., a compound of Structural Formula I), or a pharmaceutically acceptable salt thereof; daunorubicin, or a pharmaceutically acceptable salt thereof; and cytarabine, or a pharmaceutically acceptable salt thereof.

### EXAMPLE EMBODIMENTS OF THE DISCLOSURE

### Embodiment 1

A method for treating a hematologic cancer in a subject in need thereof, the method comprising administering to the subject a treatment comprising an effective amount of each of:
alvocidib, or a prodrug thereof, or a pharmaceutically acceptable salt of the foregoing;
daunorubicin or idarubicin, or a pharmaceutically acceptable salt of the foregoing; and
cytarabine, or a pharmaceutically acceptable salt thereof.

### Embodiment 2

The method of Embodiment 1, wherein the treatment results in the subject being measurable residual disease (MRD)-negative.

### Embodiment 3

The method of Embodiment 1 or 2, wherein the treatment results in complete remission in the subject.

### Embodiment 4

The method of any one of Embodiments 1-3, wherein the hematologic cancer is multiple myeloma, myelodysplastic syndrome, acute myeloid leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma.

### Embodiment 5

The method of Embodiment 4, wherein the hematologic cancer is AML.

### Embodiment 6

The method of Embodiment 5, wherein the AML is intermediate-risk AML or high-risk AML.

### Embodiment 7

The method of Embodiment 5, wherein the AML is low-risk AML.

### Embodiment 8

The method of any one of Embodiments 5-7, wherein the AML is MCL-1 dependent AML.

### Embodiment 9

The method of any one of Embodiments 1-8, wherein the hematologic cancer is previously untreated.

### Embodiment 10

The method of any one of Embodiments 1-9, wherein the subject does not have core binding factor AML.

### Embodiment 11

The method of any one of Embodiments 1-10, wherein the subject does not have acute promyelocytic leukemia.

### Embodiment 12

The method of any one of Embodiments 1-11, wherein the subject is young.

### Embodiment 13

The method of any one of Embodiments 1-12, wherein the subject is fit.

### Embodiment 14

The method of any one of Embodiments 1-13, comprising administering an effective amount of each of:
alvocidib, or a prodrug thereof, or a pharmaceutically acceptable salt of the foregoing;
daunorubicin, or a pharmaceutically acceptable salt thereof; and
cytarabine, or a pharmaceutically acceptable salt thereof.

### Embodiment 15

The method of any one of Embodiments 1-13, comprising administering an effective amount of each of:
alvocidib, or a pharmaceutically acceptable salt thereof;
daunorubicin or idarubicin, or a pharmaceutically acceptable salt of the foregoing; and
cytarabine, or a pharmaceutically acceptable salt thereof.

### Embodiment 16

The method of any one of Embodiments 1-13, comprising administering an effective amount of each of:
alvocidib, or a pharmaceutically acceptable salt thereof;
daunorubicin, or a pharmaceutically acceptable salt thereof; and
cytarabine, or a pharmaceutically acceptable salt thereof.

### Embodiment 17

The method of any one of Embodiments 1-13, comprising administering an effective amount of each of:
a prodrug of alvocidib, or a pharmaceutically acceptable salt thereof;
daunorubicin or idarubicin, or a pharmaceutically acceptable salt of the foregoing; and
cytarabine, or a pharmaceutically acceptable salt thereof.

### Embodiment 18

The method of any one of Embodiments 1-13, comprising administering an effective amount of each of:
a prodrug of alvocidib, or a pharmaceutically acceptable salt thereof;
daunorubicin, or a pharmaceutically acceptable salt thereof; and
cytarabine, or a pharmaceutically acceptable salt thereof.

### Embodiment 19

The method of any one of Embodiments 1-14, 17 and 18, wherein the prodrug of alvocidib is represented by the following structural formula:

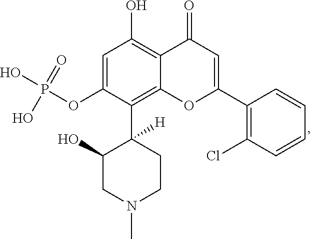 [SMILES: CN1CC[C@H](c2c(OP(=O)(O)O)cc(O)c3c(=O)cc(-c4ccccc4Cl)oc23)[C@H](O)C1]

or a pharmaceutically acceptable salt thereof.

### Embodiment 20

The method of any one of Embodiments 1-19, wherein alvocidib, or a prodrug thereof, or a pharmaceutically acceptable salt of the foregoing, is administered sequentially to daunorubicin or idarubicin, or a pharmaceutically acceptable salt of the foregoing, and cytarabine, or a pharmaceutically acceptable salt thereof.

### Embodiment 21

The method of any one of Embodiments 1-20, wherein alvocidib, or a prodrug thereof, or a pharmaceutically acceptable salt of the foregoing, is administered before each of daunorubicin or idarubicin, or a pharmaceutically acceptable salt of the foregoing, and cytarabine, or a pharmaceutically acceptable salt thereof.

### Embodiment 22

The method of any one of Embodiments 1-21, wherein: alvocidib, or a prodrug thereof, or a pharmaceutically acceptable salt of the foregoing, is

administered to the subject on the first, second and third days of the treatment; daunorubicin or idarubicin, or a pharmaceutically acceptable salt of the foregoing, is

administered to the subject on the fifth, sixth and seventh days of the treatment; and cytarabine, or a pharmaceutically acceptable salt thereof, is administered to the subject on

the fifth, sixth, seventh, eighth, ninth, tenth, and eleventh days of the treatment.

### Embodiment 23

The method of any one of Embodiments 1-22, comprising administering to the subject from about 45 mg/m² to about 110 mg/m² daunorubicin, or a pharmaceutically acceptable salt thereof, per day.

### Embodiment 24

The method of Embodiment 23, comprising administering to the subject about 60 mg/m² daunorubicin, or a pharmaceutically acceptable salt thereof, per day.

### Embodiment 25

The method of any one of Embodiments 1-24, wherein daunorubicin, or a pharmaceutically acceptable salt thereof, is administered to the subject intravenously.

### Embodiment 26

The method of Embodiment 25, wherein daunorubicin, or a pharmaceutically acceptable salt thereof, is administered to the subject via an intravenous bolus.

### Embodiment 27

The method of Embodiment 26, wherein daunorubicin, or a pharmaceutically acceptable salt thereof, is administered to the subject via an intravenous bolus of from about 5 minutes to about 30 minutes in duration.

### Embodiment 28

The method of any one of Embodiments 1-27, comprising administering to the subject from about 90 mg/m² to about 110 mg/m² cytarabine, or a pharmaceutically acceptable salt thereof, per day.

### Embodiment 29

The method of Embodiment 28, comprising administering to the subject about 100 mg/m² cytarabine, or a pharmaceutically acceptable salt thereof, per day.

### Embodiment 30

The method of any one of Embodiments 1-29, wherein cytarabine, or a pharmaceutically acceptable salt thereof, is administered to the subject intravenously.

### Embodiment 31

The method of Embodiment 30, wherein cytarabine, or a pharmaceutically acceptable salt thereof, is administered to the subject via intravenous infusion.

### Embodiment 32

The method of Embodiment 31, wherein cytarabine, or a pharmaceutically acceptable salt thereof, is administered to the subject via intravenous infusion of from about 20 hours to about 28 hours in duration.

### Embodiment 33

The method of any one of Embodiments 1-16 and 20-32, comprising administering to the subject from about 10 mg/m² to about 100 mg/m² alvocidib, or a pharmaceutically acceptable salt thereof, per day.

### Embodiment 34

The method of Embodiment 33, comprising administering to the subject from about 50 mg/m² to about 90 mg/m² alvocidib, or a pharmaceutically acceptable salt thereof, per day.

### Embodiment 35

The method of Embodiment 33, comprising administering to the subject about 50 mg/m² alvocidib, or a pharmaceutically acceptable salt thereof, per day.

### Embodiment 36

The method of Embodiment 33, comprising administering to the subject about 90 mg/m² alvocidib, or a pharmaceutically acceptable salt thereof, per day.

### Embodiment 37

The method of any one of Embodiments 1-16 and 20-36, wherein alvocidib, or a pharmaceutically acceptable salt thereof, is administered to the subject intravenously.

### Embodiment 38

The method of Embodiment 37, wherein alvocidib, or a pharmaceutically acceptable salt thereof, is administered to the subject by intravenous bolus.

### Embodiment 39

The method of Embodiment 38, wherein alvocidib, or a pharmaceutically acceptable salt thereof, is administered to the subject by intravenous bolus of from about 10 minutes to about 60 minutes in duration.

### Embodiment 40

The method of any one of Embodiments 37-39, wherein alvocidib, or a pharmaceutically acceptable salt thereof, is administered to the subject by intravenous infusion.

### Embodiment 41

The method of Embodiment 40, wherein alvocidib, or a pharmaceutically acceptable salt thereof, is administered to the subject by intravenous infusion of from about 3 hours to about 5 hours in duration.

### Embodiment 42

The method of Embodiment 37, wherein: alvocidib, or a pharmaceutically acceptable salt thereof, is administered to the subject via

an intravenous bolus of from about 10 minutes to about 60 minutes in duration; and alvocidib, or a pharmaceutically acceptable salt thereof, is administered to the subject via

intravenous infusion of from about 3 hours to about 5 hours in duration.

### Embodiment 43

The method of Embodiment 42, wherein the intravenous infusion of alvocidib, or a pharmaceutically acceptable salt thereof, is initiated within about one hour of completion of the intravenous bolus of alvocidib, or a pharmaceutically acceptable salt thereof.

### Embodiment 44

The method of Embodiment 43, wherein the intravenous infusion of alvocidib, or a pharmaceutically acceptable salt thereof, is initiated about 30 minutes after completion of the intravenous bolus of alvocidib, or a pharmaceutically acceptable salt thereof.

### Embodiment 45

The method of any one of Embodiments 37-44, wherein from about 25 mg/m² to about 60 mg/m² alvocidib, or a pharmaceutically acceptable salt thereof, is administered to the subject by an intravenous bolus of from about 10 minutes to about 60 minutes in duration.

### Embodiment 46

The method of Embodiment 45, wherein about 50 mg/m² alvocidib, or a pharmaceutically acceptable salt thereof, is administered to the subject by an intravenous bolus of from about 15 minutes to about 45 minutes in duration.

### Embodiment 47

The method of any one of Embodiments 37-44, wherein from about 5 mg/m² to about 50 mg/m² alvocidib, or a pharmaceutically acceptable salt thereof, is administered to the subject by an intravenous bolus of from about 10 minutes to about 60 minutes in duration.

### Embodiment 48

The method of Embodiment 47, wherein from about 25 mg/m² to about 35 mg/m² alvocidib, or a pharmaceutically acceptable salt thereof, is administered to the subject by an intravenous bolus of from about 15 minutes to about 45 minutes in duration.

### Embodiment 49

The method of any one of Embodiments 37-48, wherein from about 5 mg/m² to about 75 mg/m² alvocidib, or a pharmaceutically acceptable salt thereof, is administered to the subject by intravenous infusion of from about 3 hours to about 5 hours in duration.

### Embodiment 50

The method of Embodiment 49, wherein from about 10 mg/m² to about 65 mg/m² alvocidib, or a pharmaceutically acceptable salt thereof, is administered to the subject by intravenous infusion of about 4 hours in duration.

### Embodiment 51

The method of Embodiment 37, wherein:
from about 25 mg/m² to about 35 mg/m² alvocidib, or a pharmaceutically acceptable salt thereof, is administered to the subject by an intravenous bolus of from about 15 minutes to about 45 minutes in duration; and
from about 10 mg/m² to about 65 mg/m² alvocidib, or a pharmaceutically acceptable salt thereof, is administered to the subject by intravenous infusion of about 4 hours in duration.

### Embodiment 52

The method of Embodiment 51, wherein the intravenous infusion of alvocidib, or a pharmaceutically acceptable salt thereof, is initiated within about one hour of completion of the intravenous bolus of alvocidib, or a pharmaceutically acceptable salt thereof.

### Embodiment 53

The method of Embodiment 52, wherein the intravenous infusion of alvocidib, or a pharmaceutically acceptable salt thereof, is initiated about 30 minutes after completion of the intravenous bolus of alvocidib, or a pharmaceutically acceptable salt thereof.

### Embodiment 54

The method of any one of Embodiments 1-14 and 17-45, wherein the prodrug of alvocidib, or a pharmaceutically acceptable salt thereof, is administered to the subject orally.

### Embodiment 55

The method of any one of Embodiments 1-21, comprising a first treatment, wherein:
alvocidib, or a prodrug thereof, or a pharmaceutically acceptable salt of the foregoing, is administered to the subject on the first, second and third days of the first treatment;
daunorubicin or idarubicin, or a pharmaceutically acceptable salt of the foregoing, is administered to the subject on the fifth, sixth and seventh days of the first treatment; and
cytarabine, or a pharmaceutically acceptable salt thereof, is administered to the subject on the fifth, sixth, seventh, eighth, ninth, tenth, and eleventh days of the first treatment; and
a second treatment, wherein:
alvocidib, or a prodrug thereof, or a pharmaceutically acceptable salt of the foregoing, is administered to the subject on the first, second and third days of the second treatment;
daunorubicin or idarubicin, or a pharmaceutically acceptable salt of the foregoing, is administered to the subject on the fifth and sixth days of the second treatment; and
cytarabine, or a pharmaceutically acceptable salt thereof, is administered to the subject on the fifth, sixth, seventh, eighth and ninth days of the second treatment.

### Embodiment 56

The method of Embodiment 55, comprising administering to the subject from about 45 mg/m² to about 110 mg/m² daunorubicin, or a pharmaceutically acceptable salt thereof, per day during the first treatment.

### Embodiment 57

The method of Embodiment 56, comprising administering to the subject about 60 mg/m² daunorubicin, or a pharmaceutically acceptable salt thereof, per day during the first treatment.

### Embodiment 58

The method of any one of Embodiments 55-57, comprising administering to the subject from about 30 mg/m² to about 60 mg/m² daunorubicin, or a pharmaceutically acceptable salt thereof, per day during the second treatment.

### Embodiment 59

The method of Embodiment 58, comprising administering to the subject about 45 mg/m² daunorubicin, or a pharmaceutically acceptable salt thereof, per day during the second treatment.

### Embodiment 60

The method of any one of Embodiments 55-59, wherein daunorubicin, or a pharmaceutically acceptable salt thereof, is administered to the subject intravenously.

### Embodiment 61

The method of Embodiment 60, wherein daunorubicin, or a pharmaceutically acceptable salt thereof, is administered to the subject by intravenous bolus.

### Embodiment 62

The method of Embodiment 61, wherein daunorubicin, or a pharmaceutically acceptable salt thereof, is administered to the subject by intravenous bolus of from about 5 minutes to about 30 minutes in duration during the first treatment.

### Embodiment 63

The method of any one of Embodiments 60-62, wherein daunorubicin, or a pharmaceutically acceptable salt thereof, is administered to the subject by intravenous bolus of from about 5 minutes to about 30 minutes in duration during the second treatment.

### Embodiment 64

The method of any one of Embodiments 55-63, comprising administering to the subject from about 90 mg/m² to about 110 mg/m² cytarabine, or a pharmaceutically acceptable salt thereof, per day during the first treatment.

### Embodiment 65

The method of Embodiment 64, comprising administering to the subject about 100 mg/m² cytarabine, or a pharmaceutically acceptable salt thereof, per day during the first treatment.

### Embodiment 66

The method of any one of Embodiments 55-65, comprising administering to the subject from about 90 mg/m² to about 110 mg/m² cytarabine, or a pharmaceutically acceptable salt thereof, per day during the second treatment.

### Embodiment 67

The method of Embodiment 66, comprising administering to the subject about 100 mg/m² cytarabine, or a pharmaceutically acceptable salt thereof, per day during the second treatment.

### Embodiment 68

The method of any one of Embodiments 55-67, wherein cytarabine, or a pharmaceutically acceptable salt thereof, is administered to the subject intravenously.

### Embodiment 69

The method of Embodiment 68, wherein cytarabine, or a pharmaceutically acceptable salt thereof, is administered by intravenous infusion.

### Embodiment 70

The method of Embodiment 69, wherein cytarabine, or a pharmaceutically acceptable salt thereof, is administered to the subject by intravenous infusion of from about 20 hours to about 28 hours in duration during the first treatment.

### Embodiment 71

The method of any one of Embodiments 68-70, wherein cytarabine, or a pharmaceutically acceptable salt thereof, is administered to the subject by intravenous infusion of from about 20 hours to about 28 hours in duration during the second treatment.

### Embodiment 72

The method of any one of Embodiments 55-71, comprising administering to the subject from about 10 mg/m² to about 100 mg/m² alvocidib, or a pharmaceutically acceptable salt thereof, per day.

### Embodiment 73

The method of Embodiment 72, comprising administering to the subject from about 50 mg/m² to about 90 mg/m² alvocidib, or a pharmaceutically acceptable salt thereof, per day.

### Embodiment 74

The method of Embodiment 72, comprising administering to the subject about 50 mg/m² alvocidib, or a pharmaceutically acceptable salt thereof, per day.

### Embodiment 75

The method of Embodiment 72, comprising administering to the subject about 90 mg/m² alvocidib, or a pharmaceutically acceptable salt thereof, per day.

### Embodiment 76

The method of any one of Embodiments 55-75, wherein alvocidib, or a pharmaceutically acceptable salt thereof, is administered to the subject intravenously.

### Embodiment 77

The method of Embodiment 76, wherein alvocidib, or a pharmaceutically acceptable salt thereof, is administered to the subject by intravenous bolus.

### Embodiment 78

The method of Embodiment 77, wherein alvocidib, or a pharmaceutically acceptable salt thereof, is administered to the subject by intravenous bolus of from about 10 minutes to about 60 minutes in duration.

### Embodiment 79

The method of any one of Embodiments 76-78, wherein alvocidib, or a pharmaceutically acceptable salt thereof, is administered to the subject by intravenous infusion.

### Embodiment 80

The method of Embodiment 79, wherein alvocidib, or a pharmaceutically acceptable salt thereof, is administered to the subject by intravenous infusion of from about 3 hours to about 5 hours in duration.

### Embodiment 81

The method of Embodiment 76, wherein: alvocidib, or a pharmaceutically acceptable salt thereof, is administered to the subject via

an intravenous bolus of from about 10 minutes to about 60 minutes in duration; and alvocidib, or a pharmaceutically acceptable salt thereof, is administered to the subject via

intravenous infusion of from about 3 hours to about 5 hours in duration.

### Embodiment 82

The method of Embodiment 81, wherein the intravenous infusion of alvocidib, or a pharmaceutically acceptable salt thereof, is initiated within about one hour of completion of the intravenous bolus of alvocidib, or a pharmaceutically acceptable salt thereof.

### Embodiment 83

The method of Embodiment 82, wherein the intravenous infusion of alvocidib, or a pharmaceutically acceptable salt thereof, is initiated about 30 minutes after completion of the intravenous bolus of alvocidib, or a pharmaceutically acceptable salt thereof.

### Embodiment 84

The method of any one of Embodiments 76-83, wherein from about 25 mg/m² to about 60 mg/m² alvocidib, or a pharmaceutically acceptable salt thereof, is administered to the subject by an intravenous bolus of from about 10 minutes to about 60 minutes in duration.

### Embodiment 85

The method of Embodiment 84, wherein about 50 mg/m² alvocidib, or a pharmaceutically acceptable salt thereof, is administered to the subject by an intravenous bolus of from about 15 minutes to about 45 minutes in duration.

### Embodiment 86

The method of any one of Embodiments 76-83, wherein from about 5 mg/m² to about 50 mg/m² alvocidib, or a pharmaceutically acceptable salt thereof, is administered to the subject by an intravenous bolus of from about 10 minutes to about 60 minutes in duration.

### Embodiment 87

The method of Embodiment 86, wherein from about 25 mg/m² to about 35 mg/m² alvocidib, or a pharmaceutically acceptable salt thereof, is administered to the subject by an intravenous bolus of from about 15 minutes to about 45 minutes in duration.

### Embodiment 88

The method of any one of Embodiments 76-87, wherein from about 5 mg/m² to about 75 mg/m² alvocidib, or a pharmaceutically acceptable salt thereof, is administered to the subject by an intravenous infusion of from about 3 hours to about 5 hours in duration.

### Embodiment 89

The method of Embodiment 88, wherein from about 10 mg/m² to about 65 mg/m² alvocidib, or a pharmaceutically acceptable salt thereof, is administered to the subject by an intravenous infusion of about 4 hours in duration.

### Embodiment 90

The method of Embodiment 76, wherein:
from about 25 mg/m² to about 35 mg/m² alvocidib, or a pharmaceutically acceptable salt thereof, is administered to the subject by an intravenous bolus of from about 15 minutes to about 45 minutes in duration; and
from about 10 mg/m² to about 65 mg/m² alvocidib, or a pharmaceutically acceptable salt thereof, is administered to the subject by intravenous infusion of about 4 hours in duration.

### Embodiment 91

The method of Embodiment 90, wherein the intravenous infusion of alvocidib, or a pharmaceutically acceptable salt thereof, is initiated within about one hour of completion of the intravenous bolus of alvocidib, or a pharmaceutically acceptable salt thereof.

### Embodiment 92

The method of Embodiment 91, wherein the intravenous infusion of alvocidib, or a pharmaceutically acceptable salt thereof, is initiated about 30 minutes after completion of the intravenous bolus of alvocidib, or a pharmaceutically acceptable salt thereof.

### Embodiment 93

The method of any one of Embodiments 1-92, further comprising detecting the MRD status of the subject.

### Embodiment 94

The method of Embodiment 93, wherein the MRD status of the subject is detected prior to administering the treatment to the subject.

### Embodiment 95

The method of Embodiment 93, wherein the MRD status of the subject is detected after administering the treatment to the subject.

### Embodiment 96

The method of Embodiment 93, wherein the MRD status of the subject is detected prior to and after administering the treatment to the subject.

### Embodiment 97

The method of any one of Embodiments 1-96, further comprising terminating the treatment when detection of the subject's MRD status reveals that the subject is MRD-negative.

### Embodiment 98

The method of any one of Embodiments 1-97, wherein the treatment results in the subject being measurable residual disease (MRD)-negative and results in complete remission in the subject.

### Embodiment 99

A method for treating previously untreated acute myeloid leukemia (AML) in a subject in need thereof, the method comprising administering to the subject a treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment,
wherein the treatment results in the subject being measurable residual disease (MRD)-negative.

### Embodiment 100

The method of Embodiment 99, comprising administering to the subject a treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
about 60 mg/m² daunorubicin, or a pharmaceutically acceptable salt thereof, per day, administered by intravenous bolus of from about 5 minutes to about 30 minutes in duration on the fifth, sixth and seventh days of the treatment; and
about 100 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment.

### Embodiment 101

The method of Embodiment 99 or 100, wherein the treatment results in complete remission in the subject.

### Embodiment 102

The method of any one of Embodiments 99-101, comprising administering to the subject one cycle of the treatment.

### Embodiment 103

The method of any one of Embodiments 99-101, comprising administering to the subject a first treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the first treatment, and the intravenous infusion is initiated about 30 minutes after completion of the intravenous bolus;
from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the first treatment; and from about 90 mg/m² to about 110 mg/m² cytarabine, or a pharmaceutically acceptable salt
thereof, per day, administered by intravenous infusion of from about 20 hours to about 28 hours in duration on the fifth, sixth, seventh, eighth, ninth, tenth, and eleventh days of the first treatment; and
   a second treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the second treatment, and the intravenous infusion is initiated about 30 minutes after completion of the intravenous bolus;
from about 30 mg/m² to about 60 mg/m² daunorubicin, or a pharmaceutically acceptable salt thereof, per day, administered by intravenous bolus of from about 5 minutes to about 30 minutes in duration on the fifth and sixth days of the second treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth and ninth days of the second treatment,
wherein the first day of the second treatment corresponds to the fifteenth day of the first treatment.

### Embodiment 104

A method for treating intermediate-risk acute myeloid leukemia (AML) or high-risk AML in a subject in need thereof, the method comprising administering to the subject a treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment.

### Embodiment 105

The method of Embodiment 104, wherein the treatment results in the subject being measurable residual disease (MRD)-negative.

### Embodiment 106

The method of Embodiment 104 or 105, comprising administering to the subject a treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
about 60 mg/m² daunorubicin, or a pharmaceutically acceptable salt thereof, per day, administered by intravenous bolus of from about 5 minutes to about 30 minutes in duration on the fifth, sixth and seventh days of the treatment; and
about 100 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment.

### Embodiment 107

The method of any one of Embodiments 104-106, wherein the treatment results in complete remission in the subject.

### Embodiment 108

The method of any one of Embodiments 104-107, comprising administering to the subject one cycle of the treatment.

### Embodiment 109

The method of any one of Embodiments 104-107, comprising administering to the subject a first treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the first treatment, and the intravenous infusion is initiated about 30 minutes after completion of the intravenous bolus;
from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the first treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the first treatment; and
   a second treatment comprising:
from about 5 mg/m2 to about 50 mg/m2 alvocidib, or a pharmaceutically acceptable salt thereof, per day, administered by an intravenous bolus of from about 10 minutes to about 60 minutes in duration, and from about 10 mg/m2 to about 65 mg/m2 alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the second treatment, and the intravenous infusion is initiated about 30 minutes after completion of the intravenous bolus;
from about 30 mg/m² to about 60 mg/m² daunorubicin, or a pharmaceutically acceptable salt thereof, per day, administered by intravenous bolus of from about 5 minutes to about 30 minutes in duration on the fifth and sixth days of the second treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth and ninth days of the second treatment,
wherein the first day of the second treatment corresponds to the fifteenth day of the first treatment.

### Embodiment 110

The method of Embodiment 109, comprising administering to the subject a first treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the first treatment, and the intravenous infusion is initiated about 30 minutes after completion of the intravenous bolus;
   about 60 mg/m2 daunorubicin, or a pharmaceutically acceptable salt thereof, per day, administered by intravenous bolus of from about 5 minutes to about 30 minutes in duration on the fifth, sixth and seventh days of the first treatment; and about 100 mg/m2 cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the first treatment; and
      a second treatment comprising:
from about 5 mg/m2 to about 50 mg/m2 alvocidib, or a pharmaceutically acceptable salt thereof, per day, administered by an intravenous bolus of from about 10 minutes to about 60 minutes in duration, and from about 10 mg/m2 to about 65 mg/m2 alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the second treatment, and the intravenous infusion is initiated about 30 minutes after completion of the intravenous bolus;
   about 45 mg/m2 daunorubicin, or a pharmaceutically acceptable salt thereof, per day, administered by intravenous bolus of from about 5 minutes to about 30 minutes in duration on the fifth and sixth days of the second treatment; and about 100 mg/m2 cytarabine, or a pharmaceutically acceptable salt thereof, per day, administered by intravenous infusion of from about 20 hours to about 28 hours in duration on the fifth, sixth, seventh, eighth and ninth days of the second treatment,
wherein the first day of the second treatment corresponds to the fifteenth day of the first treatment.

### Embodiment 111

A kit, comprising:
alvocidib, or a prodrug thereof, or a pharmaceutically acceptable salt of the foregoing;
daunorubicin or idarubicin, or a pharmaceutically acceptable salt of the foregoing;
cytarabine, or a pharmaceutically acceptable salt thereof; and
written instructions for administering the alvocidib, or a prodrug thereof, or a pharmaceutically acceptable salt of the foregoing, daunorubicin or idarubicin, or a pharmaceutically acceptable salt of the foregoing, and cytarabine, or a pharmaceutically acceptable salt thereof, to a subject in need of treatment for a hematologic cancer.

### Embodiment 112

The kit of Embodiment 111, wherein the hematologic cancer is acute myeloid leukemia (AML).

### Embodiment 113

The kit of Embodiment 111 or 112, comprising alvocidib, or a pharmaceutically acceptable salt thereof, daunorubicin, or a pharmaceutically acceptable salt thereof, and cytarabine, or a pharmaceutically acceptable salt thereof.

### Embodiment 114

The kit of Embodiment 111 or 112, comprising alvocidib, or a pharmaceutically acceptable salt thereof, daunorubicin or idarubicin, or a pharmaceutically acceptable salt of the foregoing, and cytarabine, or a pharmaceutically acceptable salt thereof.

### Embodiment 115

The kit of Embodiment 111 or 112, comprising alvocidib, or a pharmaceutically acceptable salt thereof, idarubicin, or a pharmaceutically acceptable salt thereof, and cytarabine, or a pharmaceutically acceptable salt thereof.

### Embodiment 116

A dosage form, comprising: alvocidib, or a prodrug thereof, or a pharmaceutically acceptable salt of the foregoing; daunorubicin or idarubicin, or a pharmaceutically acceptable salt of the foregoing; and cytarabine, or a pharmaceutically acceptable salt thereof.

### Embodiment 117

The dosage form of Embodiment 116, comprising alvocidib, or a pharmaceutically acceptable salt thereof, daunorubicin, or a pharmaceutically acceptable salt thereof, and cytarabine, or a pharmaceutically acceptable salt thereof.

### Embodiment 118

The dosage form of Embodiment 116, comprising: a prodrug of alvocidib, or a pharmaceutically acceptable salt thereof; daunorubicin or idarubicin, or a pharmaceutically acceptable salt of the foregoing; and cytarabine, or a pharmaceutically acceptable salt thereof.

### Embodiment 119

The dosage form of Embodiment 118, wherein the prodrug of alvocidib is represented by the following structural formula:

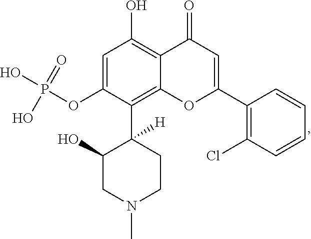 [SMILES: CN1CC[C@H](c2c(OP(=O)(O)O)cc(O)c3c(=O)cc(-c4ccccc4Cl)oc23)[C@H](O)C1]

or a pharmaceutically acceptable salt thereof.

Embodiments of this disclosure are further illustrated by the following non-limiting examples.

### EXAMPLES

### Example 1

### Single Agent Treatment Effect on Activities in AML Cells

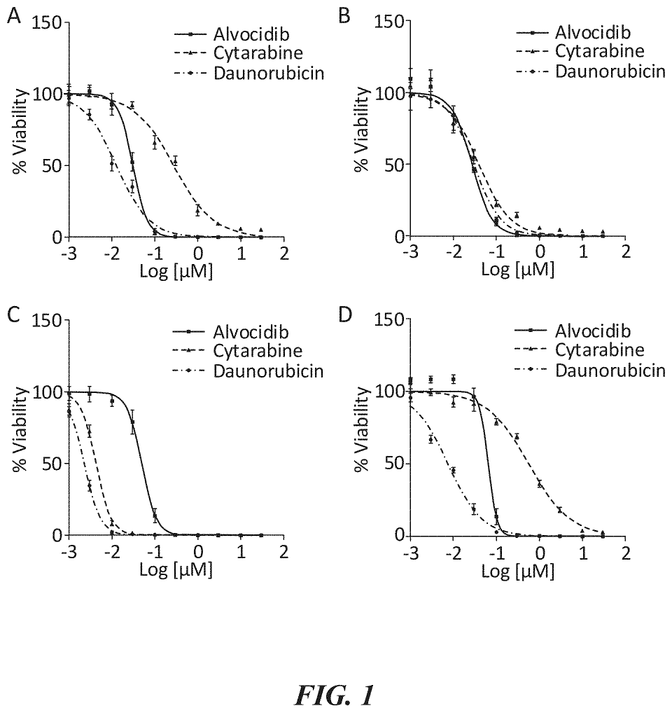

FIG. 1 shows differing activities of alvocidib, cytarabine, and daunorubicin in various AML cell lines. (A) OCI-AML3, (B) U937, (C) Molm-13, and (D) MV4-11 cells were treated with alvocidib, cytarabine, or daunorubicin, as single agents, in viability assays using CellTiter-Glo according to manufacturer protocol. It was observed that the 72-hour viability assays yielded single agent IC₅₀ values of alvocidib, cytarabine, or daunorubicin in cultured AML cells ranging from 2.2 nM to 567 nM. While alvocidib showed similar IC_{50S} across cell lines, cytarabine and daunorubicin showed differing activities in Molm-13, in comparison to OCI-AML3.

### Example 2

### Alvocidib Suppresses MCL-1 Expression Throughout the Treatment Regimen of Cytarabine and Daunorubicin

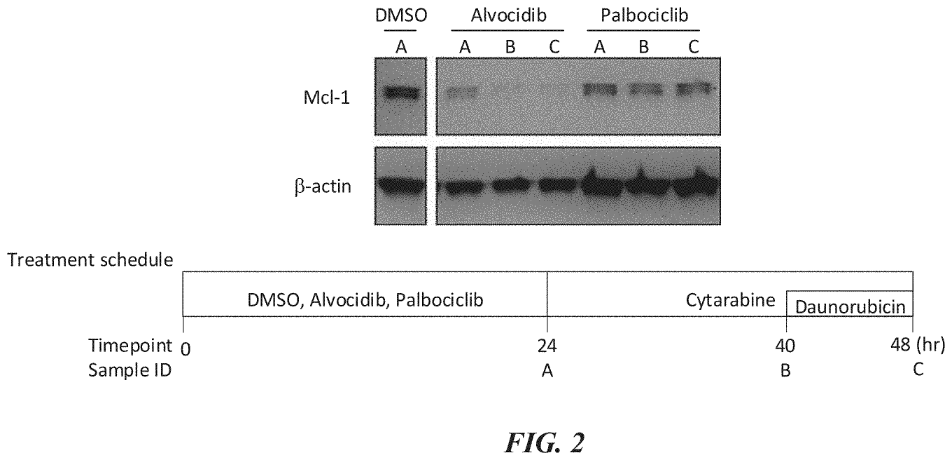

As shown in FIG. 2, alvocidib suppresses MCL-1 expression throughout the treatment regimen of cytarabine and daunorubicin. MCL-1 expression during the alvocidib+cytarabine+daunorubicin regimen was modeled in vitro. MV4-11 cells were treated with alvocidib (80 nM), cytarabine (100 nM), or daunorubicin (3 nM), in the sequence shown. Alvocidib (or palbociclib) was washed out prior to cytarabine addition. Only alvocidib-containing regimens showed sustained MCL-1 knockdown throughout the treatment schedule. Conversely, high concentrations of CDK4/6 (palbociclib, 1 μM) inhibitor did not achieve this same MCL-1 knockdown.

### Example 3

### Synergistic Results of Combination Therapy in Cell Lines

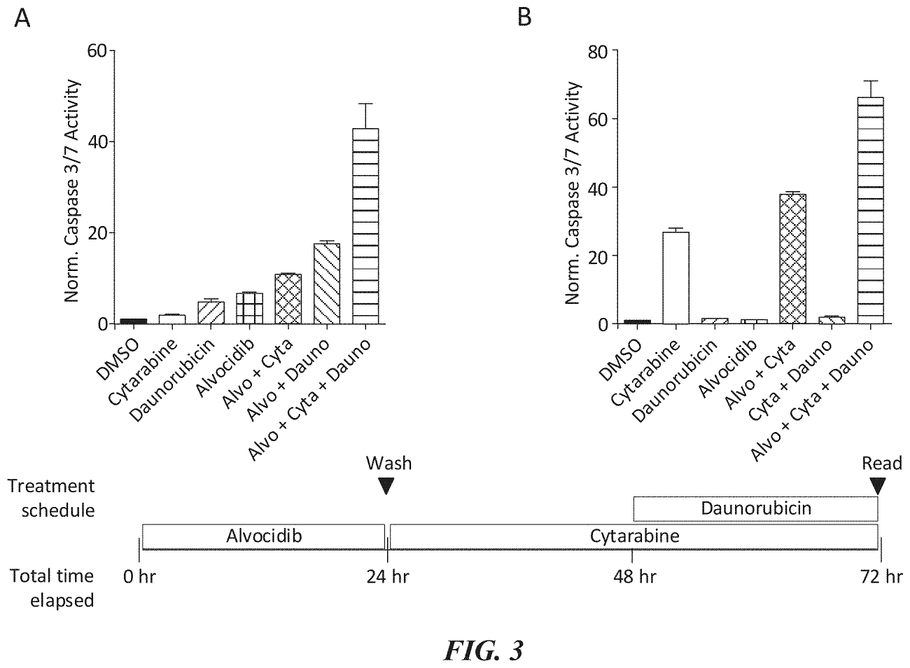

Apoptosis/caspase activity in cells treated with various combinations of drugs, including cytarabine and daunorubicin were assessed using the Caspase-Glo assay (according to manufacturer protocol) in both MV4-11 (A) and U937 (B) cell lines. Concentrations used were: alvocidib at 100 nM, cytarabine at 500 or 50 nM, respectively, and daunorubicin at 10 nM. Drugs tested as single agents, with the exception of cytarabine in U937 cells, failed to induce significant levels of apoptosis at the concentrations tested. However, the alvocidib+cytarabine+daunorubicin combination shows significant synergy in inducing apoptosis in both of these cell lines. As shown in FIG. 3, alvocidib enhances the induction of apoptosis by cytarabine and daunorubicin.

### Example 4

### Synergistic Results of Combination Therapy in AML Xenograft

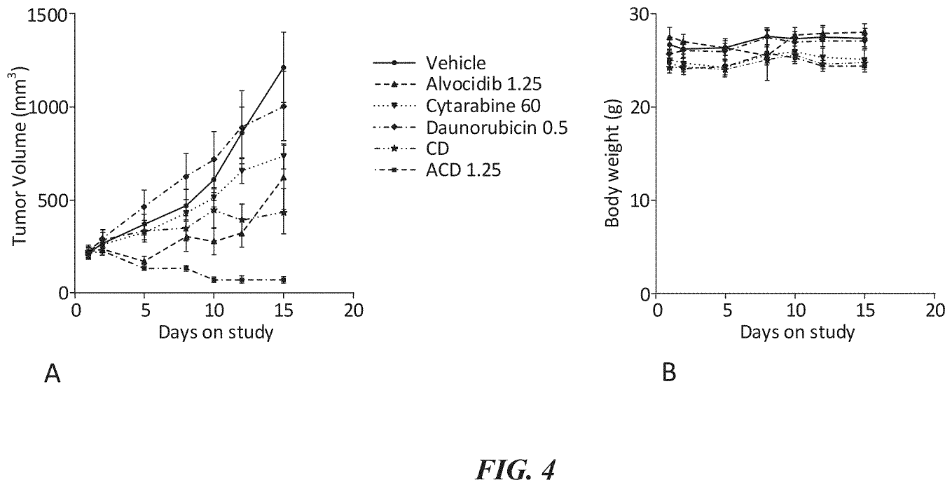

Alvocidib with a cytarabine and daunorubicin regimen were tested in vivo in the MV4-11 AML xenograft model using female athymic nude mice. FIG. 4 shows that alvocidib potentiates the activity of cytarabine and daunorubicin in an AML xenograft.

ACD (alvocidib+cytarabine+daunorubicin) is an active regimen in MV4-11 AML xenografts. (A) Tumor volume measurements show decreases following the sequential dosing of alvocidib (qdx2), cytarabine (qdx2), and daunorubicin (qdx1). This five day cycle was repeated twice. (B) Bodyweight was not affected significantly. Doses indicated in mg/kg.

In the xenograft study, 21.1 and 48.5% tumor growth inhibition (TGI) was observed following either single agent treatment of daunorubicin (0.5 mg/kg) or cytarabine (60 mg/kg), respectively. Treatment of alvocidib (1.25 mg/kg) yielded 60.0% TGI. Combination treatment, however, resulted in tumor shrinkage, yielding 116.2% TGI. The data here demonstrates that alvocidib potentiates the activity of cytarabine and daunorubicin.

### Example 5

### Clinical Trial Design

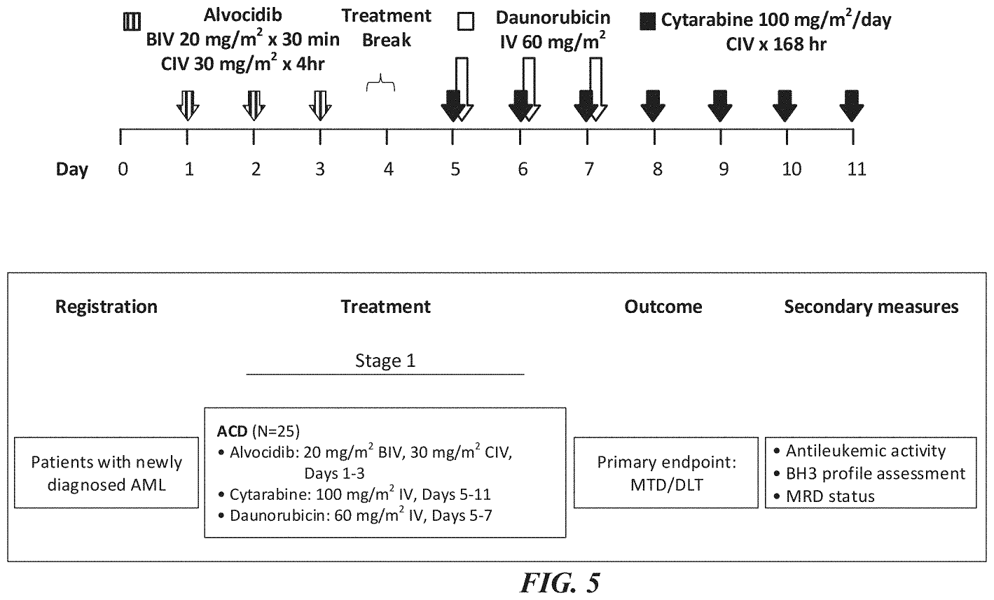

FIG. 5 shows a Phase Ib clinical trial design in patients with newly diagnosed AML. The starting dose of alvocidib is 20 mg/m² as a 30-minute intravenous (IV) bolus followed by 30 mg/m² over 4 hours as an IV infusion administered daily on Days 1-3 of induction. The dose of alvocidib administered in the IV infusion is escalated in 10 mg/m² increments to 60 mg/m² to determine the maximum tolerated dose and to identify any dose limiting toxicity.

Patients have a one day treatment break (Day 4) before initiation of the 7+3 regimen. Beginning on Day 5, cytarabine is administered as a 100 mg/m²/day continuous IV infusion for seven consecutive days (Days 5-11) plus daunorubicin administered at a bolus dosage of 60 mg/m² IV on Days 5-7.

The primary outcome measures of this Phase Ib trial are the maximum tolerated dose and identification of any dose-limiting toxicities (i.e., the dose at which <1 of 6 patients experience a DLT during Cycle 1 with the next higher dose having at least 2 of 3 to 6 patients experiencing a DLT during Cycle 1). Secondary outcome measures include the antileukemic activity of alvocidib plus 7+3, correlation between activity of alvocidib plus 7+3 and mitochondrial BH3 profiling, and assessment of minimal residual disease (MRD).

In some aspects, patients are treated according to the procedures described in this example, and elsewhere within this disclosure, only if their cancer cells have an MCL-1 priming percentage of at least 15% as described herein.

### Example 6

### Illustrative Assay Procedure

### Controls

A single set of controls (one positive and one negative) is included in every run. The controls are treated in the same manner as frozen samples.

### Samples

The assay may be performed on fresh or frozen BMMCs.

Frozen Sample
1. Prepare a T25 flask for each sample with 10 mL of RPMI 1640 culture medium warmed for 15 to 30 minutes in a 37° C. (5% CO₂) incubator. Add 150U of DNase I to each flask.
2. Quickly thaw the samples (frozen BMMCs) by placing them in a 37° C. water bath for ˜60-70 seconds.
3. Immediately after thawing each sample, add the entire contents of the tube (should be at least 2 million cells with at least 40% viability) to one T25 flask containing warmed culture medium, dropwise and with a constant swirling motion of the medium.
4. Incubate in a 37° C. (5% CO₂) incubator for one hour.
5. After one hour of incubation, count the cells and ensure that the sample requirements (at least 2 million cells with at least 40% viability) are still met.

Fresh Sample
1. Isolate mononuclear cells from bone marrow aspirates following standard laboratory protocol. Count the cells and determine the viability of the isolated cells. If viability is >40%, seed 2 million BMMCs in cell culture medium using the procedure below.
2. Prepare a T25 flask with 10 mL of RPMI 1640 culture medium warmed for 15 to 30 minutes in a 37° C. (5% CO2) incubator.
3. Add 2 million of the freshly prepared cells to the T25 flask containing warmed culture medium, dropwise with a constant swirling motion of the medium.
4. Incubate in a 37° C. (5% CO₂) incubator for one hour.

### Cell Blocking and Staining

1. For each sample or control being run, transfer the cells from the T25 flask with RPMI medium into a 15 mL conical tube and spin the cells down at 350×g for 5 minutes at room temperature.
2. Decant the RPMI medium and resuspend the cells in 1 mL of room temperature Blocking Buffer. Pipette gently to mix.
3. Incubate the cells at room temperature for 15 minutes to allow for blocking of the FC receptors.
4. Add 15 μL of the Monoclonal Antibody Mix to the cell suspension and incubate for 30 minutes at room temperature in the dark.

### Signal Stabilizer Treatment

Assay Buffer Master Mix Calculation

Volume of Assay Buffer (mL)=number of (samples+controls)×2 mL
Equation 1: Calculation of the Assay Buffer Volume (mL)

Volume of Signal Stabilizer (in μL)=Volume (in mL) of Assay Buffer needed×1.5
Equation 2: Calculation of Stabilizer Volume (μL)

TABLE 2
Volume of Assay Buffer and Stabilizer

| Number of | Volume of Assay | Volume of |
|---|---|---|
| (Sample + Control) | Buffer (mL) | Stabilizer (μL) |
| 3 | 6 | 9 |
| 4 | 8 | 12 |

### Treatment Procedure

1. Upon completion of the Cell Blocking and Staining incubation, spin down the 15 mL conical tubes at 350×g for 5 minutes at room temperature.
2. For each sample and control, eight 12×75 mm flow tubes are used. Of those eight, 3 will be used for Nuclease-Free Water, 1 will be used for Depolarizing Solution, and 4 will be used for the profiling peptide.

TABLE 3
Number of Replicates.

| | Nuclease-Free | Depolarizing | SEQ ID NO: 14 |
|---|---|---|---|
| | Water (“W”) | Solution (“D”) | (“T”) |
| Number of | 3 | 1 | 4 |
| Replicates | | | |

3. Thaw one tube of Signal Stabilizer (containing Ryanodine and Oligomycin). Make sure it is completely thawed and well mixed before use.
4. Calculate the appropriate volume of Assay Buffer and Signal Stabilizer per the total number of samples and controls. Use Table 2 as a reference.
5. In a separate tube, prepare the Assay Buffer and Signal Stabilizer mix based on the calculations in step 4. Vortex to mix.
6. Decant the Blocking Buffer/Antibody Mix from the pelleted cells. Resuspend each sample and control cell pellet in 2 mL of the master mix (prepared in step 5).
7. Lightly vortex each of the cell suspensions for a few seconds.
8. For each sample and control, aliquot 250 μL of the resultant cell suspension into each of the eight tubes from step 2.
9. Add 2.5 μL Nuclease-Free Water, or 2.5 μL Depolarizing Solution, or 2.5 μL SEQ ID NO:14 Peptide each to their respective tubes.
10. Lightly vortex the tubes for a few seconds.
11. Incubate all tubes at 37° C. (5% CO₂) for 30 minutes.

### Cell Washing and Mitochondrial Staining

1. Once the incubation is complete, spin the tubes of treated cells at 350×g for 5 minutes at room temperature. Decant the supernatant.
2. Resuspend each cell pellet in 500 μL of room temperature Assay Buffer. Spin down the cells at 350×g for 5 minutes at room temperature.
3. Thaw one tube of Mitochondrial Staining Solution. Make sure it is completely thawed and well mixed before use.
4. During centrifugation, prepare the working dye solution in Assay Buffer by mixing 1 of Mitochondrial Staining Solution stock for every 1 mL of Assay Buffer needed. Vortex to mix.

TABLE 4
Volume for Assay Buffer and Mitochondrial Staining Solution,

| Number of | Volume of | Volume of | Volume of |
|---|---|---|---|
| (Samples + | Assay Buffer | Working Dye | Mitochondrial |
| Controls) | (mL) | Solution (mL) | Staining Solution (μL) |
| 3 | 12 | 12 | 12 |
| 4 | 16 | 16 | 16 |

5. After the spin is complete, decant the Assay Buffer. Resuspend each cell pellet in 500 μL of the working dye solution.
6. Lightly vortex the tubes for a few seconds.
7. Incubate all tubes at 37° C. (5% CO₂) for 90 minutes.
8. Proceed promptly to Data Acquisition, when the incubation is complete.

### Data Acquisition

Data can be acquired on a Beckman Coulter FC500 Flow Cytometer using a 488 nm laser. The wavelengths detected are listed in Table 5.

TABLE 5
List of detection wavelengths and their common fluorophores.

| Channel | Wavelength Detected | Common Fluorophores/Channel Names |
|---|---|---|
| FL1 | 525 nm/40 | FITC |
| FL2 | 575 nm/40 | PE |
| FL3 | 620 nm/20 | ECD/PE-Texas Red |
| FL4 | 675 nm/40 | PC5 |
| FL5 | 755 nm/40 | PC7 |

### Compensation Acquisition

Perform the compensation according to the manufacturer's protocol.

### Sample Acquisition

Acquire data on the controls before the samples.
1. For each control and sample set, load the tubes onto the carousel in the following order:
   a. Water
   b. Depolarizing Solution
   c. SEQ ID NO:14 Peptide
2. Adjust the flow speed to “Low” and begin the run.
3. Set the forward and side scatter, as well as PMT voltages using the first Nuclease-Free Water tube according to standard Beckman Coulter FC500 procedures.
4. Once the voltages have been correctly set, increase the flow speed to “High”. Acquire 25,000 total events per replicate of each condition of each sample.
   a. PMT Voltage: Once the voltages have been set for the first Nuclease-Free Water condition, these voltages should not be changed for any of the following tubes of the same sample. The voltages may be changed between different samples but should remain the same for all runs of the same sample.
5. After sample acquisition is finished, dispose of all tubes according local disposal regulations.

### Data Analysis

Sample Analysis
1. Gate single, live cells by plotting forward vs. side scatter. Outliers containing high forward and side scatter values are assumed to be doublets and dying cells, respectively, and are excluded from the final analysis. Events that are very low in both forward and side scatter may also be excluded as cellular debris.
2. Label this gate “Live” and apply this gate to samples.
3. Select events included inside the “Live” gate.
4. Generate a dot plot of FL5 against side scatter and identify the CD45 dim population. Label this gate “CD45 dim.”
5. Using only the events within the “CD45 dim” gate, generate a dot plot of FL4 against FL3. In order to gate exclusively on AML blasts, gate cells that are high in FL4 and low in FL3. Label this gate “Blasts.”
6. Using only events within the “Blasts” population, generate a histogram of FL1. Determine the cells high in FL1 and label this gate “Polarized Cells.”
7. Apply the gates created to all aliquots of all treatments (W, D, and T) of the same sample.
8. Once the gates have been applied to all aliquots of a sample, conduct a visual inspection of each gate on each treatment to ensure that a slight difference in staining between each treatment has not due to a gate being misplaced.

Controls
1. Analyze the controls using only the “Live” gate. No other gating strategy is required for the controls because they are a pure population of one cell line.
2. Using only events within the “Live” population, generate a histogram of FL1. Determine the cells high in FL1 and label this gate “Polarized Cells.”
3. Once the “Polarized Cells” gate has been determined, analyze the control cell lines using the method described in section 9.3.

Percent Priming Calculation
1. The percent priming for each sample is calculated using the percentage of cells with the “Polarized Cells” gate.
2. Calculate the percent priming for each Peptide replicate individually using Equation 3;

“Average % Polarized Water” is the average percent of cells present in the “Polarized Cells” gate of the three water replicates, and “% Polarized Peptide” is the percent of cells in the “Polarized Cells” gate of one of the Peptide replicates.

$$\begin{matrix}Equation 3 \text{:} Equation for Calculation Percent Priming \\ \% Priming = ( \frac{\begin{matrix}( Average \% Polarized Water - \\ \% Polarized Peptide )\end{matrix}}{Average \% Polarized Water} ) \times 100\end{matrix}$$

3. The percent priming value should range from 0% to 100% for each of the replicates. Any value less than 0% will be adjusted to 0% and any value greater 100% will be adjusted to 100%.
4. The priming values for each of the replicates is then averaged and this value is multiplied by 1.6 (Equation 4).

Calibrated % Priming=Average % Priming×1.6
Equation 4: Equation for Calibration of the Average Percent Priming Value
5. The calibrated percent priming value should be below 100%. Any value greater 100% will be reported as 100%.

### Example 7

### Clinical Trial Results

A clinical trial was conducted according to the clinical trial design described in Example 5. The key eligibility criteria were: ages 18-65 years, previously untreated AML, ECOG PS 0-2, and no major organ dysfunction. Treatment consisted of increasing dose levels of alvocidib starting at 20 mg/m² as a 30-minute IV bolus followed by 30 mg/m² over 4 hours on days 1-3, cytarabine 100 mg/m²/day by continuous IV infusion on days 5-11, followed by (e.g., followed about 30 minutes later by) daunorubicin 60 mg/m² IV on days 5-7. Dose escalation portion was assessed by standard 3+3 design. All patients received a day 14 bone marrow biopsy. Reinduction therapy with alvocidib (same dose as induction) days 1-3, followed by cytarabine 100 mg/m²/day continuous IV on days 5-9, and daunorubicin 45 mg/m² IV days 5-6 was recommended in patients with >10% and >5% cellularity and blasts, respectively.

A total of 16 patients have been enrolled. The maximum dose of alvocidib administered was 30 mg/m² as a 30-minute IV bolus followed by 60 mg/m² over 4 hours on days 1-3. Overall, alvocidib was well tolerated. Cytogenetics were reported on thirteen patients; ten were intermediate- or high-risk. Screening and demographic information for the first thirteen patients enrolled in the intent-to-treat population is provided in Table 6.

TABLE 6
Screening and Demographic Information Intent-to-Treat Population.

| | | | | |
|---|---|---|---|---|
| Alvocidib | Alvocidib | Alvocidib | Alvocidib | Study |
| 20/30 mg/m² | 30/40 mg/m² | 30/50 mg/m² | 30/60 mg/m² | Total |
| (N = 3) | (N = 3) | (N = 3) | (N = 4) | (N = 13) |

Age (years)

| | | | | | | | | | | |
|---|---|---|---|---|---|---|---|---|---|---|
| n (%) | 3 | (100%) | 3 | (100%) | 3 | (100%) | 4 | (100%) | 13 | (100%) |
| Mean (S.D.) | 51.6 | (17.3) | 56.6 | (9.4) | 61.8 | (3.3) | 48.7 | (9.3) | 54.2 | (10.7) |

| | | | | | |
|---|---|---|---|---|---|
| Median | 59.2 | 61.5 | 60.8 | 45.2 | 59.2 |
| Min, Max | 31.8, 63.7 | 45.7, 62.6 | 59.2, 65.5 | 42.0, 62.4 | 31.8, 65.5 |

Sex

| | | | | | | | | | | |
|---|---|---|---|---|---|---|---|---|---|---|
| Female | 2 | (66.7%) | 2 | (66.7%) | 2 | (66.7%) | 1 | (25.0%) | 7 | (53.8%) |
| Male | 1 | (33.3%) | 1 | (33.3%) | 1 | (33.3%) | 3 | (75.0%) | 6 | (46.2%) |

Race

| American Indian or | 0 | | 0 | | 1 | (33.3%) | 0 | | 1 | (7.7%) |
|---|---|---|---|---|---|---|---|---|---|---|
| Alaska Native | | | | | | | | | | |
| Black or African | 1 | (33.3%) | 1 | (33.3%) | 0 | | 1 | (25.0%) | 3 | (23.1%) |
| American | | | | | | | | | | |
| White | 1 | (33.3%) | 2 | (66.7%) | 2 | (66.7%) | 2 | (50.0%) | 7 | (53.8%) |

| | | | | | | | | |
|---|---|---|---|---|---|---|---|---|
| Other | 1 | (33.3%) | 0 | 0 | 1 | (25.0%) | 2 | (15.4%) |

ECOG Performance Status

| | | | | | | | | | | |
|---|---|---|---|---|---|---|---|---|---|---|
| 0 | 1 | (33.3%) | 2 | (66.7%) | 2 | (66.7%) | 3 | (75.0%) | 8 | (61.5%) |

| | | | | | | | | | |
|---|---|---|---|---|---|---|---|---|---|
| 1 | 2 | (66.7%) | 0 | 1 | (33.3%) | 1 | (25.0%) | 4 | (30.8%) |

| | | | | | | | |
|---|---|---|---|---|---|---|---|
| 2 | 0 | 1 | (33.3%) | 0 | 0 | 1 | (7.7%) |

Baseline BM Blasts

| | | | | | | | | | | |
|---|---|---|---|---|---|---|---|---|---|---|
| n (%) | 3 | (100%) | 3 | (100%) | 3 | (100%) | 4 | (100%) | 13 | (100%) |
| Mean (S.D.) | 41 | (17) | 73 | (22) | 43 | (26) | 57 | (29) | 54 | (25) |

| | | | | | |
|---|---|---|---|---|---|
| Median | 35 | 79 | 34 | 53 | 49 |
| Min, Max | 28, 60 | 49, 92 | 23, 73 | 27, 93 | 23, 93 |

Fifteen patients from the study were evaluable with post baseline response assessments, and one patient was too early to assess. Eleven of the fifteen evaluable patients achieved CR, as that term is defined herein ( 12/15, 80%). One patient (No. 201) was inaspirable and, therefore, was not assessed for MRD status. Ten of the 14 evaluable patients achieved MRD negative status ( 10/14, 71%). Table 7 reports the genetic risk (e.g., according to NCCN guidelines), risk category, best response and MRD status results from the evaluable patients in the clinical trial as a function of patient and alvocidib dose level. Table 8 provides an overall summary of the results in Table 7.

TABLE 7
Genetic Risk, Risk Category, Best Response and MRD Status
of the Evaluable Patients in the Clinical Trial.

| Patient | Genetic | Risk | Dose | Best | MRD |
|---|---|---|---|---|---|
| No. | Risk | Category | Level* | Response | Status |
| 101 | Favorable | Low | 20, 30 | CR | Negative |
| 102 | Unknown | Unknown | 20, 30 | CR | Negative |
| 103 | Favorable | Low | 20, 30 | CR | Negative |
| 104 | Intermediate | Intermediate | 30, 40 | CR | Negative |
| 201 | Adverse | High | 30, 40 | CR | N/A |
| 202 | Intermediate | Intermediate | 30, 40 | CR | Negative |
| 203 | Unknown | Unknown | 30, 50 | CR | Negative |
| 204 | Adverse | High | 30, 50 | PD\hat{ } | Positive |
| 205 | Favorable | Low | 30, 50 | CR | Negative |
| 105 | Adverse | High | 30, 60 | PD\hat{ } | Positive |
| 206 | Adverse | High | 30, 60 | PD\hat{ } | Positive |
| 207 | Adverse | High | 30, 60 | CR | Negative |
| 208 | Adverse | High | 30, 60 | CR | Positive |
| 209 | Adverse | High | 30, 60 | CRᵢ | Negative |
| 301 | Intermediate | Intermediate | 30, 60 | CR | Negative |

*Hybrid dose
\hat{ }PD = progression of disease

TABLE 8
Overall Summary of the Clinical Trial Results.

| Risk Category | All Risks | | Intermediate- and High-risk | |
|---|---|---|---|---|
| Patients Evaluable | N = 15 | | N = 10 | |
| for Response | | | | |
| CR/CRᵢ (%) | 12 | (80) | 7 | (70) |
| Patients Evaluable | N = 14 | | N = 9 | |
| for MRD Status* | | | | |
| MRD⁻ (%) | 10/15 | (67) | 5/10 | (50) |
| MRD⁻ Evaluable (%) | 10/14 | (71) | 5/9 | (56) |

*One patient could not produce a sample for MRD analysis.

Alvocidib given prior to cytarabine and daunorubicin (7+3) induction in newly diagnosed AML showed an acceptable safety profile with a MTD (30 mg/m² IV bolus followed by 60 mg/m² infusion over 4 hours) similar to previously investigated timed sequential regimens of alvocidib.

All of the U.S. patents, U.S. patent application publications, U.S. patent applications, foreign patents, foreign patent applications and non-patent publications referred to in this specification are incorporated herein by reference in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating a hematologic cancer in a subject in need thereof, the method comprising administering to the subject a treatment comprising an effective amount of each of:
alvocidib, or a pharmaceutically acceptable salt thereof, or a prodrug of the foregoing on the first, second and third days of the treatment;
daunorubicin or idarubicin, or a pharmaceutically acceptable salt of the foregoing, on the fifth, sixth and seventh days of the treatment; and
cytarabine, or a pharmaceutically acceptable salt thereof, on the fifth, sixth, seventh, eighth, ninth, tenth, and eleventh days of the treatment,
wherein the treatment results in the subject being in complete remission and measurable residual disease (MRD)-negative.

2. (canceled)

3. (canceled)

4. The method of claim 1, wherein the hematologic cancer is multiple myeloma, myelodysplastic syndrome, acute myeloid leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma.

5. The method of claim 4, wherein the hematologic cancer is AML.

6. The method of claim 5, wherein the AML is MCL-1 dependent AML.

7. The method of claim 1, wherein the hematologic cancer is previously untreated.

8. The method of claim 1, wherein the subject is from 18 to 65 years old.

9. The method of claim 1, wherein the subject has an Eastern Cooperative Oncology Group performance status of 0-2.

10. The method of claim 1, comprising administering an effective amount of each of:
alvocidib, or a pharmaceutically acceptable salt thereof;
daunorubicin or idarubicin, or a pharmaceutically acceptable salt of the foregoing; and
cytarabine, or a pharmaceutically acceptable salt thereof.

11. The method of claim 1, comprising administering an effective amount of each of:
alvocidib, or a pharmaceutically acceptable salt thereof;
daunorubicin, or a pharmaceutically acceptable salt thereof; and
cytarabine, or a pharmaceutically acceptable salt thereof.

12. (canceled)

13. The method of claim 1, comprising administering to the subject from about 45 mg/m² to about 110 mg/m² daunorubicin, or a pharmaceutically acceptable salt thereof, per day.

14. The method of claim 1, wherein daunorubicin, or a pharmaceutically acceptable salt thereof, is administered to the subject intravenously.

15. The method of claim 14, wherein daunorubicin, or a pharmaceutically acceptable salt thereof, is administered to the subject via an intravenous bolus.

16. The method of claim 1, comprising administering to the subject from about 90 mg/m² to about 110 mg/m² cytarabine, or a pharmaceutically acceptable salt thereof, per day.

17. The method of claim 1, wherein cytarabine, or a pharmaceutically acceptable salt thereof, is administered to the subject intravenously.

18. The method of claim 17, wherein cytarabine, or a pharmaceutically acceptable salt thereof, is administered to the subject via intravenous infusion.

19. The method of claim 1, comprising administering to the subject from about 10 mg/m² to about 100 mg/m² alvocidib, or a pharmaceutically acceptable salt thereof, per day.

20. The method of claim 1, wherein alvocidib, or a pharmaceutically acceptable salt thereof, is administered to the subject intravenously.

21. The method of claim 20, wherein from about 25 mg/m² to about 60 mg/m² alvocidib, or a pharmaceutically acceptable salt thereof, is administered to the subject by an intravenous bolus of from about 10 minutes to about 60 minutes in duration.

22. The method of claim 20, wherein from about 25 mg/m² to about 35 mg/m² alvocidib, or a pharmaceutically acceptable salt thereof, is administered to the subject by an intravenous bolus of from about 15 minutes to about 45 minutes in duration, and about 10 mg/m² to about 65 mg/m² alvocidib, or a pharmaceutically acceptable salt thereof, is administered to the subject about 30 minutes after completion of the intravenous bolus by intravenous infusion of about 4 hours in duration.

23. The method of claim 1, further comprising detecting the MRD status of the subject.

24. The method of claim 23, further comprising terminating the treatment when detection of the subject's MRD status reveals that the subject is MRD-negative.

25. (canceled)

26. A method for treating previously untreated acute myeloid leukemia (AML) in a subject in need thereof, the method comprising administering to the subject a treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment,
wherein the treatment results in the subject being measurable residual disease (MRD)-negative.

27. The method of claim 26, comprising administering to the subject a treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
about 60 mg/m² daunorubicin, or a pharmaceutically acceptable salt thereof, per day, administered by intravenous bolus of from about 5 minutes to about 30 minutes in duration on the fifth, sixth and seventh days of the treatment; and
about 100 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the treatment.

28. (canceled)

29. The method of claim 26, comprising administering to the subject a first treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the first treatment, and the intravenous infusion is initiated about 30 minutes after completion of the intravenous bolus;
from about 45 mg/m² to about 110 mg/m² daunorubicin, or a pharmaceutically acceptable salt thereof, per day, administered by intravenous bolus of from about 5 minutes to about 30 minutes in duration on the fifth, sixth and seventh days of the first treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the first treatment; and
a second treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the second treatment, and the intravenous infusion is initiated about 30 minutes after completion of the intravenous bolus;
from about 30 mg/m² to about 60 mg/m² daunorubicin, or a pharmaceutically acceptable salt thereof, per day, administered by intravenous bolus of from about 5 minutes to about 30 minutes in duration on the fifth and sixth days of the second treatment; and
from about 90 mg/m² to about 110 mg/m² cytarabine, or a pharmaceutically acceptable salt thereof, per day, administered by intravenous infusion of from about 20 hours to about 28 hours in duration on the fifth, sixth, seventh, eighth and ninth days of the second treatment,
wherein the first day of the second treatment corresponds to the fifteenth day of the first treatment.

30. The method of claim 29, comprising administering to the subject a first treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the first treatment, and the intravenous infusion is initiated about 30 minutes after completion of the intravenous bolus;
about 60 mg/m² daunorubicin, or a pharmaceutically acceptable salt thereof, per day, administered by intravenous bolus of from about 5 minutes to about 30 minutes in duration on the fifth, sixth and seventh days of the first treatment; and
about 100 mg/m² cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the first treatment; and
a second treatment comprising:
from about 5 mg/m² to about 50 mg/m² alvocidib, or a pharmaceutically acceptable salt thereof, per day, administered by an intravenous bolus of from about 10 minutes to about 60 minutes in duration, and from about 10 mg/m² to about 65 mg/m² alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the second treatment, and the intravenous infusion is initiated about 30 minutes after completion of the intravenous bolus;
about 45 mg/m² daunorubicin, or a pharmaceutically acceptable salt thereof, per day, administered by intravenous bolus of from about 5 minutes to about 30 minutes in duration on the fifth and sixth days of the second treatment; and
about 100 mg/m² cytarabine, or a pharmaceutically acceptable salt thereof, per day, administered by intravenous infusion of from about 20 hours to about 28 hours in duration on the fifth, sixth, seventh, eighth and ninth days of the second treatment,
wherein the first day of the second treatment corresponds to the fifteenth day of the first treatment.

31. The method of claim 1, comprising administering to the subject about 90 mg/m² alvocidib, or a pharmaceutically acceptable salt thereof, per day, on the first, second and third days of the treatment.

32. The method of claim 1, comprising administering about 30 mg/m² alvocidib, or a pharmaceutically acceptable salt thereof, administered by an intravenous bolus of from about 10 minutes to about 60 minutes in duration, and about 60 mg/m² alvocidib, or a pharmaceutically acceptable salt thereof, administered by intravenous infusion of about 4 hours in duration, on each of the first, second and third days of the treatment.

33. The method of claim 26, comprising administering to the subject about 90 mg/m² alvocidib, or a pharmaceutically acceptable salt thereof, per day, on the first, second and third days of the treatment.

34. The method of claim 26, comprising administering about 30 mg/m² alvocidib, or a pharmaceutically acceptable salt thereof, per day, administered by an intravenous bolus of from about 10 minutes to about 60 minutes in duration, and about 60 mg/m² alvocidib, or a pharmaceutically acceptable salt thereof, per day, administered by intravenous infusion of about 4 hours in duration, to the subject on the first, second and third days of the treatment.

35. The method of claim 1, wherein the prodrug of alvocidib is represented by the following structural formula: 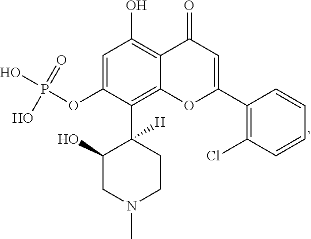 [SMILES: CN1CC[C@H](c2c(OP(=O)(O)O)cc(O)c3c(=O)cc(-c4ccccc4Cl)oc23)[C@H](O)C1]
or a pharmaceutically acceptable salt thereof.
